(19) United States
(12) Patent Application Publication
(10) Pub. No.: US 2009/0068207 A1
(43) Pub. Date: Mar. 12, 2009

(54) Compositions Containing Beta 2-Glycoprotein I-Derived Peptides for the Prevention and/or Treatment of Vascular Disease

(75) Inventors: Eyal Breitbart, Hashmonaim (IL); Niva Yacov, Tel-Aviv (IL)

Correspondence Address: MARTIN D. MOYNIHAN d/b/a PRTSI, INC., P.O. BOX 16446, ARLINGTON, VA, 22215, US

(73) Assignee: Vascular Biogenics Ltd., Or Yehuda (IL)

(21) Appl. No.: 11/918,141

(22) PCT Filed: Apr. 11, 2006
(86) PCT No.: PCT/IL2006/000467

§ 371 Date: Oct. 10, 2007

Related U.S. Application Data
(60) Provisional application No. 60/671,500, filed on Apr. 15, 2005.

(51) Int. Cl. A61K 38/00; A61K 39/00; A61P 9/00

(52) U.S. Cl. 424/184.1; 514/8

(57) ABSTRACT

Methods and compositions employing beta₂-glycoprotein-1 (β₂GPI)-derived peptides and combinations thereof effective in inducing mucosal tolerance to atheroma related antigens and effective in inhibiting inflammatory processes contributing to atheromatous vascular disease and sequalae are provided.

### FIELD OF INVENTION

The present invention relates to an immune-tolerance-inducing composition containing beta-2 glycoprotein I for the prevention and/or treatment of atherosclerosis, and uses thereof.

### BACKGROUND OF THE INVENTION

The present invention relates to β2-Glycoprotein I and associated molecules for prevention and treatment of atherosclerosis and related disease and, more particularly, to methods and compositions employing β2-Glycoprotein I and associated molecules effective in inducing immune tolerance and inhibiting inflammatory processes contributing to atheromatous vascular disease and sequalae.

Atherosclerosis

Cardiovascular disease is a major health risk throughout the industrialized world. Atherosclerosis, the most prevalent of cardiovascular diseases, is the principal cause of heart attack, stroke, and gangrene of the extremities, and as such, the principal cause of death in the United States. Atherosclerosis is a complex disease involving many cell types and molecular factors (for detailed reviews, see Ross, 1993, Nature 362: 801-809, Ross, Atherosclerosis 1997, 131Suppl.:S3-7; Schachter, Int J Card 1997; 62, Suppl. 2:S3-7; Libby, Nature 2002; 420:868-74; Zhou et al Exp Opin Biol Ther 2004; 4:599-612; Greaves at el, Trends in Immunol. 2002; 23:535-41, Martinez-Gonzales et al, Rev Esp Cardiol, 2001; 54:218-31, and Faxon et al, Circulation 2004; 109:2617-25). Currently, it is thought that atherosclerosis is the result of a response of the vascular tissues to insult or injury, endothelial dysfunction, and/or inflammation, acting to induce a cellular imbalance, causing normally anticoagulant endothelium with anticoagulant properties becomes prothrombotic (Altman, Thrombosis J. 2003; 1:4). The process, which occurs in response to insults to the endothelium and smooth muscle cells (SMCs) of the wall of the artery, consists of the formation of fatty streaks as well as fibrofatty and fibrous lesions or plaques, preceded by and associated with inflammation. The advanced lesions of atherosclerosis may occlude the artery concerned, and result from an excessive inflammatory-fibroproliferative response to numerous different forms of insult. For example, shear stresses are thought to predispose to the frequent occurrence of atherosclerotic plaques in regions of the circulatory system where turbulent blood flow occurs, such as branch points and irregular structures.

The first observable event in the formation of an atherosclerotic plaque occurs when inflammatory cells such as monocyte-derived macrophages adhere to the vascular endothelial layer and transmigrate through to the sub-endothelial space. Elevated plasma LDL levels lead to lipid engorgement of the vessel walls, with adjacent endothelial cells producing oxidized low density lipoprotein (LDL). In addition, lipoprotein entrapment by the extracellular matrix leads to progressive oxidation of LDL by lipoxygenases, reactive oxygen species, peroxynitrite and/or myeloperoxidase as well as other oxidizing compounds. These oxidized forms of LDLs are then taken up in large amounts by vascular cells through scavenger receptors expressed on their surfaces.

Lipid-filled monocytes and smooth-muscle derived cells are called foam cells, and are the major constituent of the fatty streak. Interactions between foam cells and the endothelial and smooth muscle cells surrounding them produce a state of chronic local inflammation which can eventually lead to activation of endothelial cells, increased macrophage apoptosis, smooth muscle cell proliferation and migration, and the formation of a fibrous plaque (Hajjar, D P and Haberland, M E, J. Biol Chem 1997 Sep. 12; 272(37):22975-78). Such plaques occlude the blood vessels concerned and thus restrict the blood flow, resulting in ischemia, a condition characterized by a lack of oxygen supply in tissues of organs due to inadequate perfusion. When the involved arteries block the blood flow to the heart, a person is afflicted with an acute coronary syndrome [acute myocardial infarction (MI) or unstable angina]; when the brain arteries occlude, the person experiences a stroke. When arteries to the limbs narrow, the result is severe pain, decreased physical mobility, eventually gangrene and possibly the need for amputation.

Involvement of the Immune Network in Atherosclerosis

The recognition that immune mediated processes prevail within atherosclerotic lesions stemmed from the consistent observation of lymphocytes and macrophages in the earliest stages, namely the fatty streaks. These lymphocytes, which include a predominant population of CD4+ cells (the remainder being CD8+ cells), were found to be more abundant over macrophages in early lesions, as compared with the more advanced lesions, in which this ratio tends to reverse. These findings posed questions as to whether the lymphocytes reflect a primary immune sensitization to a possible antigen or alternatively stand as a mere epiphenomenon of a previously induced local tissue damage. Regardless of the factors responsible for the recruitment of these inflammatory cells to the early plaque they seem to exhibit an activated state manifested by concomitant expression of MHC class II HLA-DR and interleukin (IL) receptor as well as leukocyte common antigen (CD45R0) and the very late antigen 1 (VLA-1) integrin.

The on-going inflammatory reaction in the early stages of the atherosclerotic lesion may either be the primary initiating event leading to the production of various cytokines by the local cells (i.e endothelial cells, macrophages, smooth muscle cells and inflammatory cells), or it may be that this reaction is a form of the body's defense immune system towards the hazardous process.

As result of chronic inflammation in atherosclerosis, numerous markers such as CRP(C-reactive protein), cytokines (interleukin-6 and 18, tumor necrosis factor α), adhesion molecules (ICAM-1), E-selectin and acute-phase reactants related to the clotting system (e.g. fibrinogen) are increased in plasma, possible predictors of further cardiovascular events. Interleukin-18 plays a key role in the inflammation cascade and is an important regulator of both innate and acquired immunities. It induces the production of interferon-γ and T-lymphocytes, has been found in human atherosclerotic lesions, and was identified as a strong independent predictor of death from cardiovascular causes in patients with stable as well as unstable angina. Inhibition of interleukin-18 reduced lesion progression with a decrease of inflammatory cells.

Matrix metalloproteinase (MMP-9) (gelatinase B), secreted by macrophages and other inflammatory cells, has been identified in various pathological processes such as general inflammation, tumor metastasis, respiratory diseases, myocardial injury, vascular aneurysms, and remodeling. MMP-9 is elevated in patients with unstable angina. A strong association has been noted between baseline MMP-9 levels and future risk of CV death, independent of IL-18. Combined determination of plasma MMP-9 and IL-18 identifies patients at very high risk.

Proinflammatory cytokines derived from monocytes, macrophages and/or adipose tissue trigger CRP in the liver. C-Reactive protein is an acute-phase reactant, a marker of inflammation, and predicts early and late mortality in patients with acute coronary syndromes. It is an independent predictor of future cardiovascular events. CRP itself promotes inflammation and atherogenesis via effects on monocytes and endothelial cells and increasing the concentration and activity of plasminogen activator inhibitor-1. CRP in atheroma participates in the pathogenesis of unstable angina and restenosis after coronary intervention. Thus, there is a vicious circle: inflammation releases proinflammatory cytokines, which in turn maintain inflammation (Altman Thrombosis J. 2003; 1:4).

The cytokines which have been shown to be upregulated by the resident cells include TNF-α, IL-1, IL-2, IL-6, IL-8, IFN-γ, IL-18 and monocyte chemoattractant peptide-1 (MCP-1). Platelet derived growth factor (PDGF) which is expressed by all cellular constituents within atherosclerotic plaques have also been shown to be overexpressed, thus possibly intensifying the preexisting inflammatory reaction by a co-stimulatory support in the form of a mitogenic and chemotactic factor. Recently, Uyemura K et al (J Clin Invest 1996 97; 2130-2138) have elucidated type 1 T-cell cytokine pattern in human atherosclerotic lesions exemplified by a strong expression of IFN-γ but not IL-4 mRNA in comparison with normal arteries. Furthermore, IL-12-a T-cell growth factor produced primarily by activated monocytes and a selective inducer of Th1 cytokine pattern, was found to be overexpressed within lesions as manifested by the abundance of its major heterodimer form p70 and p40 (its dominant inducible protein) mRNA.

Similar to the strong evidence for the dominance of the cellular immune system within the atherosclerotic plaque, there is also ample data supporting the involvement of the local humoral immune system. Deposition of immunoglobulins and complement components have been shown in the plaques in addition to the enhanced expression of the C3b and C3Bi receptors in resident macrophages. In a recent study, Caligiuri et al disclosed that B cells from apoE ° mice inhibit atherosclerosis in splenectomized and intact mice (Caligiuri et al, J Clin Invest, 2002, 109:745-53). Similarly, studies involving immunization of animals with plaque related antigens indicate the contribution of humoral immunity to attenuation of plaque formation and inhibition of atherosclerosis (see, for example, George et al, Atherosclerosis, 1998, 138; 147-152; Zhou et al, Arterioscler Thromb Vasc Biol 2001; 21:108-14; and Freigang, et al. 1998; 1972-82).

Atherosclerosis and Inflammation

Valuable clues with regard to the contribution of immune mediated inflammation to the progression of atherosclerosis comes from animal models. Hence, it seems that immunocompromised mice (class I MHC deficient) tend to develop accelerated atherosclerosis as compared with immune competent mice. Additionally, treatment of C57BL/6 mice (Emeson E E, Shen M L. Accelerated atherosclerosis in hyperlipidemic C57BL/6 mice treated with cyclosporine A. Am J Pathol 1993; 142: 1906-1915) and New-Zealand White rabbits (Roselaar S E, Schonfeld G, Daugherty A. Enhanced development of atherosclerosis in cholesterol fed rabbits by suppression of cell-mediated immunity. J Clin Invest 1995; 96: 1389-1394) with cyclosporine A, which is a potent suppressor of IL-2 transcription resulted in a significantly enhanced atherosclerosis under “normal” lipoprotein “burden”. More recently, it has been demonstrated that cyclosporin A-related autoreactive mechanisms contribute to the high incidence of graft vasculopathy (Chen, Cli Immunol 2001; 100:57-70). These latter studies may provide insight into the possible roles of the immune system as engaged in counteracting the self-perpetuating inflammatory process within the atherosclerotic plaque.

Oxidized LDL has been implicated in the pathogenesis of atherosclerosis and atherothrombosis, by it's action on monocytes and smooth muscle cells, and by inducing endothelial cell apoptosis, impairing anticoagulant balance in the endothelium. Oxidized LDL also inhibits anti-atherogenic HDL-associated breakdown of oxidized phospholipids (Mertens, A and Holvoet, P, FASEB J 2001 October; 15(12):2073-84). This association is also supported by many studies demonstrating the presence of oxidized LDL in the plaques in various animal models of atherogenesis; the retardation of atherogenesis through inhibition of oxidation by pharmacological and/or genetic manipulations; and the promising results of some of the interventional trials with anti-oxidant vitamins (see, for example, Witztum J and Steinberg, D, Trends Cardiovasc Med 2001 April-May; 11(3-4):93-102 for a review of current literature). Indeed, oxidized LDL and malondialdehyde (MDA)-modified LDL have been recently proposed as accurate blood markers for 1^{st} and 2^{nd} stages of coronary artery disease (U.S. Pat. Nos. 6,309,888 to Holvoet et al and 6,255,070 to Witztum, et al).

Reduction of LDL oxidation and activity has been the target of a number of suggested clinical applications for treatment and prevention of cardiovascular disease. Bucala, et al (U.S. Pat. No. 5,869,534) discloses methods for the modulation of lipid peroxidation by reducing advanced glycosylation end product, lipid characteristic of age-, disease- and diabetes-related foam cell formation. Tang et al, at Incyte Pharmaceuticals, Inc. (U.S. Pat. No. 5,945,308) have disclosed the identification and proposed clinical application of a Human Oxidized LDL Receptor in the treatment of cardiovascular and autoimmune diseases and cancer.

Beta₂-Glycoprotein I

Another abundant atherogenesis-related plaque component is Beta₂-Glycoprotein I. Beta₂-Glycoprotein I (β₂GPI) is a 50-kDa molecule that acts as an anticoagulant in in-vitro assays. Although the exact role of β₂GPI in atherogenesis has yet to be elucidated, several relevant properties have been observed: 1) it is able to bind immobilized negatively charged phospholipids or phospholipid-expressing cells (apoptotic cells, activated platelets); 2) it is able to bind to modified cellular surfaces, enhancing their clearance by scavenging macrophages (Chonn A, et al J Biol Chem 1995; 270: 25845-49; and Thiagarajan P, et al Arterioscler Thromb Vasc Biol 1999; 19:2807-11); and 3) it is an important target for binding of autoimmune antiphospholipid antibodies (aPLs). β₂GPI has to undergo structural alteration in order to be recognized by aPLs. This alteration may be initiated, for example, by binding to negatively charged phospholipids or high-binding plates, but also in-vivo by binding apoptotic cells that express phosphatidylserine.

Recent studies investigating the importance of anti β₂GPI antibodies in promoting a procoagulant state have focused on the effects of these antibodies on cellular and protein components of the coagulation system (endothelial cells, platelets and macrophages; tissue factor and coagulation factors). These studies indicate that anti β₂GPI antibodies prevent the deactivation of platelets, sustaining their phagocytic clearance; interact with late endosomes of human endothelial cells; and suppress the inhibitory activity of the tissue factor pathway inhibitor. This association with coagulation events is consistent with β₂GPIs proposed function in the prothrombotic antiphospholipid syndrome (APLS). U.S. Pat. Nos. 5,998,223 and 5,344,758 (to Matsuura, et al and Krilis, et al, respectively), and US Patent Application No. 20030100036 and Ser. No. 10/488,688 to Vojdani et al. and Matsuura et al., respectively, disclose the application of anti β₂GPI antibodies, some to cryptic epitopes, for diagnostics in APLS and SLE. U.S. Pat. No. 5,900,359 to Matsuura et al teaches the use of anti-β₂GPI for the detection of circulating oxidized LDL via Ox-LDL-β₂GPI complexes. U.S. patent application Ser. No. 10/694,033 to Berg et al. discloses the detection of anti-β2GPI antibodies for early diagnosis of activation of coagulation response in vascular and clotting disorders. Koike et al (U.S. patent application Ser. No. 10/429,479) teaches the determination of nicked β₂GPI in blood samples for diagnosing cerebral infarct. However, no therapeutic applications are disclosed by the authors.

The antigenic properties of β₂GPI-cardiolipin complex, and their association with anti-PL antibody related diseases has led some researchers to propose the use of β₂GPI or β₂GPI sequences as B-cell toleragens in the treatment of anti-PL antibody related disease such as recurrent stroke and recurrent fetal loss (see U.S. patent application Ser. No. 10/044,844 and U.S. Pat. No. 5,874,409 to Victoria et al.).

Since aminophospholipids have been identified as readily accessible markers in the walls of tumor blood vessels, the application of anti-β₂GPI antibodies for therapy in cancer and tumorigenesis has been proposed (see, for example, Thorpe et al U.S. patent application Ser. Nos. 09/990,833 and 10/259,244, and U.S. Pat. Nos. 6,818,213 and 6,783,760). The use of β₂GPI in an anti-cancer vaccine is taught by Schroit in U.S. Pat. No. 6,806,354 and U.S. patent application Ser. No. 09/974,753.

Immunization of atherosclerosis-prone LDL R−/− mice by subcutaneous injection of human β₂GPI emulsified in complete Freunds adjuvant resulted in high titers of anti-β₂GPI antibodies, detectable amounts of circulating immune complexes with β₂GPI, and induction of increased plaque formation and other indicators of early atherogenesis (George et al, Circulation 1998; 98: 1108-1115).

Heat Shock Protein (HSP)

A third important plaque-related component associated with atherogenesis is the 60/65 kDa heat shock protein (HSP). This mitochondrial protein is a member of the HSP family, which constitutes nearly 24 proteins displaying high degree of sequence homologies between different species. These proteins, as their name implies, are expressed in response to stresses including exposure to free radicals, heat, mechanical shear stress, infections and cytokines, and protect against unfolding and denaturation of cellular proteins. This has led to their designation as molecular ‘chaperones’. However, HSP function may have undesired consequences, since over expression of HSPs may, under certain conditions promote an autoimmune reaction with resultant tissue damage. The mechanisms responsible for the HSP immune mediated damage are as yet unclear: it is presumed that cryptic, “non-self” neo-epitopes are exposed following their upregulation. Alternatively, it was suggested that cross-reaction exists between self-HSP and ‘foreign’ HSP epitopes introduced following infections which may trigger a pathological, autoimmune response against native HSP. Support for the involvement of HSP in autoimmunity is provided by studies documenting enhanced autoantibody as well as cellular response to HSP 60/65 in several autoimmune diseases (Schoenfeld, Y et al Autoimmunity 2000 September; 15(2):199-202; U.S. Pat. No. 6,130,059 to Covacci, et al; and Gromadza G, et al Cerebrovascul Dis 2001, October; 12(3):235-39).

The link between HSP 65 and atherosclerosis was initially recognized by George Wick's group, who found that normocholesterolemic rabbits immunized with different antigens developed atherosclerosis, provided the preparation used for immunization contained complete Freund's adjuvant (CFA)(Xu, Q, et al Arterioscler Thromb 1992; 12:789-99). Since the major constituent of CFA is heat killed mycobacterium tuberculosis, the principal component of which is the HSP-65, they reasoned that the immune response towards this component led to the development of atherosclerosis. This was confirmed when these authors demonstrated that immunization of animals with HSP 65 produced pronounced atherosclerosis, and that T cells from experimentally atherosclerotic rabbits overexpressed HSP-65, indicating a localized immune reaction restricted to the stressed arterial vessel. The importance of endogenous HSP-65 in atherogenesis was further demonstrated by the acceleration of fatty streak formation following HSP-65 (or Mycobacterium tuberculosis) immunization of naïve mice (George J, et al Arterioscler Thromb Vasc Biol 1999; 19:505-10;).

Involvement of humoral immune mechanisms in response to HSP-65 were observed in atherosclerosis: a marked correlation has been found between high levels of anti-HSP65 antibodies and the extent of sonographically estimated carotid narrowing in a screen of healthy individuals (Xu Q. et al Lancet 1993; 341: 255-9; Xu Q. et al Circulation 1999; 100 (11):1169-74). In addition, in-vitro experiments with cultured endothelial cells have demonstrated the concentration and time dependent induction of endothelial cell adhesion to monocytes and granulocytes following incubation with HSP65.

Atherosclerosis and Autoimmune Disease

Because of the presumed role of the excessive inflammatory-fibroproliferative response in atherosclerosis and ischemia, a growing number of researchers have attempted to define an autoimmune component of vascular injury. In autoimmune diseases the immune system recognizes and attacks normally non-antigenic body components (autoantigens), in addition to attacking invading foreign antigens. The autoimmune diseases are classified as auto- (or self-) antibody mediated or cell mediated diseases. Typical autoantibody mediated autoimmune diseases are myasthenia gravis and idiopathic thrombocytopenic purpura (ITP), while typical cell mediated diseases are Hashimoto's thyroiditis and type I (Juvenile) Diabetes.

Atherosclerosis is not a classical autoimmune disease, although some of its manifestations such as the production of the plaque that obstructs the vasculature may be related to aberrant immune responsiveness. In classical autoimmune disease, one can often define very clearly the sensitizing autoantigen attacked by the immune system and the component(s) of the immune system which recognize the autoantigen (humoral, i.e. autoantibody or cellular, i.e. lymphocytes). Above all, one can show that by passive transfer of these components of the immune system the disease can be induced in healthy animals, or in the case of humans the disease may be transferred from a sick pregnant mother to her offspring. Many of the above are not prevailing in atherosclerosis. Atherosclerosis, and its related conditions, can by no means be considered a classic autoimmune disease.

Indeed, much of the prior art teaches away from the inclusion of atherosclerosis as a classic autoimmune disease. Autoimmune diseases or conditions are defined as those in which an immune response (humoral or cellular) possess pathogenic properties that should be either identified in an autoimmune state or be transferable to non-immune animals (Harrison's Textbook of Internal Medicine, Autoimmune Diseases).

Atherosclerosis progresses gradually and does not have the classic flare and remission of classic autoimmune disease. Indeed, unlike other autoimmune diseases, atherosclerosis does not respond to corticosteroids or immune suppressants: treatment with cyclosporin A further aggravates the disease (Emeson et al Am J Pathol 1993; 142: 1906-15). In fact, Meir et al, in a recent review of the contribution of inflammation to atherosclerosis in humans (Commentaries, Int. Atheroscler Soc.) concluded that “thus far there is neither cogent clinical evidence that anti-inflammatory agents decrease vascular morbidity or mortality, nor cogent evidence linking them to decreased atherogenesis in humans. Inflammation may simply be a marker of active disease”. In addition, the disease definitely has common risk factors such as hypertension, diabetes, lack of physical activity, smoking and others, the disease affects elderly people and has a different genetic preponderance than in classical autoimmune diseases.

Treatment of inflammatory disease may be directed towards suppression or reversal of general and/or disease-specific immune reactivity. Thus Aiello, for example (U.S. Pat. Nos. 6,034,102 and 6,114,395) discloses the use of estrogen-like compounds for treatment and prevention of atherosclerosis and atherosclerotic lesion progression by inhibition of inflammatory cell recruitment. Similarly, Medford et al (U.S. Pat. No. 5,846,959) disclose methods for the prevention of formation of oxidized PUFA, for treatment of cardiovascular and non-cardiovascular inflammatory diseases mediated by the cellular adhesion molecule VCAM-1. Furthermore, Falb (U.S. Pat. No. 6,156,500) designates a number of cell signaling and adhesion molecules abundant in atherosclerotic plaque and disease as potential targets of anti-inflammatory therapies. Colon-Cruz et al. (U.S. Pat. No. 6,821,964) teach the use of chemokines receptor modulators (CCR2 and CCR3 antagonists) for treatment of atherosclerosis. Tracey (U.S. Pat. No. 6,610,713) teaches treatment of atherosclerosis by the inhibition of inflammatory cytokines release with cholinergic agonists and vagus nerve stimulation. Benyunes et al (U.S. patent application Ser. No. 10/818,765) discloses the use of a surface marker-targeted B-cell antagonist for boosting the inhibition of TNF-Y in autoimmune disease.

Since oxidized LDL, β₂GPI and HSP 65 have been clearly implicated in the pathogenesis of atherosclerosis (see above), the contribution of these prominent plaque components to autoimmunity in atheromatous disease processes has been investigated.

Immune Responsiveness to Plaque Associated Molecules

It is known that Ox LDL is chemotactic for T-cells and monocytes. Ox LDL and its byproducts are also known to induce the expression of factors such as monocyte chemotactic factor 1, secretion of colony stimulating factor and platelet activating properties, all of which are potent growth stimulants.

The active involvement of the cellular immune response in atherosclerosis has been substantiated (see, for example, Stemme S, et al, Proc Natl Acad Sci USA 1995; 92: 3893-97), by detection of isolated CD4+ within plaques clones responding to Ox LDL as stimuli. The clones corresponding to Ox LDL (4 out of 27) produced principally interferon-γ rather than IL-4. It remains to be seen whether the above T-cell clones represent mere contact of the cellular immune system with the inciting strong immunogen (Ox LDL) or that this reaction provides means of combating the apparently indolent atherosclerotic process.

The data regarding the involvement of the humoral mechanisms and their meaning are much more controversial. One recent study reported increased levels of antibodies against MDA-LDL, a metabolite of LDL oxidation, in women suffering from heart disease and/or diabetes (Dotevall, et al., Clin Sci 2001 November; 101(5): 523-31). Other investigators have demonstrated antibodies recognizing multiple epitopes on the oxidized LDL, representing immune reactivity to the lipid and apolipoprotein components (Steinerova A, et al., Physiol Res 2001; 50(2): 131-41) in atherosclerosis and other diseases, such as diabetes, renovascular syndrome, uremia, rheumatic fever and lupus erythematosus. Several reports have associated increased levels of antibodies to Ox LDL with the progression of atherosclerosis (expressed by the degree of carotid stenosis, severity of peripheral vascular disease etc.). Most recently, Sherer et al (Cardiology 2001; 95(1):20-4) demonstrated elevated levels of antibodies to cardiolipin, β-2GPI and oxLDL, but not phosphatidylcholine or endothelial cells in coronary heart disease. Thus, there seems to be a consensus as to the presence of anti-plaque-component antibodies in the form of immune complexes within atherosclerotic plaque, but uncertainty as to their role in atherogenesis.

Regarding the immunogenicity of β₂GPI, it has been shown that β₂GPI serves as a target antigen for an immune-mediated attack, influencing the progression of atherosclerosis in humans and mice. George J et al. immunized LDL-receptor deficient mice with β₂GPI, producing a pronounced humoral immune response to human Beta2GPI, and larger early atherosclerotic lesions in comparison with controls (George J, et al Circulation 1998; 15:1108-15). Afek A, et al obtained similar results in atherosclerosis-prone apolipoprotein-E-knockout mice immunized once with human β2GPI and fed a high fat diet for 5 weeks (Afek A et al. Pathobiology 1999; 67:19-25).

Further, although immune reactivity to β₂GPI in humans with the prothrombotic antiphospholipid syndrome has traditionally been attributed to the presence of autoantibodies to β₂GPI, recent observations have indicated the importance of a cellular immune response to β₂GPI. T-cells reactive with β₂GPI have been demonstrated in the peripheral blood of patients with antiphospholipid syndrome. These T cells displayed a T-helper-1 phenotype (secreting the proinflammatory and proatherogenic cytokine interferon-γ) and were also capable of inducing tissue factor production (Visvanathan S, and McNiel H P. J Immunolog 1999; 162:6919-25). Taken together, the abundant data gathered to date regarding anti β₂GPI (for review see Roubey R A, Curr Opinion Rheumatol 2000; 12:374-378), indicates that the immune response to this plaque related antigen may play a significant role in influencing the size and composition of atherosclerotic plaque.

Finally, there exists a significant dependency between the antigenicity, and pathogenicity of oxidized phospholipids and β₂GPI. As mentioned above, some of the autoimmune epitopes associated with minimally modified LDL and β₂GPI are cryptic. Kyobashi, et al (J Lipid Res 2001; 42:697-709), and Koike, et al (Ann Med 2000; 32:Suppl I 27-31) have identified a macrophage-activating oxLDL specific ligand present only with β₂GPI-OxLDL complex formation. This ligand was recognized by APLS-specific autoantibodies. Thus, there is evidence from both laboratory and clinical studies for the pathogenic role of β₂GPI and other plaque components, and their importance as autoantigens in atherosclerosis, as well as other diseases.

Beta₂Glycoprotein I-Derived Peptides

Many studies have attempted to identify those portions of Beta₂-Glycoprotein I peptide sequence responsible for the anti-Phospholipid Syndrome and anti-Cardiolipin related actions of the whole antigen. Since the interaction with anti-PL and anti-CL antibodies is thought to be critical for the coagulation and thrombogenic effects observed in these conditions, most studies have investigated anti-PL and anti-CL binding epitopes.

Gharavi et al (Arthritis-Rheum 2002; 46:545-52; and Lupus, 2004; 13-17-23) have identified a CMV-derived peptide which can be used to produce antibodies which mimic the fetal loss and thrombosis of the aPL syndrome. Blank et al (J Clin Invest 2002; 106:797-804) identified a bacterial antigen that induced anti-β₂GPI antibodies, inferring an infectious etiology of the aPL syndrome. Iverson, et al (Immunology 1998; 95:15542-46) used anti-β₂GPI antibodies from patients with aPL syndrome to identify an epitope located in domain 1 of human β₂GPI. Jones et al (BioConjugates Chem 1999; 10:480-488; Bioconjugates Chem 2001; 12:1012-20) synthesized synthetic peptides mimicking β₂GPI sequences, which bind to anti-β₂GPI antibodies, in the hope of finding β-cell toleragens for suppressing anti-β₂GPI humoral immunity. Similarly, Meroni et al (Lupus 1998; Suppl 2: S44-47) identified a synthetic anti-β₂GPI-binding peptide having homology to a portion of the CL binding site of β₂GPI. Using an overlapping 12-mer peptide library covering the entire human β2GPI sequence, Ito et al. (Humoral Immunity 2000; 61:366-77) identified specific regions of the β₂GPI polypeptide implicated in T-cell responses to β₂GPI. However, none of the studies related to induction of tolerance by mucosal administration, or to the effects of the β₂GPI peptides on atherosclerosis or related disease.

Pierangeli et al. (J Autoimmunity 2004; 22:217-25) reported a synthetic peptide that mimics portions of bacterial proteins and β₂GPI that inhibits the thrombogenic activity of anti-PL antibodies when injected in vivo. Taking another approach, Blank et al (PNAS USA 1999; 96:5164-68) identified three synthetic hexapeptide antigens corresponding to β2GPI domains, which bind to anti-PL or anti-CL antibodies, and could be used for blocking the interaction of β₂GPI and anti-β₂GPI in the anti-PL syndrome.

Thus, potential candidate peptides for blocking formation of anti-β₂GPI-β₂GPI complex, and thus inhibiting the thrombosis and coagulation of aPL syndrome have been identified. However, no mucosal administration, or anti-atherogenic therapy was envisaged.

Mucosal Tolerance in Treatment of Autoimmune Disease

Recently, new methods and pharmaceutical formulations have been found that are useful for treating autoimmune diseases (and related T-cell mediated inflammatory disorders such as allograft rejection and retroviral-associated neurological disease). These treatments induce tolerance, orally or mucosally, e.g. by inhalation, using as tolerizers autoantigens, bystander antigens, or disease-suppressive fragments or analogs of autoantigens or bystander antigens. Such treatments are described, for example, in U.S. Pat. Nos. 5,935,577, 6,019,970, 6,790,447, 6,703,361, 6,645,504, 5,961,977, 6,077,509, to Weiner et al., 5,843,449 to Boots et al., and U.S. patent application Ser. Nos. 10/451,370, 10/989,724, 09/944,592, 09/806,400, PCT Nos. IL99/00519 and IL02/00005 and Israel Patent Application No. 126447 to Harats et al., and in George et al., “Suppression of early atherosclerosis in LDL receptor deficient mice by oral tolerance with beta2 glycoprotein I”, Cardiovascular Research 2004; 62:603-09, (which are incorporated herein by reference, as if fully set forth). Autoantigens and bystander antigens are defined below (for a general review of mucosal tolerance see Nagler-Anderson, C., Crit. Rev Immunol 2000; 20(2):103-20, and Weiner et al. Microb Infect 2001; 3:947-54). Intravenous administration of autoantigens (and fragments thereof containing immunodominant epitopic regions of their molecules) has been found to induce immune suppression through a mechanism called clonal anergy. Clonal anergy causes deactivation of only immune attack T-cells specific to a particular antigen, the result being a significant reduction in the immune response to this antigen. Thus, the autoimmune response-promoting T-cells specific to an autoantigen, once anergized, no longer proliferate in response to that antigen. This reduction in proliferation also reduces the immune reactions responsible for autoimmune disease symptoms (such as neural tissue damage that is observed in multiple sclerosis; MS). There is also evidence that oral administration of autoantigens (or immunodominant fragments) in a single dose and in substantially larger amounts than those that trigger “active suppression” may also induce tolerance through anergy (or clonal deletion).

A method of treatment has also been disclosed that proceeds by active suppression. Active suppression functions via a different mechanism from that of clonal anergy. This method, discussed extensively in PCT Application PCT/US93/01705, involves oral or mucosal administration of antigens specific to the tissue under autoimmune attack. These are called “bystander antigens”. This treatment causes regulatory (suppressor) T-cells to be induced in the gut-associated lymphoid tissue (GALT), or bronchial associated lymphoid tissue (BALT), or most generally, mucosa associated lymphoid tissue (MALT) (MALT includes GALT and BALT). These regulatory cells are released in the blood or lymphatic tissue and then migrate to the organ or tissue afflicted by the autoimmune disease and suppress autoimmune attack of the afflicted organ or tissue. The T-cells elicited by the bystander antigen (which recognize at least one antigenic determinant of the bystander antigen used to elicit them) are targeted to the locus of autoimmune attack where they mediate the local release of certain immunomodulatory factors and cytokines, such as transforming growth factor beta (TGF beta), interleukin-4 (IL-4), and/or interleukin-10 (IL-10). Of these, TGF-beta is an antigen-nonspecific immunosuppressive factor in that it suppresses immune attack regardless of the antigen that triggers the attack. (However, because oral or mucosal tolerization with a bystander antigen only causes the release of TGF-beta in the vicinity of autoimmune attack, no systemic immunosuppression ensues.) IL-4 and IL-10 are also antigen-nonspecific immunoregulatory cytokines. IL-4 in particular enhances (T helper 2) Th₂ response, i.e., acts on T-cell precursors and causes them to differentiate preferentially into Th₂ cells at the expense of Th₁ responses. IL-4 also indirectly inhibits Th₁ exacerbation. IL-10 is a direct inhibitor of Th₁ responses. After orally tolerizing mammals afflicted with autoimmune disease conditions with bystander antigens, increased levels of TGF-beta, IL-4 and IL-10 are observed at the locus of autoimmune attack (Chen, Y. et al., Science, 265:1237-1240, 1994). The bystander suppression mechanism has been confirmed by von Herreth et al. (J. Clin. Invest., 96:1324-1331, September 1996).

More recently, oral tolerance has been effectively applied in treatment of animal models of inflammatory bowel disease by feeding probiotic bacteria (Dunne, C, et al., Antonie Van Leeuwenhoek 1999 July-November; 76(1-4):279-92), autoimmune glomerulonephritis by feeding glomerular basement membrane (Reynolds, J. et al., J Am Soc Nephrol 2001 January; 12(1): 61-70) experimental allergic encephalomyelitis (EAE, which is the equivalent of multiple sclerosis or MS), by feeding myelin basic protein (MBP), adjuvant arthritis and collagen arthritis, by feeding a subject with collagen and HSP-65, respectively. A Boston based company called Autoimmune has carried out several human experiments for preventing diabetes, multiple sclerosis, rheumatoid arthritis and uveitis. The results of the clinical trials have been less impressive than the animal experiments, however there has been some success with the prevention of arthritis.

Oral tolerance to autoantigens found in atherosclerotic plaque lesions has also been investigated. Study of the epitopes recognized by T-cells and Ig titers in clinical and experimental models of atherosclerosis indicated three candidate antigens for suppression of inflammation in atheromatous lesions: oxidized LDL, the stress-related heat shock protein HSP 65 and the cardiolipin binding protein β₂GPI. U.S. patent application Ser. No. 09/806,400 to Shoenfeld et al (filed Sep. 30, 1999), and U.S. patent application Ser. Nos. 10/451,370 and 10/989,724 to Harats et al., which are incorporated by reference herein in their entirety, disclose the reduction by approximately 30% of atherogenesis in the arteries of genetically susceptible LDL receptor deficient mice (LDL-RD) fed oxidized human LDL and other atheroma related antigens. Although significant inhibition of atherogenesis was achieved, presumably via oral tolerance, no identification of specific lipid antigens or immunogenic LDL components was made. Another obstacle encountered was the inherent instability of the orally fed antigen in vivo, due to digestive breakdown, and uptake of oxidized LDL by the liver and cellular immune mechanisms. It is plausible that a mucosal route of administration other than feeding (oral) would have provided tolerance of greater efficiency.

The induction of immune tolerance and subsequent prevention or inhibition of autoimmune inflammatory processes has been demonstrated using exposure to suppressive antigens via mucosal. The membranous tissue around the eyes, the middle ear, the respiratory and other mucosa, and especially the mucosa of the nasal cavity, like the gut, are exposed to many invading as well as self-antigens and possess mechanisms for immune reactivity. Thus, Rossi, et al (Scand J Immunol 1999 August; 50(2):177-82) found that nasal administration of gliadin was as effective as intravenous administration in downregulating the immune response to the antigen in a mouse model of celiac disease. Similarly, nasal exposure to acetylcholine receptor antigen was more effective than oral exposure in delaying and reducing muscle weakness and specific lymphocyte proliferation in a mouse model of myasthenia gravis (Shi, F D. et al, J Immunol 1999 May 15; 162 (10): 5757-63), intranasal or aerosol administration of pancreatic islet autoantigen in prediabetic mice reduced the incidence of diabetes (Hanninen et al., Immunol Rev 2000; 173:109-19), intranasal administration of Staph enterotoxin A protected mice against toxic shock syndrome (Collins, et al., Infect Immun 2002; 70:2282-87, and nasal tolerance to E-selectin inhibited ischemic and hemorrhagic stroke in hypertensive stroke-prone (SHR-SP) rats (Takeda, et al., Stroke 2002; 33:2156) Therefore, immunogenic compounds intended for mucosal as well as intravenous or intraperitoneal administration should be adaptable to oral, nasal and other membranous routes of administration.

The current treatments for the prevention and treatment of atherosclerosis include certain pharmacological approaches, in addition to alteration of lifestyle factors which can ameliorate atherosclerosis, such as diet control, weight loss, increased exercise, and smoking cessation. Examples of pharmacological agents in current use for the treatment and prevention of atherosclerosis are hydroxymethylglutaryl-coenzyme A (HMGCoA) reductase inhibitors (statins) to control high LDL, nicotinic acid to control high lipoprotein (a) and low high density lipoprotein (HDL), and fibric acid derivatives to control high levels of triglycerides. Adjunctive pharmacological treatment includes measures directed toward control of diabetes mellitus and hypertension.

In view of the foregoing, a need still exists to develop methods and compositions for treating and/or preventing vascular disorders such as atherosclerosis. Preferably, such methods and compositions would include non-invasive modes of administration and, more preferably, be based, in part, on the molecular interactions which mediate an inflammatory response. Thus, there is clearly a need for novel methods of employing, and compositions containing β2 glycoprotein I-derived peptides capable of superior tolerizing immunogenicity in mucosal administration.

### SUMMARY OF THE INVENTION

According to one aspect of the present invention there is provided a method for prevention and/or treatment of a vascular condition in a subject in need thereof, the method effected by administering to a mucosal surface of the subject a mucosal tolerance-inducing amount of at least one beta₂-glycoprotein-1 (β₂GPI) derived peptide, thereby inducing mucosal tolerance.

According to further features in preferred embodiments of the invention described below the vascular condition is selected from the group consisting of atherosclerosis, cardiovascular disease, cerebrovascular disease, peripheral vascular disease, stenosis, restenosis and/or in-stent-stenosis.

According to an additional aspect of the present invention there is provided a method for modulating an immune response to a beta₂-glycoprotein-1 (β₂GPI) in a subject in need thereof, the method effected by administering to a mucosal surface of the subject a mucosal tolerance-inducing amount of at least one beta₂-glycoprotein-1 (β₂GPI) derived peptide, thereby inducing mucosal tolerance and modulating the immune response to the β₂GPI.

According to yet another aspect of the present invention there is provided a method for modulating an immune response to an atheroma plaque-related antigen in a subject in need thereof, the method effected by administering to a mucosal surface of the subject a mucosal tolerance-inducing amount of at least one beta₂-glycoprotein-1 (β₂GPI)-derived peptide, thereby inducing mucosal tolerance and modulating the immune response to the atherosclerotic plaque antigen.

According to further features in preferred embodiments of the invention described below, the at least one beta₂-glycoprotein-1 (β₂GPI)-derived peptide comprises a combination of at least two beta₂-glycoprotein-1 (β₂GPI)-derived peptides.

According to yet another aspect of the present invention there is provided a pharmaceutical composition for prevention and/or treatment of a vascular condition in a subject in need thereof comprising as an active ingredient a combination of at least two beta₂-glycoprotein-1 (β₂GPI)-derived peptides and a pharmaceutically acceptable carrier.

According to an additional aspect of the present invention there is provided a pharmaceutical composition for modulating an immune response to a beta₂-glycoprotein-1 (β₂GPI) in a subject in need thereof comprising as an active ingredient a combination of at least two beta₂-glycoprotein-1 (β₂GPI)-derived peptides and a pharmaceutically acceptable carrier.

According to yet another aspect of the present invention there is provided a pharmaceutical composition for modulating an immune response to an atheroma plaque-related antigen in a subject in need thereof comprising as an active ingredient a combination of at least two beta₂-glycoprotein-1 (β₂GPI)-derived peptides and a pharmaceutically acceptable carrier.

According to further features in preferred embodiments of the invention described below the at least one β₂GPI-derived peptide is a human β₂GPI-derived peptide.

According to yet further features in preferred embodiments of the invention as described below, the at least one β₂GPI-derived peptide is a synthetic peptide.

According to yet further features in preferred embodiments of the invention as described below, the at least one β₂GPI-derived peptide has a sequence as set forth in one of SEQ ID NOs: 25-57,315.

According to yet further features in preferred embodiments of the invention as described below, the combination of at least two β₂GPI-derived peptides is a mixture of peptides.

According to still further features in preferred embodiments of the invention as described below, the combination of at least two β₂GPI-derived peptide is a chimeric peptide comprising at least two β₂GPI-derived peptides in covalent linkage.

According to yet further features in preferred embodiments of the invention as described below, the chimeric peptide comprises a first β₂GPI-derived peptide having a sequence as set forth in one of SEQ ID NOs: 25-57,315 covalently linked to a second β₂GPI-derived peptide having a sequence as set forth in any of SEQ ID NOs: 25-57,315.

According to yet further features in preferred embodiments of the invention as described below, the pharmaceutical composition is formulated for mucosal administration.

According to still further features in preferred embodiments of the invention described below the pharmaceutical composition further includes a therapeutically effective amount of at least one additional compound selected from the group consisting of HMGCoA reductase inhibitors (statins), mucosal adjuvants, corticosteroids, anti-inflammatory compounds, analgesics, growth factors, toxins, and additional tolerizing antigens.

According to yet further features in preferred embodiments of the invention described below, the administering is effected by oral, enteral, buccal, nasal, bronchial, intrapulmonary or intra-peritoneal administration.

According to still further features in preferred embodiments of the invention described below the method further includes administering a therapeutically effective amount of at least one additional compound selected from the group consisting of HMGCoA reductase inhibitors (statins), mucosal adjuvants, corticosteroids, anti-inflammatory compounds, analgesics, growth factors, toxins, and additional tolerizing antigens.

According to further features in preferred embodiments of the invention described below, modulating is reducing immune reactivity to β₂GPI in the subject.

According to yet further features in preferred embodiments of the invention described below the immune response is selected from the group consisting of Th1 type cytokines expression, Th2 type cytokines expression, and T-cell proliferation.

According to further features in preferred embodiments of the invention described below the atheroma plaque-related antigen is selected from the group consisting of beta₂-glycoprotein-1 (β₂GPI), oxidized LDL (oxLDL) and heat shock protein (HSP 60/65).

According to still further features in preferred embodiments of the invention described below, modulating is reducing immune reactivity to the atherosclerotic plaque-related antigen in the subject.

According to an additional aspect of the present invention there is provided an article of manufacture, packaged and identified for use in modulating an immune response to an atherosclerotic plaque antigen in a subject in need thereof. The article of manufacture includes a packaging material and a mucosal tolerance-inducing amount of an active ingredient selected from the group consisting of at least one beta₂-glycoprotein-1 (β₂GPI)-derived peptide, and the packaging material includes a label or package insert indicating that the mucosal tolerance-inducing amount of the active ingredient is for modulating an immune response to an atherosclerotic plaque antigen in the subject via mucosal administration.

According to further features in preferred embodiments of the invention described below the atheroma plaque-related antigen is selected from the group consisting of beta₂-glycoprotein-1 (β₂GPI), oxidized LDL (oxLDL) and heat shock protein (HSP 60/65).

According to yet further features in preferred embodiments of the invention described below the immune response is selected from the group consisting of Th1 type cytokines expression, Th2 type cytokines expression, and T-cell proliferation.

According to another aspect of the present invention there is provided an article of manufacture, packaged and identified for use in the prevention and/or treatment of a vascular condition in a subject in need thereof. The article of manufacture includes a packaging material and a mucosal tolerance-inducing amount of an active ingredient selected from the group consisting of beta₂-glycoprotein-1 (β₂GPI)-derived peptide, and the packaging material includes a label or package insert indicating that the mucosal tolerance-inducing amount of the active ingredient is for prevention and/or treatment of a vascular condition in the subject via mucosal administration.

According to further features in preferred embodiments of the invention described below the at least one β₂GPI-derived peptide is a human β₂GPI-derived peptide.

According to yet further features in preferred embodiments of the invention as described below, the at least one β₂GPI-derived peptide is a synthetic peptide.

According to yet further features in preferred embodiments of the invention as described below, the at least one β₂GPI-derived peptide has a sequence as set forth in one of SEQ ID NOs: 25-57,315.

According to yet further features in preferred embodiments of the invention as described below, the combination of at least two β₂GPI-derived peptides is a mixture of peptides.

According to still further features in preferred embodiments of the invention as described below, the combination of at least two β₂GPI-derived peptide is a chimeric peptide comprising at least two β₂GPI-derived peptides in covalent linkage.

According to yet further features in preferred embodiments of the invention as described below, the chimeric peptide comprises a first β₂GPI-derived peptide having a sequence as set forth in one of SEQ ID NOs: 25-57,315 covalently linked to a second β₂GPI-derived peptide having a sequence as set forth in any of SEQ ID NOs: 25-57,315.

According to further features in preferred embodiments of the invention described below the β₂GPI is human β₂GPI.

According to yet further features in preferred embodiments of the invention described below the vascular condition is selected from the group consisting of atherosclerosis, cardiovascular disease, cerebrovascular disease, peripheral vascular disease, stenosis, restenosis and/or in-stent-stenosis.

According to still further features in preferred embodiments of the invention described below the article of manufacture further includes a therapeutically effective amount of at least one additional compound selected from the group consisting of HMGCoA reductase inhibitors (statins), mucosal adjuvants, corticosteroids, anti-inflammatory compounds, analgesics, growth factors, toxins, and additional tolerizing antigens.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates inhibition of early atherogenesis in apo-E deficient mice by nasal tolerance induced by administration of low doses of plaque associated molecules. 9-13 week old apo-E deficient mice were exposed intranasally, with mild sedation, to 3 doses of 10 μg/mouse each HSP 65 (HSP-65)(n=12), human oxidized LDL (H-oxLDL)(n=14), human β₂GPI (β₂GPI)(n=13), bovine serum albumin (BSA) or sham exposure to saline (PBS)(n=12). All mice received the atherogenic “Western” diet following last exposure. Atherogenesis is expressed as the area of atheromatous lesions in the aortic sinus 5 weeks following the 3^{rd} exposure.

FIG. 2 illustrates superior inhibition of early atherogenesis in apo-E deficient mice by mucosal tolerance induced by intranasal exposure to exceedingly low doses of HSP 65. Nasal tolerance was induced in 12-16 week old apo-E deficient mice by intranasal administration of 3 doses of 11 g/mouse HSP65 (HSP-65 low)(n=16) or 10 μg/mouse HSP65 (HSP-65 high)(n=14) every other day for 5 days. Control mice were exposed intranasally to an identical volume (10 μl) of bovine serum albumin, 10 μg/mouse (BSA)(n=14), or sham exposure to PBS (PBS)(n=14). All mice received the atherogenic “Western” diet following last exposure. Atherogenesis is expressed as the area of atheromatous lesions in the aortic sinus 5 weeks after the last nasal exposure.

FIG. 3 illustrates superior suppression of immune reactivity to atheroslerotic plaque antigens induced by nasal exposure to human β₂GPI. 5 week old male apo-E deficient mice were exposed intranasally to 10 μg/mouse human β₂GPI (H-b2-nt)(n=3); or alternately fed, by gavage, with 100 μg/mouse human β₂GPI (H-b2-ot)(n=3) in 0.2 ml PBS; or fed PBS alone (PBS)(n=3) every other day for 5 days. One week following the last feeding the mice were sensitized with a single subcutaneous injection of 10 μg/mouse human β₂GPI in 0.1 ml volume. Ten days later T-cells from inguinal lymph node were prepared as described in Materials and Methods section that follows, and exposed to the sensitizing human β₂GPI antigen for in-vitro assessment of proliferation. Proliferation, indicating immune reactivity, is expressed as the ratio between incorporation of labeled thymidine into the T-cell's DNA in the presence and absence of human β₂GPI antigen (stimulation index, S.I.).

FIG. 4 is a histogram illustrating the inhibition of atherogenesis in LDL RD mice by mucosal administration of β₂GPI. Human (H-β₂GPI) and bovine (B-β₂GPI) (50 or 500 μg) β₂GPI, BSA (500 μg) or PBS were administered orally (by gavage, as described in the Examples section hereinbelow) to LDL-receptor deficient mice (16-17 mice per group) and a “Western” (atherogenic) diet was then commenced for 4 weeks. Atherosclerotic lesion size (mm²) was determined at the aortic sinus. *p<0.001 as compared with BSA fed; **p<0.0001 as compared with BSA fed. Note the highly significant inhibition of atherogenesis in all groups receiving β₂GPI (40-50%).

FIGS. 5a-5d are a series of photomicrographs showing representative oil-red O stained sections through the upper sections of the aorta from LDL-RD mice receiving oral administration of β₂GPI (5C-bovine β₂GPI, 50 μg/mouse; 5D, human β₂GPI, 50 μg/mouse), BSA (5B, 500 μg/mouse) or PBS (5A). Upon sacrifice, the hearts and upper aorta were removed from all mice, embedded in OCT medium, frozen and sectioned as described hereinbelow. Note the marked reduction in fatty streak lesions (staining red) in the aorta sections from the β₂GPI treated mice.

FIG. 6 is a histogram demonstrating the inhibition of progression of advanced atherosclerotic plaquing in Apo E KO mice. Human β₂GPI (50 μg/mouse, n=16) or PBS (n=16) was administered orally (by gavage) to 20 week old mice, as described hereinbelow. Atherosclerotic lesion size (μm²) was determined in cryosections of the aortic sinus at 16 weeks from first treatment. Note the >30% inhibition of atherosclerotic plaque progression from time of initiating treatment (time 0) in the human β₂GPI-fed mice.

FIGS. 7a-7b are histograms showing the inhibition by mucosal administration of β₂GPI of cellular immune responses to atheroma-associated antigens in LDL RD mice. Proliferation of lymph-node cells from mice receiving oral administration of bovine β₂GPI (gray bars) or BSA (hatched bars) was assessed in vitro by thymidine uptake in the presence of different concentrations of β₂GPI (7A). Proliferation of lymph-node cells from mice immunized against oxLDL in addition to receiving oral administration of bovine β₂GPI (gray bars) or BSA (hatched bars) was assessed in vitro by thymidine uptake in the presence of different concentrations of oxLDL (7B). Thymidine uptake is expressed as the Stimulation Index. Note the marked suppression of cellular immune response to both β₂GPI and oxLDL stimulation conferred by oral administration of β₂GPI. *p<0.05.

FIG. 8 is a histogram showing the induction of anti-inflammatory Th2 cytokines by oral administration of β₂GPI. Conditioned medium was collected from lymph node cells of mice orally tolerized with β₂GPI (hatched bars) or BSA (solid bars), immunized with β₂GPI and incubated with β₂GPI (10 μg/ml) for 48 h. Levels of IL-4 and IL-10 were detected in the medium employing a capture ELISA kit as described in the Examples section hereinbelow. Note the remarkable increase in anti-inflammatory cytokines (IL-10 and IL-4) in cells from β₂GPI-tolerized mice. *p<0.01.

FIG. 9 is a photograph of RT-PCR products demonstrating the induction of an anti-inflammatory response in aortic tissue from mice tolerized with β₂GPI. Aorta tissue from 7-9 week old ApoE KO mice receiving oral administration (by gavage, as described hereinbelow) of β₂GPI (100 μg) or PBS, every other day for 10 days was removed, and RNA was prepared as described. Expression of cytokines IL-10 and IFN-γ, and the housekeeping gene β-actin, was measured by RT-PCR, using specific oligo primers. The upper panel is a photograph of the ethidium bromide stained gel of PCR products showing the induction of anti-inflammatory IL-10 expression in the β₂GPI-tolerized mice. The middle panel is a photograph of the ethidium bromide stained gel of PCR products showing the suppression of pro-inflammatory IFN-γ transcription in the β₂GPI-tolerized mice. Note the lack of effect of mucosal administration of β₂GPI on overall transcription rate, as evidenced by the unchanged levels of β-actin transcription (lower panel).

FIG. 10 is a histogram illustrating the inhibition of atherogenesis in LDL RD mice by mucosal administration of β₂GPI-derived peptides. Human β₂GPI-derived peptides S-1 (SEQ ID NO: 11), S-2 (SEQ ID NO: 12), S-3 (SEQ ID NO: 13) and S-4 (SEQ ID NO: 14), Human β₂GPI (H-β₂GPI) (100 μg), BSA (100 μg) in 0.2 ml PBS or PBS alone were administered orally (by gavage, as described in the Examples section hereinbelow) to LDL-receptor deficient mice (11-12 mice per group) and a “Western” (atherogenic) diet was then commenced for 5 weeks. Atherosclerotic lesion size (mm²) was determined at the aortic sinus. Note the significant inhibition with β₂GPI and all β₂GPI-derived peptides as compared with BSA and PBS controls (>44%); and the superior inhibition with S-4 and S-2 (>50%, p<0.001).

FIG. 11 is a histogram illustrating the effect on atherogenesis in LDL RD mice of immunization with β₂GPI-derived peptides. Human β₂GPI-derived peptides S-1 (SEQ ID NO: 11), S-2 (SEQ ID NO: 12), S-3 (SEQ ID NO: 13) and S-4 (SEQ ID NO: 14), Human β₂GPI (H-β₂GPI) (20 μg), BSA (20 μg) in 0.2 ml PBS or PBS alone, emulsified with 0.1 ml incomplete Freunds adjuvant were administered subcutaneously (as described in the Examples section hereinbelow) to LDL-receptor deficient mice (11-12 mice per group) in 4 immunizations every two weeks, and a “Western” (atherogenic) diet was then commenced for 5 weeks. Atherosclerotic lesion size (μm²) was determined at the aortic sinus. Note the great differences between extent of sinus lesion in the β₂GPI-derived peptide-treated mice.

FIG. 12 illustrates inhibition of early atherogenesis in apo-E deficient mice by mucosal tolerance induced by oral administration of β₂GPI-derived peptide. 10-11 week old Apo-E deficient mice were exposed orally (by gavage, as described in the Examples section hereinbelow) to 5 doses of 50 μg/mouse of human β₂GPI-derived peptide S-4 (SEQ ID NO: 14)(n=13), (β₂GPI)(n=13), or sham exposure to saline (PBS)(n=15). All mice received the atherogenic “Western” diet following last exposure. Atherogenesis is expressed as the area of atheromatous lesions in the aortic sinus 8 weeks following the fifth exposure. Note the significantly greater inhibition of atherosclerosis by oral administration of S-4 (>50%).

FIG. 13 illustrates inhibition of early atherogenesis in apo-E deficient mice by mucosal tolerance induced by nasal administration of β₂GPI-derived peptide. 10-11 week old Apo-E deficient mice were exposed nasally, with mild sedation, to 3 doses of 10 μg/mouse of human β₂GPI-derived peptide S-4 (SEQ ID NO: 14)(n=15), (β₂GPI)(n=15), or sham exposure to saline (PBS)(n=14). All mice received the atherogenic “Western” diet following last exposure. Atherogenesis is expressed as the area of atheromatous lesions in the aortic sinus 8 weeks following the third exposure.

FIG. 14 is a histogram showing superior inhibition by mucosal administration of β₂GPI-derived peptides of cellular immune responses to atheroma-associated antigens in LDL RD mice. Proliferation of lymph-node cells from mice immunized against oxLDL receiving oral administration (in 3 doses) of 100 μg of β₂GPI-derived peptides S-1 (SEQ ID NO: 11), S-2 (SEQ ID NO: 12), S-3 (SEQ ID NO: 13) and S-4 (SEQ ID NO: 14) or Human β₂GPI (H-β₂GPI) in 0.2 ml PBS or PBS alone or BSA was assessed in vitro by thymidine uptake in the presence of oxLDL. Thymidine uptake is expressed as the Stimulation Index. Note the superior suppression (>90%) of cellular immune response to oxLDL stimulation conferred by oral administration of β₂GPI-derived peptides S-3 and S-4.

FIG. 15 is a histogram showing significant inhibition by mucosal administration of β₂GPI-derived peptides of cellular immune responses to atheroma-associated antigens in LDL RD mice. Proliferation of lymph-node cells from mice immunized against oxLDL receiving oral administration (in 5 doses) of 100 μg of β₂GPI-derived peptides S-4-1 (SEQ ID NO: 15), S-4-3 (SEQ ID NO: 17), S-4-5 (SEQ ID NO: 19), S-4-6 (SEQ ID NO: 20), S-4-7 (SEQ ID NO: 21), S-4-8 (SEQ ID NO: 22), S-4-9 (SEQ ID NO: 23) or S-4-10 (SEQ ID NO: 24) in 0.2 ml PBS, or PBS alone was assessed in vitro by thymidine uptake in the presence of oxLDL. Only the response to S-4-derived peptide S-4-4 is shown. Thymidine uptake is expressed as the Stimulation Index. Note the significant suppression (nearly 50%) of cellular immune response to oxLDL stimulation conferred by oral administration of β₂GPI-derived peptide S-4-4.

### DESCRIPTION OF THE PREFERRED EMBODIMENTS

The present invention is of methods and compositions employing beta₂-glycoprotein-1 (β₂GPI)-derived peptides effective in inducing mucosal tolerance to atheroma related antigens, thus inhibiting inflammatory processes contributing to atheromatous vascular disease and sequalae.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Experimental and clinical evidence indicates a causative role for atheroma plaque-associated antigens in the etiology of the excessive inflammatory response in atherosclerosis. Both cellular and humoral immune reactivity to the plaque associated molecules oxidized LDL, β₂GPI and HSP 65 have been demonstrated, suggesting an important anti-oxidized LDL auto-immune component in atherogenesis. Thus, oxidized LDL, β₂GPI and HSP 65, and components thereof, have been the targets of numerous therapies for prevention and treatment of heart disease, cerebral-vascular disease and peripheral vascular disease.

Prior art teaches the application of plaque-associated antigens for detection and diagnosis of atherosclerosis and other plaque- and thrombosis related conditions. For example, Holvoet (U.S. Pat. No. 6,309,888) teaches the use of stage specific plaque associated antigens oxLDL and MDA-LDL for screening for Coronary Artery disease. Similarly, others (U.S. Pat. Nos. 5,472,883, 5,506,110, 5,900,359, and 5,998,223 and U.S. patent application Ser. No. 10/488,688 to Matsuura, et al, U.S. Pat. No. 5,344,758 to Krilis, et al, U.S. Pat. No. 5,750,309 to Wilson et al, U.S. patent application Ser. No. 10/492,479 to Koike et al, and Iverson et al., Immunology 1998; 95:15542-46) have disclosed the use of anti β₂GPI antibodies, to screen for serum indicators of APLS, SLE, cerebral infarct and atherosclerosis. The abovementioned disclosures propose diagnostic applications alone, and fail to recognize the therapeutic potential of these plaque associated molecules.

Although the role of immune response in the etiology and progression of atherosclerosis and other plaque related diseases remains controversial (see Meir, K, et al, International Atherosclerosis Soc. 2001 Commentary), many immune-based therapies have been proposed for atherosclerosis. General methods of reducing immune response in inflammatory and hyperreactive conditions are taught in, for example U.S. Pat. Nos. 6,277,969; 5,698,195 and 5,656,272 to Le at al, and 6,224,902 to Alving, et al, International Patent Application Nos. 001 001 2514 to Shurkovitz et al and 20010051156 A1 to Zeng. However, the proposed reduction or removal of mediators of immune reactivity, such as cytokines, tumor necrosis factor (TNF) and other pathogenic factors requires ongoing costly and potentially dangerous methods such as immunoadsorption of blood and prolonged anti-cytokine administration. Furthermore, no application to treatment of atherosclerosis or plaque-related disease is disclosed.

Specific immunotherapy with atheroma plaque-associated antigens has also been proposed. Bumol, et al, Calenoff, et al and Takano, et al (U.S. Pat. Nos. 5,196,324; 6,025,477 and 5,110,738, respectively) disclose the use of crude, poorly defined fractionated plaque preparations for immunization, monoclonal Ab preparation, diagnosis and treatment of atherosclerosis. These antigens, protein and lipid fractions of atheromatous tissue, are poorly defined, impractical for therapeutic use, and potentially hazardous in prolonged treatment.

Prior art teaches immunotherapy directed against atheroma-associated antigens. Zhou, et al (Arterioscler Thromb Vasc Biol, 2001; 21:108) achieved a significant reduction in early plaque formation in mice following footpad immunization with homogenized plaque or homologous MDA LDL. Palinski et al (PNAS USA 1995; 92:821-25) produced similar levels of protection in rabbits immunized with oxidized LDL. However, application of conventional immunization techniques to atheroma plaque components is problematic, since the adjuvant preparations required for immunization and boosters have produced accelerated plaque formation in similar regimen of immunization. Furthermore, relatively high doses (100 μgram/mouse/injection) of plaque antigen were required for immunity. Mucosal administration and induction of tolerance were not mentioned.

Recent animal and in-vitro studies with β₂GPI and other components of anticardiolipin and antiphospholipid antigens (see George J, et al Rheum Dis Clin North Am 2001; 27:603-10; Brey, et al Stroke 2001; 32:1701-06; Kyobashi, et al J Lipid Res 2001; 42:697-709; Koike T, et al Ann Med 2000; 32, Suppl. 1:27-31, Cabral A R et al Am J Med 1996; 101:472-81, Bili et al. Circulation 2000; 102:1258-; Altman, R. Thrombosis Journal 2003; 1:4, pgs 1-11; Segovia, J of Rheumatology; Hatori et al. Arthritis Rheum. 2000; 43:65-75; and Peirangeli et al J Autoimmunity 2004; 22:217-25, all of which are incorporated herein by reference, as if fully set forth) have demonstrated the association of β₂GPI with antiphospholipid syndrome, thrombosis, stroke, APLS, atherosclerosis and myocardial infarction. Although cryptic epitopes of the protein were implicated in humoral and cellular immune response, none of the abovementioned studies demonstrated protective immunity with the protein. Similarly, studies with HSP 65 (Birnie DH Eur Heart J 1998; 19:366-67; Xu Q, et al Circulation 1999; 100:1169-74; and Gromadzka G, et al Cerebrovasc Dis 2001; 12:235-39) have implicated this plaque associated antigen in stroke and heart disease, suggesting that humoral immunity may be a triggering factor.

However, numerous studies which fail to demonstrate a correlation between titers of anti-β₂GPI antibodies and recurrent or other cardiac disorders (see, for example, Bili, et al. Circulation, 2000; 102:1258-; Limaye et al Aust and New Zeal J of Med, 1999; 29; Levine et al JAMA, 2004; 291:576-84; Erkkila et al Atherosclerosis 2000; 20:204-9; Manzi, Rheumatology 2000; 39:353-359; Sadovsky, Amer Fam Phys December 1999) confound the understanding of the association of β₂GPI with vascular and cardiac events.

The complexity of atheroma plaque antigen immunity in atherosclerosis was demonstrated by Schoenfeld Y, et al (Autoimmunity 2000; 15:199-202), and George et al (Circulation, 1998; 98:1108-1115), who immunized LDL-receptor deficient (KO) mice with both β₂GPI and HSP 65 protein antigens, producing strong cellular and humoral responses, and surprisingly enhanced plaque formation. Similar increased atherogenesis was observed with passive transfer of β₂GPI activated lymphocytes (George et al. Circulation 2000; 102:1822-27). None of the above mentioned studies demonstrated inhibition of atherogenic processes by immune tolerance.

Suppression of immune response to autoantigens in atherosclerosis and related disease has been recently investigated. Victoria et al (U.S. Pat. Nos. 6,410,775, 6,207,160 and 5,844,409), Coutts et al (U.S. patent application Ser. No. 10/081,076), disclose specific non-immunogenic β₂GPI peptides lacking T cell epitopes for reducing antibody binding of immune cells and inducing B-cell tolerance in APLS, SLE and other diseases. However, no actual protection was demonstrated, and the disclosures emphasize the diagnostic use of the non immunogenic peptides. George J, et al (Atherosclerosis 1998; 138:147-52) has demonstrated the feasibility of immune suppression by hyperimmunization with MDA LDL and reduction of atherogenesis in mice. However, impractically large doses of antigen were required, and the paradoxical response to immunization with plaque antigens obviates the clinical efficacy of such therapy. Furthermore, none of the abovementioned studies disclose induction of mucosal tolerance for treatment of atherosclerosis.

Oral and mucosal tolerance for suppression and prevention of inflammatory conditions is well known in the art. For example, Weiner et al. have disclosed therapy, for the treatment of rheumatoid arthritis by mucosal administration of collagen and collagen peptides (U.S. Pat. Nos. 5,399,347; 5,720,955; 5,733,542; 5,843,445; 5,856,446; and 6,019,975), treatment of Type I diabetes by mucosal administration of insulin (U.S. Pat. Nos. 5,643,868; 5,763,396; 5,843,445; 5,858,968; 6,645,504; and 6,703,361) or glucagon (U.S. Pat. No. 6,645,504), uveoretinitis by mucosal administration of toleragens (U.S. Pat. No. 5,961,977), and multiple sclerosis by mucosal administration of myelin basic protein (MBP) (U.S. Pat. Nos. 5,849,298; 5,858,364; 5,858,980; 5,869,093; 6,077,509). Additional candidate conditions, antigens and modes of treatment by mucosal tolerance have been disclosed in U.S. Pat. Nos. 6,812,205, 5,935,577; 5,397,771; 4,690,683 to Weiner et al., U.S. Pat. No. 6,790,447 to Wildner et al; International Patent Nos. EP 0886471 A1, WO 01821951 to Haas, et al, U.S. Pat. No. 5,843,449 to Boots et al. (HCgp-39 for arthritis), and U.S. patent application Ser. No. 10/437,404 to Das (mucosal tolerance and relief from Crohn's disease by administration of Colonic Epithelial Protein).

U.S. patent application Ser. No. 09/806,400 to Shoenfeld et al filed Sep. 30, 1999, which is incorporated herein in its entirety, teaches the oral administration of plaque associated antigens for the induction of tolerance in LDL receptor deficient mice. Measuring arterial fatty streak lesion density, the inventors demonstrated that oral administration of oxidized LDL, β₂GPI and HSP 65 derived from animal sources were each able to produce approximately 30% reduction in atherogenesis. Additional evidence for the efficacy of mucosal tolerance with atheroma-associated antigen is provided in U.S. patent application Ser. Nos. 10/989,724, filed Nov. 17, 2004, 10/451,370, filed Jul. 2, 2003, 09/944,592, filed Sep. 4, 2001, and U.S. patent application Ser. No. 09/806,400, filed Mar. 30, 2001, (which are incorporated herein by reference, as if fully set forth).

While reducing the present invention to practice, the present inventors have uncovered that mucosal administration of β₂GPI-derived peptides results in the induction of mucosal tolerance, suppression of anti-β₂GPI and anti-oxLDL related immune reactivity and protection from atherosclerosis. Mucosal tolerance according to the invention is an advantageous method for treating vascular disorders for several reasons:

(1) Absence of toxicity: no toxicity has been observed in clinical trials or animal experiments involving oral or other mucosal administration of protein antigens, such as bovine myelin [which contains myelin basic protein (MBP) and proteolipid protein (PLP)] to humans afflicted with multiple sclerosis, or oral or by-inhalation administration of chicken Type II collagen to humans or rodents afflicted with rheumatoid arthritis [or a corresponding animal model disorder]; or oral administration of bovine S-antigen to humans afflicted with uveoretinitis; or oral administration of insulin to healthy volunteers.

(2) Containment of immunosuppression. Conventional treatments of immune system disorders involve administration of non-specific immunosuppressive agents, such as the cytotoxic drugs methotrexate, cyclophosphamide (CYTOXAN.RTM., Bristol-Myers Squibb), azathioprine (IMURAN.RTM., Glaxo Wellcome) and cyclosporin A (SANDIMMUNE.RTM., NEORAL.RTM., Novartis). Steroid compounds such as prednisone and methylprednisolone (also non-specific immunosuppressants) are also employed in many instances. All of these currently employed drugs have limited efficacy (e.g., against both cell-mediated and antibody-mediated autoimmune disorders). Furthermore, such drugs have significant toxic and other side effects and, more important, eventually induce “global” immunosuppression in the subject being treated. Prolonged treatment with the drugs down-regulates the normal protective immune response against pathogens, thereby increasing the risk of infection. In addition, patients subjected to prolonged global immunosuppression have an increased risk of developing severe medical complications from the treatment such as malignancies, kidney failure and diabetes.

(3) Convenience of therapy. Mucosal administration is more convenient than parenteral, or other forms, of administration.

(4) Greatly reduced incidence of alteration of the tolerizing molecule by digestive and metabolic processes (especially in non-oral routes of administration). These advantages provide superior protection from atherogenic processes, improved patient compliance and reduced cost of therapy.

(5) β₂GPI-derived peptides have greater specificity of action and potential for synthetic and/or recombinant production than the entire β₂GPI polypeptide.

(6) Combined β₂GPI-derived peptides or chimeric peptides afford possible synergy of action of different β₂GPI-derived peptide subtypes.

Thus, according to one aspect of the present invention there is provided a method for prevention and/or treatment of a vascular condition in a subject in need thereof. The method, according to this aspect of the present invention is effected by administering to a mucosal surface of a subject (e.g., a human) a mucosal tolerance-inducing amount of an antigenic portion of an active ingredient selected from the group consisting of beta₂-glycoprotein-1 (β₂GPI)-derived peptide, thereby inducing mucosal tolerance.

As used herein, the phrase “mucosal surface” is defined as a portion of the anatomy having exposed mucosal membranes having component or components of the mucosal associated lymphatic tissue. As used herein, the phrase “mucosal administration” is defined as application of any and all compounds and/or compositions to at least one mucosal surface. Non-limiting examples of mucosal administration are buccal, intranasal, otic (middle ear), conjunctival, vaginal, rectal, etc. Mucosal administration excludes, for example, intravenous, subcutaneous and epidural administration.

In preferred embodiments of the present invention, mucosal tolerance is effected by administering to a mucosal surface of the subject a mucosal tolerance-inducing amount of an active ingredient selected from the group consisting of beta₂-glycoprotein-1 (β₂GPI)-derived peptide. β₂GPI proteins have been identified in many phylogenetically diverse species, and peptides derived from β₂GPI protein suitable for use in the present invention include, but are not limited to, peptides derived from the following β₂GPI (also known as Apolipoprotein H, Apo-H, Activated protein C-binding protein, APC inhibitor, AntiCL cofactor) amino acid sequences:

Human β₂GPI precursor-GenBank Accession No. P02749, Human Apo-H precursor-GenBank Accession No. NP000033 (SEQ ID NO: 10), NBHU, Canis familiaris Apo-H GenBank Accession No. NP001002858, precursor GenBank Accession No. JN0465, Bos Taurus Apo-H-GenBank Accession No. NP776417, precursor GenBank Accession No. NBBO, Mus musculus Apo-H GenBank Accession No. NP038503, CAA72190; precursor GenBank Accession No. NBMS, Rattus norvegicus Apo-H precursor GenBank Accession No. NBRT, β₂GPI precursor Human GenBank Accession No. AAH26283, β₂GPI precursor Human GenBank Accession No. AAH20703, β₂GPI precursor Pan troglodytes GenBank Accession No. Q95LBO, β₂GPI precursor bovine GenBank Accession No. P17690, β₂GPI precursor Rat, GenBank Accession No. P26644, β₂GPI precursor Canis fam. GenBank Accession No. P33703, β₂GPI precursor Mus musc. GenBank Accession No. Q01339, β₂GPI Mus musc GenBank Accession No. BAA00945, CAA69701, AAB30789, β₂GPI Apo H Human GenBank Accession No. AAP72014, β₂GPI Human GenBank Accession No. CAA76845, CAA72279, CAA37664, CAA40977, CAA41113, AAB21330, and β₂GPI bovine GenBank Accession No. CAA42669. In a further preferred embodiment, the β₂GPI-derived peptides are peptides derived from human β₂GPI (SEQ ID NO: 10).

As used herein the phrase “β₂GPI-derived peptides” refers to peptides as this term is defined herein, e.g., cleavage products of β₂GPI, synthetic peptides chemically synthesized to correspond to the amino acid sequence of β₂GPI, peptides similar (homologous) to β₂GPI, for example, peptides characterized by one or more amino acid substitutions, such as, but not limited to, permissible substitutions, provided that at least 70%, preferably at least 75%, more preferably at least 80%, yet more preferably at least 85%, still more preferably at least 90% similarity is maintained, and functional homologues thereof. The terms “homologues” and “functional homologues” as used herein mean peptides with any insertions, deletions and substitutions which do not affect the biological activity of the peptide.

As used herein, the phrase “β₂GPI-derived peptides and combinations thereof” also refers to the abovementioned peptides in combination with one another. As used herein, the phrase “combination thereof” is defined as any of the abovementioned peptides, derived from β₂GPI, combined in a mixture and/or chimeric peptide with one or more additional, identical or non-identical peptides derived from β₂GPI. As used herein, the term “mixture” is defined as a non-covalent combination of peptides existing in variable proportions to one another, whereas the term “chimeric peptide” is defined as at least two identical or non-identical peptides covalently attached one to the other. Such attachment can be any suitable chemical linkage, direct or indirect, as via a peptide bond, or via covalent bonding to an intervening linker element, such as a linker peptide or other chemical moiety, such as an organic polymer. Such chimeric peptides may be linked via bonding at the carboxy (C) or amino (N) termini of the peptides, or via bonding to internal chemical groups such as straight, branched or cyclic side chains, internal carbon or nitrogen atoms, and the like. According to a preferred embodiment of the present invention, the chimeric peptide comprises a peptide derived from a β₂GPI-derived peptides as set forth in any of SEQ ID NOs: 25-57,315 linked via the carboxy (C) terminal with the amino (N) terminal of a peptide derived from β₂GPI as set forth in any of SEQ ID NOs: 25-57,315. It will be appreciated that, in further embodiments the chimeric peptides of the present invention can comprise all possible permutations of any of the peptides having an amino acid sequence as set forth in SEQ ID NOs: 25-57,315, covalently linked to any other of the peptides having an amino acid sequence as set forth in any of SEQ ID NOs: 25-57,315. Such chimeric peptides can be easily identified and prepared by one of ordinary skill in the art, using well known methods of peptide synthesis, including expression of recombinant proteins, and/or covalent linkage of peptides, from any of the large but finite number of combinations of peptides having an amino acid sequence as set forth in SEQ ID NOs: 25-57,315. It will be appreciated that, as used herein, the term “chimera” excludes any combination of peptides which yields a sequence identical to a peptide fragment of the native β₂GPI-derived peptides as set forth in any of SEQ ID Nos. 25-57,315. The peptides or chimeric peptides of the present invention may be produced by recombinant means or may be chemically synthesised by, for example, the stepwise addition of one or more amino acid residues in defined order using solid phase peptide synthetic techniques. Where the peptides or chimeric peptides may need to be synthesised in combination with other proteins and then subsequently isolated by chemical cleavage or alternatively the peptides or polyvalent peptides may be synthesised in multiple repeat units. The peptides or chimeric peptides may comprise naturally occurring amino acid residues or may also contain non-naturally occurring amino acid residues such as certain D-isomers or chemically modified naturally occurring residues. These latter residues may be required, for example, to facilitate or provide conformational constraints and/or limitations to the peptides or chimeric peptides. The selection of a method of producing the subject peptides or chimeric peptides will depend on factors such as the required type, quantity and purity of the peptides as well as ease of production and convenience.

The peptides or chimeric peptides of the present invention may first require their chemical modification for use in vivo. Chemical modification of the subject peptides or chimeric peptides may be important to improve their biological activity. Such chemically modified peptides are referred to herein as “analogues”. The term “analogues” extends to any functional chemical or recombinant equivalent of the peptides of the present invention, characterised, in a most preferred embodiment, by their possession of at least one of the abovementioned biological activities. The term “analogue” is also used herein to extend to any amino acid derivative of the peptides as described above.

Analogues of the peptides or chimeric peptides contemplated herein include, but are not limited to, modifications to side chains, incorporation of unnatural amino acids and/or their derivatives during peptide synthesis and the use of crosslinkers and other methods which impose conformational constraints on the peptides or their analogues.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2,4,6-trinitrobenzene sulphonic acid (TNBS); acylation of amino groups with succinic anhydride and tetrahydrophthalic anhydride; and pyridoxylation of lysine with pyridoxal-5′-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Examples of incorporating unnatural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. Modifications can be made to a β₂GPI-derived peptide for a variety of reasons, including 1) to reduce or eliminate an activity of a β₂GPI-derived peptide; 2) to enhance a property of a β₂GPI-derived peptide; 3) to provide a novel activity or property to a β₂GPI-derived peptide; or 4) to establish that an amino acid substitution that does or does not affect β₂GPI protein peptide activity. Modifications to a β₂GPI-derived peptide are typically made to the nucleic acid which encodes the β₂GPI-derived peptide, and can include deletions, point mutations, truncations, amino acid substitutions and additions of amino acids or non-amino acid moieties. Alternatively, modifications can be made directly to the peptide, such as by cleavage, addition of a linker molecule, addition of a detectable moiety (for example, biotin, fluorophore, radioisotope, enzyme, or peptide), addition of a fatty acid, and the like.

Modifications also embrace fusion proteins comprising all or part of the β₂GPI-derived peptide amino acid sequence. One of skill in the art will be familiar with methods for predicting the effect on protein conformation of a change in protein sequence, and can thus “design” a β₂GPI-derived peptide according to known methods. One example of such a method is described by Dahiyat and Mayo in Science 278:82-87 (1997), whereby proteins can be designed de novo. The method can be applied to a known protein to vary only a portion of the polypeptide sequence. By applying the computational methods of Dahiyat and Mayo, specific modifications of a β₂GPI-derived peptide can be proposed and tested to determine whether the modified β₂GPI-derived peptides retain a desired conformation.

β₂GPI-derived peptides include β₂GPI-derived peptides which are modified specifically to alter a feature of the polypeptide unrelated to its physiological activity. For example, cysteine residues can be substituted or deleted to prevent unwanted disulfide linkages. Similarly, certain amino acids can be changed to enhance expression of a β₂GPI protein peptide by eliminating proteolysis by proteases in an expression system (e.g., dibasic amino acid residues in yeast expression systems in which KEX2 protease activity is present).

Mutations of a nucleic acid which encode a β₂GPI-derived peptide preferably preserve the amino acid reading frame of the coding sequence, and preferably do not create regions in the nucleic acid which are likely to hybridize to form secondary structures, such as hairpins or loops, which can be deleterious to expression of the derivative polypeptide.

Mutations can be made by selecting an amino acid substitution, or by random mutagenesis of a selected site in a nucleic acid which encodes the polypeptide. Derivative β₂GPI-derived peptides are then expressed and tested for one or more activities to determine which mutation provides a derivative polypeptide with a desired property. Further mutations can be made to derivative (or to native β₂GPI-derived peptides) which are silent as to the amino acid sequence of the polypeptide, but which provide preferred codons for translation in a particular host. The preferred codons for translation of a nucleic acid in, e.g., E. coli, are well known to those of ordinary skill in the art. Still other mutations can be made to the noncoding sequences of a β₂GPI gene or cDNA clone to enhance expression of the polypeptide.

The activity of derivatives of β₂GPI-derived peptides can be tested by cloning the gene encoding the derivative or variant β₂GPI-derived peptide into a prokaryotic or eukaryotic (e.g., mammalian) expression vector, introducing the vector into an appropriate host cell, expressing the β₂GPI-derived peptide, and testing for a functional capability of the β₂GPI-derived peptides as disclosed herein. For example, the β₂GPI-derived peptide can be tested for its ability to bind to an anti-β₂GPI antibody, to elicit an immune response in a sensitized animal, or to suppress a vascular disorder, as set forth below in the examples.

The skilled artisan will also realize that conservative amino acid substitutions may be made in β₂GPI-derived peptides to provide functionally equivalent derivatives (functional equivalents) of the foregoing polypeptides, i.e., derivatives which retain the functional capabilities of the β₂GPI-derived peptides. As used herein, a “conservative amino acid substitution” refers to an amino acid substitution which does not alter the relative charge or size characteristics of the polypeptide in which the amino acid substitution is made. Derivatives can be prepared according to methods for altering polypeptide sequence known to one of ordinary skill in the art such as are found in references which compile such methods, e.g., Molecular Cloning. A Laboratory Manual, J. Sambrook, et al., eds., Second Edition, Cold Spring Harbor Laboratory Press, Cold Spring Harbor, N.Y., 1989, or Current Protocols in Molecular Biology, F M Ausubel, et al., eds., John Wiley & Sons, Inc., New York. Exemplary functionally equivalent derivatives of the β₂GPI-derived peptides include polypeptides having conservative amino acid substitutions of SEQ ID NO.10 (Human beta2GPI). Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

As used herein, the phrase “mucosal tolerance-inducing amount” of a β₂GPI-derived peptide is defined as the amount sufficient to stimulate a reduction in immune reactivity to β₂GPI, or a β₂GPI-derived peptide, which reduction in immune reactivity can also be associated with decreased stimulation index of lymph node cells, decreased cytokine production, inhibition of atherogenic processes in the recipient thereof, and the like, assessment of which is described in detail in the Examples section hereinbelow.

As used herein, the phrases “atheroma plaque related antigens” or “atheroma plaque-associated antigens” are defined as any and all protein, carbohydrate, lipid and nucleic acid molecules, portions thereof (antigenic portions), their derivatives, or combinations thereof physically or functionally related to the etiology, pathogenesis, symptomatology and/or treatment of a plaque-related condition or disease. Such molecules may be, for example, plaque components such as oxidized LDL, foam cell components, etc, but may also include humoral and cellular entities, such as antibodies, cytokines, growth factors and T cell receptors.

As used herein, the phrase “antigen” refers to a portion of a molecule capable of eliciting an immune response. For example, in cases where the molecule is a protein, a peptide or a polypeptide (e.g. β₂GPI-derived peptide) such a portion can include a stretch of 6-8 amino acids that constitute an antigenic epitope. Methods for predicting antigenic portions are well known in the art, for example, DNASTAR'S PROTEAN sequence analysis and prediction module (DNAStar, Madison, Wis.). As such determining antigenic portions of plaque-associated molecules suitable for use with the present invention is well within the capabilities of an ordinarily skilled artisan.

β₁GPI and β₂GPI-derived peptides (as well as fragments, analogs, portions and derivatives thereof) can be purified from natural sources (the tissue or organ where β₂GPI normally occurs) and can also be obtained using recombinant DNA technology, in bacterial, yeast, insect (e.g. baculovirus) and mammalian cells using techniques well-known to those of ordinary skill in the art.

As used herein the term “vascular disorder” refers to a disease or process involving tissue intrinsic to the blood vessels, particularly the arterial vessels, in which the lumen of affected vessels are narrowed as a result. The archetype of vascular disorder is atherosclerosis. A vascular disorder can involve vessels associated with one or more vascular beds, e.g., the coronary arteries, the cerebral arteries, the aorta, the renal arteries, the splanchnic bed, the peripheral arteries, etc. Included are arterial aneurysms, e.g., aortic aneurysm. Such aneurysms are preferably non-traumatic in origin and can but need not necessarily be atherosclerotic. Also included are a number of principally inflammatory vascular disorders, including but not limited to: allergic angiitis and granulomatosis (Churg-Strauss disease), Behget's syndrome, Cogan's syndrome, graft-versus-host disease (GvHD), Henoch-Schonlein purpura, Kawaski disease, leukocytoclastic vasculitis, polyarteritis nodosa (PAN), microscopic polyangiitis, polyangiitis overlap syndrome, Takayasu's arteritis, temporal arteritis, transplant rejection, Wegener's granulomatosis, and thromboangiitis obliterans (Buerger's disease).

Immune tolerance established using the present methodology can be used in the prevention and/or treatment of disorders associated with plaque formation, including but not limited to atherosclerosis, atherosclerotic cardiovascular disease, cerebrovascular disease, peripheral vascular disease, stenosis, restenosis and in-stent-stenosis. Some non-limiting examples of atherosclerotic cardiovascular disease are myocardial infarction, coronary arterial disease, acute coronary syndromes, congestive heart failure, angina pectoris and myocardial ischemia. Some non-limiting examples of peripheral vascular disease are gangrene, diabetic vasculopathy, ischemic bowel disease, thrombosis, diabetic retinopathy and diabetic nephropathy. Non-limiting examples of cerebrovascular disease are stroke, cerebrovascular inflammation, cerebral hemorrhage and vertebral arterial insufficiency. Stenosis is occlusive disease of the vasculature, commonly caused by atheromatous plaque and enhanced platelet activity, most critically affecting the coronary vasculature. Restenosis is the progressive re-occlusion often following reduction of occlusions in stenotic vasculature. In cases where patency of the vasculature requires the mechanical support of a stent, in-stent-stenosis may occur, re-occluding the treated vessel. The measurable symptoms and diagnostic markers of these vascular disorders are well established in the literature and known to physicians practicing in this field. See, for example, Harrison's Principles of Internal Medicine, 14th ed., A S Fauci et al., eds., New York: McGraw-Hill, 1998.

The methods of the present invention can be administered as a sole therapeutic and/or preventive treatment, or in conjunction with one or more additional treatments. Conventional treatment modalities for atherosclerosis, and other vascular conditions include, but are not limited to various anti-inflammatory, analgesic, and anti-coagulant agents well known in the art. Thus, according to a preferred embodiment of the present invention the β₂GPI-derived peptides are administered along with at least one additional compound selected from the group consisting of HMGCoA reductase inhibitors (statins), mucosal adjuvants, corticosteroids, anti-inflammatory compounds, analgesics, growth factors, toxins and additional tolerizing antigens. It will be appreciated by one of ordinary skill in the art, that the additional compounds or treatment regimen are administered in a conventional manner, and not to mucosal surfaces in a manner so as to induce mucosal tolerance thereto. In addition, it will be appreciated that use of the methods of the present invention does not preclude the initiation or continuation of other therapies for the abovementioned diseases or conditions, except where specifically counter-indicated.

HMGCoA reductase inhibitors that can be administered in combination with β₂GPI or derivative thereof include, but are not limited to, Pravastatin (PRAVACHOL, Bristol-Myers Squibb), Lovastatin (MEVACOR, Merck), Simvastatin (ZOCOR, Merck), Fluvastatin (LESCOL, Novartis), Atorvastatin (LIPITOR, Parke-Davis), Cerivastatin (BAYCOL, Bayer), Rosuvastatin (CRESTOR, Astra-Zeneca) and Lovastatin+extended release niacin (ADVICOR, Kos Pharmaceutical).

Anti-inflammatory drugs that can be administered in combination with the β₂GPI or derivative thereof of the present invention include, but are not limited to, Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lomoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium.

Analgesics that can be administered in combination with the β₂GPI or derivative thereof of the present invention include, but are not limited to, acetaminophen, salicylates, butalbital, narcotic analgesics such as fentynal and central analgesics such as tramadol.

Mucosal adjuvants that can be administered in combination with β₂GPI or derivative thereof are well known in the art (see, for example, U.S. Pat. Nos. 6,270,758 to Staats, et al and 5,681,571 to Holmgren et al.).

According to another preferred embodiment of the present invention, a combination of at least two of the abovementioned molecules is administered to the subject.

The method of the invention may be used for prevention and/or treatment of non-atherosclerosis related diseases. For example, β₂GPI-derived peptides, anti-β₂GPI-derived peptide antibodies, and β₂GPI in complex with phospholipids and phospholipid metabolites have been clearly implicated in the pathogenesis, and therefore potential treatment of additional, non-atherosclerosis-related diseases. Such diseases and syndromes include Anti Phospholipid Syndrome (APLS or APS) (Koike T, et al Ann Med 2000; 32 Suppl I:27-31), thrombosis, preeclampsia, acute otitis media, venous sinus thrombosis (Oestricher-Kedem et al. Laryngoscope 2004; 114:90-94), scleroderma (Sato et al. Ann Rheum Dis 2003; 62:771-74), atopic disease (Ambrozic et al Int Immunology 2002; 14:823-30), Systemic Lupus Erythematosus (SLE) (Davies, Rheumatology 2002; 41:395-400, and U.S. Pat. Nos. 5,344,758 and 6,207,160, to Krilis, et al and Victoria, et al, respectively), venous and arterial thromboses (Cabral A R, et al Am J Med 1996; 101:472-81) and others.

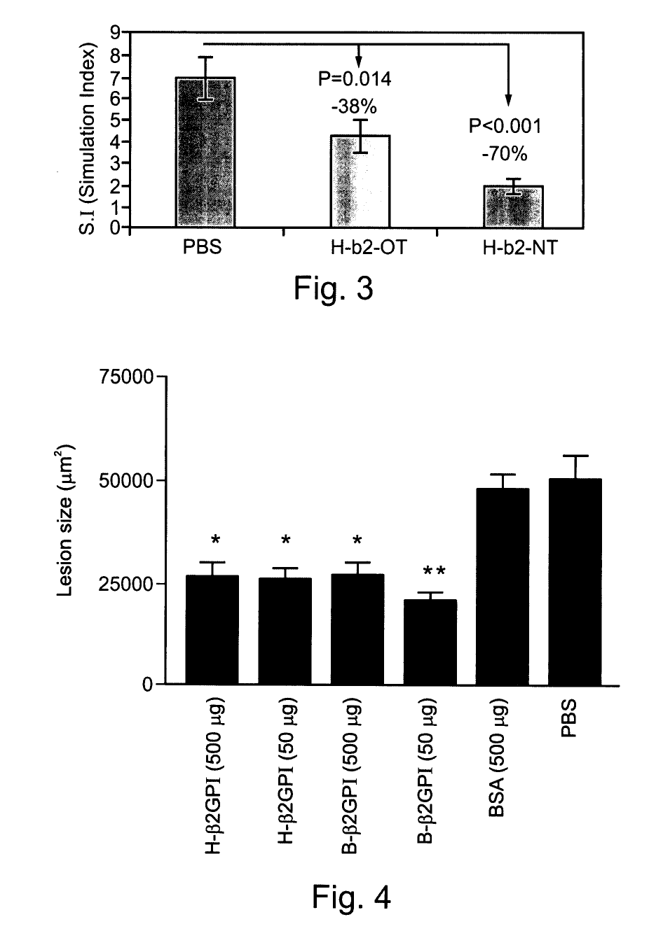

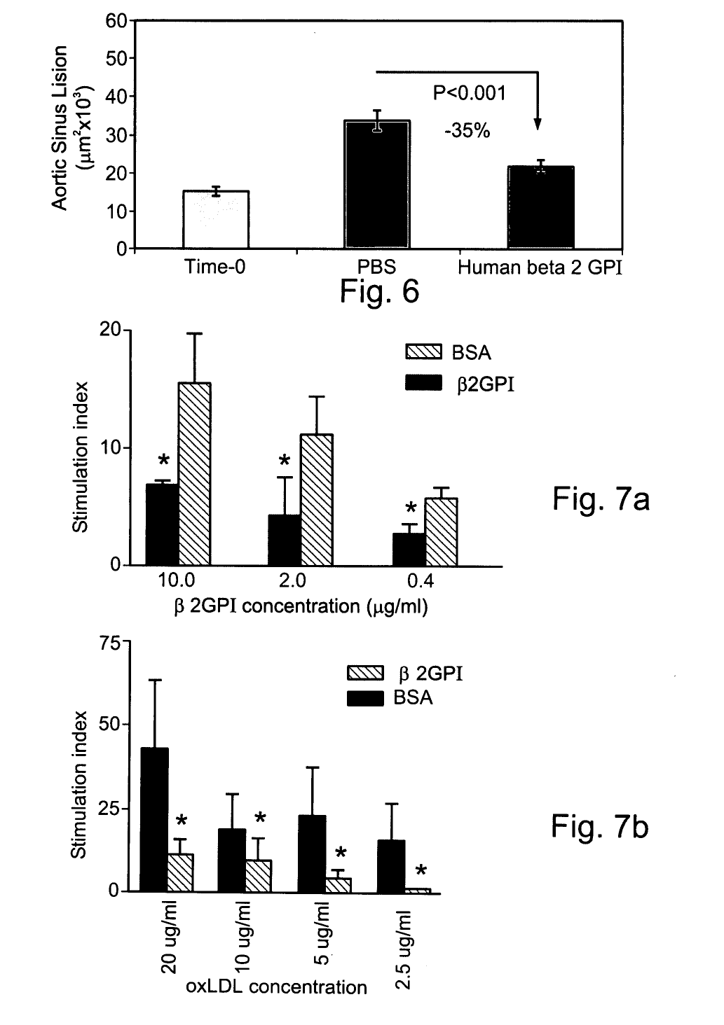

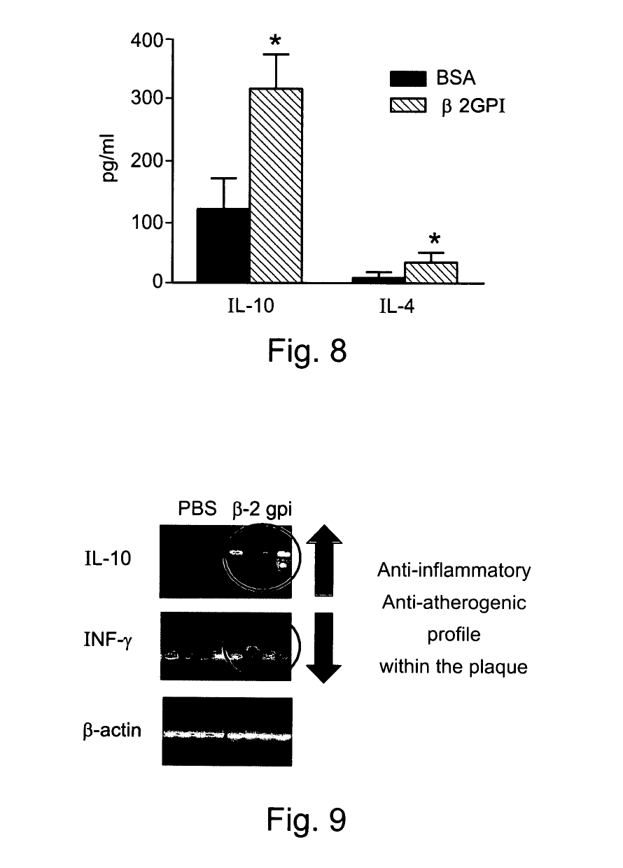

While reducing the present invention to practice, it was uncovered that mucosal administration of β₂GPI to LDL-RD mice reduces the T-cell response to stimulation by β₂GPI in previously sensitized animals. Examples 3 and 6 hereinbelow describe the reduction by up to 70% of the stimulation index of lymph node cells (FIGS. 3 and 7a-b), the induction of anti-inflammatory cytokines IL-10 and IL-4 (FIGS. 8 and 9), and the suppression of plaque-associated pro-inflammatory INF-γ cytokine expression (FIG. 9).

Thus, the methods of the present invention can be used alter an immune response to β₂GPI in a subject in need thereof. Thus, according to one aspect of the present invention there is provided a method for modulating an immune response to a β₂GPI in a subject in need thereof. The method, according to this aspect of the present invention is effected by administering to a mucosal surface of a subject (e.g., a human) a mucosal tolerance-inducing amount of at least one beta₂-glycoprotein-1 (β₂GPI)-derived peptide, thereby inducing mucosal tolerance and modulating the immune response to the β₂GPI.

In one preferred embodiment, the subject is at risk for, or suffering from a condition characterized by excess reactivity to β₂GPI, and the modulating is reducing the immune reactivity to β₂GPI in the subject. Both humoral and cellular immune reactivity can be readily assessed in-vitro and in-vivo, by one of ordinary skill in the art, according to art-recognized criteria, such as measurement of circulating antibodies, isotype antibodies, cytokine profile, stimulation index, and the like. Thus, according to a preferred embodiment, the immune response is selected from the group consisting of Th2 cytokine expression, Th1 cytokines expression, and T-cell proliferation. Exemplary methods for assessing immune reactivity are described in detail hereinbelow.

Further, while reducing the present invention to practice, it was unexpectedly uncovered that mucosal administration of β₂GPI and β₂GPI-derived peptides to LDL-RD mice inhibits not only the T-cell response to stimulation by β₂GPI in sensitized animals, but also the primary immune response to stimulation by other atheroma plaque-associated antigens, such as oxidized LDL (see FIGS. 7a and 7b, 14 and 15) in sensitized animals. Without wishing to be limited by a single hypothesis, this tolerizing effect on oxLDL responsiveness can be mediated through the “bystander effect”, involving regulatory cells secreting nonantigen-specific cytokines that suppress inflammation in the microenvironment where the mucosally administered antigen is localized. Thus, according to another aspect of the present invention, there is provided a method for modulating an immune response to an atheroma plaque-related antigen in a subject in need thereof. The method, according to this aspect of the present invention is effected by administering to a mucosal surface of a subject (e.g., a human) a mucosal tolerance-inducing amount of an antigenic portion of at least one beta₂-glycoprotein-1 (β₂GPI)-derived peptide as an active ingredient, thereby inducing mucosal tolerance and modulating the immune response to the atheroma plaque-related antigen. In one preferred embodiment, the at least one beta₂-glycoprotein-1 (β₂GPI)-derived peptide is a combination of at least two beta₂-glycoprotein-1 (β₂GPI)-derived peptides.

Atheroma plaque-related antigens are defined hereinabove. In one preferred embodiment, the atheroma plaque related antigens are selected from the group consisting of β₂GPI, oxidized LDL, and heat-shock protein (HS 60/65).

Suitable formulations according to the invention include formulations of β₂GPI-derived peptides or chimeric peptides adapted for oral, enteral, buccal, nasal, bronchial or intrapulmonary administration. It will be appreciated that the formulations and administration of the present invention from mucosal administration are selected to provide sufficient interaction between the tolerizing peptide and the mucosal associated lymphatic tissue (MALT), allowing the accumulation of tolerizing amounts of the β₂GPI-derived peptide in the MALT. These naturally exclude formulations for and administration by intramuscular, intravenous, intra-articular, intradermal, transdermal, subcutaneous and/or enteral methods, designed for systemic delivery of the active ingredient. The preparation of such formulations is well within the skill of the art. Thus, it is preferred that such formulations not contain substances that can act as adjuvants in order to avoid sensitization of the treated subject. It is also preferred that the antigens employed be of synthetic provenance and not isolated from biological sources to avoid the risk of infection (notably, but not exclusively, to avoid transmission of agent responsible for the Creutzfeld-Jacob disease). Additionally, it is preferred that the formulation not contain adsorption promoting agents or ingredients that protect against proteolytic degradation.

Suitable oral formulations for use in tolerization of T-cell mediated immune responses according to the present invention can be in any suitable orally administrable form, for example, a pill, a liquid, or a capsule or caplet containing an effective amount of antigen. Each oral formulation may additionally comprise inert constituents including pharmaceutically acceptable carriers, diluents, fillers, disintegrants, flavorings, stabilizers, preservatives, solubilizing or emulsifying agents and salts as is well-known in the art. For example, tablets may be formulated in accordance with conventional procedures employing solid carriers and other excipients well-known in the art. Capsules may be made from any 1 cellulose derivatives. Nonlimiting examples of solid carriers include starch, sugar, bentonite, silica and other commonly used inert ingredients. Diluents for liquid oral formulations can include inter alia saline, syrup, dextrose and water.

The antigens (i.e., β₂GPI-derived peptides, or chimeric peptides and therapeutically effective fragments and analogs thereof) used in the present invention can also be made up in liquid formulations or dosage forms such as, for example, suspensions or solutions in a physiologically acceptable aqueous liquid medium. Such liquid media include water, or suitable beverages, such as fruit juice or tea which will be convenient for the patient to sip at spaced apart intervals throughout the day. When given orally in liquid formulations the antigen may be dissolved or suspended in a physiologically acceptable liquid medium, and for this purpose the antigen may be solubilized by manipulation of its molecule (e.g., hydrolysis, partial hydrolysis or trypsinization) or adjustment of the pH within physiologically acceptable limits (e.g., 3.5 to 8). Alternatively, the antigen may be reduced to micronized form and suspended in a physiologically acceptable liquid medium, or in a solution.

Sustained release oral delivery systems are also contemplated and are preferred. Nonlimiting examples of sustained release oral dosage forms include those described in U.S. Pat. No. 4,704,295, issued Nov. 3, 1987; U.S. Pat. No. 4,556,552, issued Dec. 3, 1985; U.S. Pat. No. 4,309,404, issued Jan. 5, 1982; U.S. Pat. No. 4,309,406, issued Jan. 5, 1982; U.S. Pat. No. 5,405,619, issued Apr. 10, 1995; PCT International Application WO 85/02092, published May 23, 1985; U.S. Pat. No. 5,416,071, issued May 16, 1995; U.S. Pat. No. 5,371,109, issued Dec. 6, 1994; U.S. Pat. No. 5,356,635, issued Oct. 18, 1994; U.S. Pat. No. 5,236,704, issued Aug. 17, 1993; U.S. Pat. No. 5,151,272, issued Sep. 29, 1992; U.S. Pat. No. 4,985,253, issued Jan. 15, 1991; U.S. Pat. No. 4,895,724, issued Jan. 23, 1990; and U.S. Pat. No. 4,675,189, issued Jun. 23, 1987, incorporated as if fully set forth herein by reference.

Sustained release oral dosage forms coated with bioadhesives can also be used. Examples are compositions disclosed in European Published Application EP 516141; U.S. Pat. No. 4,226,848; U.S. Pat. No. 4,713,243; U.S. Pat. No. 4,940,587; PCT International Application WO 85/02092; European Published Application 205282; Smart J D et al. (1984) J Pharm Pharmacol 36:295-9; Sala et al. (1989) Proceed Intern Symp Control Rel Bioact Mater 16:420-1; Hunter et al. (1983) International Journal of Pharmaceutics 17:59-64; “Bioadhesion—Possibilities and Future Trends, Kellaway,” Course No. 470, May 22-24, 1989, incorporated as if fully set forth herein by reference.

Commercially available sustained release formulations and devices include those marketed by ALZA Corporation, Palo Alto, Calif., under tradename ALZET, INFUSET, IVOS, OROS, OSMET, or described in one or more U.S. Pat. No. 5,284,660, issued Feb. 9, 1994; U.S. Pat. No. 5,141,750, issued Aug. 25, 1992; U.S. Pat. No. 5,110,597, issued May 5, 1992; U.S. Pat. No. 4,917,895, issued Apr. 17, 1990; U.S. Pat. No. 4,837,027, issued Jun. 6, 1989; U.S. Pat. No. 3,993,073, issued Nov. 23, 1976; U.S. Pat. No. 3,948,262, issued Apr. 6, 1976; U.S. Pat. No. 3,944,064, issued Mar. 16, 1976; and U.S. Pat. No. 3,699,963; International Applications PCT/US93/10077 and PCT/US93/11660; and European Published Applications EP 259013 and EP 354742, incorporated as if fully set forth herein by reference.

Administration of the tolerizing β₂GPI-derived peptide antigen or chimeric peptides can also be affected by transforming cells of the mucosal tissue with a nucleic acid capable of encoding the antigen, and expression within cells of the mucosa. Methods for inducing mucosal immunity using local expression of nucleic acid constructs are disclosed in U.S. patent application Ser. No. 10/076,900 to Weiner et al, filed Feb. 4, 2004, incorporated as if fully set forth herein by reference.

Sustained release compositions and devices are suitable for use in the present invention because they serve to prolong contact between the antigen and the gut-associated lymphoid tissue (GALT) and thus prolong contact between the antigen and the immune system. In addition, sustained release compositions obviate the need for discrete multi-dose administration of the antigen and permit the required amount of antigen to be delivered to GALT in one or two daily doses. This is anticipated to improve patient compliance.

Orally administrable pharmaceutical formulations containing at least one β₂GPI-derived peptide are prepared and administered to mammals who have manifested symptoms of vascular disorder, such as atherosclerosis. Additionally, subjects who are at risk for developing a vascular disorder, i.e., have a genetic predisposition to developing the disorder, as determined through suitable means, such as genetic studies and analysis, are treated with similar oral preparations.

Pharmaceutical formulations for oral or enteral administration to treat vascular disorders are prepared from an at least one β₂GPI-derived peptide and a pharmaceutically acceptable carrier suitable for oral ingestion. The quantity of the β₂GPI-derived peptide in each daily dose may be between 0.001 mg and 1000 mg per day. However, the total dose required for treatment can vary according to the individual and the severity of the condition. This amount can be further refined by well-known methods such as establishing a matrix of dosages and frequencies of administration.

For by-inhalation administration (i.e., delivery to the bronchopulmonary mucosa) suitable sprays and aerosols can be used, for example using a nebulizer such as those described in U.S. Pat. No. 4,624,251 issued Nov. 25, 1986; U.S. Pat. No. 3,703,173 issued Nov. 21, 1972; U.S. Pat. No. 3,561,444 issued Feb. 9, 1971; and U.S. Pat. No. 4,635,627 issued Jan. 13, 1971, incorporated as if fully set forth herein by reference. The aerosol material is inhaled by the subject to be treated.

Other systems of aerosol delivery, such as the pressurized metered dose inhaler (MDI) and the dry powder inhaler as disclosed in Newman S P in Aerosols and the Lung, S W Clarke S W and D Davis, eds. pp. 197-224, Butterworths, London, England, 1984, can be used when practicing the present invention.

Aerosol delivery systems of the type disclosed herein are available from numerous commercial sources including Fisons Corporation (Bedford, Mass.), Schering Corp. (Kenilworth, N.J.) and American Pharmoseal Co. (Valencia, Calif.).

Formulations for nasal administration can be administered as a dry powder or in an aqueous solution. Preferred aerosol pharmaceutical formulations may comprise for example, a physiologically acceptable buffered saline solution containing at least one β₂GPI-derived peptide of the present invention.

Dry aerosol in the form of finely divided solid comprising at least one β₂GPI-derived peptide in particle form, which particles are not dissolved or suspended in a liquid are useful in the practice of the present invention. The antigen may be in the form of dusting powders and comprise finely divided particles having an average particle size of between about 1 and 5 μm, preferably between 2 and 3 μm. Finely divided antigen particles may be prepared by pulverization and screen filtration using techniques well known in the art. The particles may be administered by inhaling a predetermined quantity of the finely divided material, which can be in the form of a powder.

Specific non-limiting examples of the carriers and/or diluents that are useful in the pharmaceutical formulations of the present invention include water and physiologically acceptable buffered saline solutions such as phosphate buffered saline solutions pH 7.0-8.0.

The mucosally administered formulation of the present invention may include a thermosetting gel which increases in viscosity at body temperature upon contact with the mucosa.

Formulations for buccal administration can include mucoadhesive mixed with effective amounts of a β₂GPI-derived peptide and/or a therapeutically effective β₂GPI-derived peptide analog. Effective amounts are anticipated to vary according to the formulation employed. For formulation administered by inhalation, the effective amount is likely to be less than that of the oral dose.

Preferably, the duration of treatment in humans should be a minimum of two weeks, and typically three months, and may be continued indefinitely or as long as benefits persist. The treatment may be discontinued if desired (in the judgment of the attending physician) and the patient monitored for signs of relapse. If clinical symptoms or other disorder indicators show that the patient is relapsing, treatment may resume.

As will be understood by those skilled in the art, the dosage will vary with the antigen administered and may vary with the sex, age, and physical condition of the patient as well as with other concurrent treatments being administered. Consequently, adjustment and refinement of the dosages used and the administration schedules will preferably be determined based on these factors and especially on the patient's response to the treatment. Such determinations, however, require no more than routine experimentation, as illustrated in Examples provided below.

Administration of a β₂GPI-derived peptide can be conjoined with mucosal administration of one or more enhancers, i.e. substances that enhance the tolerizing effect of the a β₂GPI-derived peptide and/or a therapeutically effective β₂GPI-derived peptide analog antigen. Such enhancers include lipopolysaccharide (LPS), Lipid A (as described in U.S. application Ser. No. 08/202,677, published as WO 91/01333), IL-4, IL-10 and Type I interferon (See, e.g., U.S. application Ser. Nos. 08/420,980 and 08/420,979 and WO 95/27499 and WO 95/27500). Other suitable enhancers of mucosal tolerance are a group of β-1,3, β-1-6 glucan products described in detail in U.S. Patent Application Nos. 20030104010 and 20020009463 to Raa et al., and the oral tolerance inducing agents disclosed by Holmgren et al in U.S. Pat. No. 5,681,571, all incorporated as if fully set forth herein by reference) As used in the preceding sentence, “conjoined with” means before, substantially simultaneously with, or after administration of these antigens. Naturally, administration of the conjoined substance should not precede nor follow administration of the antigen by so long an interval of time that the relevant effects of the substance administered first have worn off. Therefore, enhancers should usually be administered within about 24 hours before or after the β₂GPI-derived peptide and/or a therapeutically effective β₂GPI-derived peptide analog antigen and preferably within about one hour.

As used herein the terms “therapeutically effective peptide” or “therapeutically effective peptide analog” refers to a peptide or polypeptide containing partial amino acid sequences or moieties of β₂GPI-derived peptide possessing the ability to treat a vascular disorder and/or modulate the immune response to a atheroma-derived antigen. Preferably, such fragments are able to suppress or prevent an inflammatory response upon mucosal administration. Such fragments need not possess all the immunogenic properties of the entire β₂GPI-derived peptide. By way of non-limiting example, when MBP is administered parenterally to susceptible mice in the presence of an adjuvant, it induces experimental allergic encephalomyelitis RAE). It is known that certain non-disease-inducing fragments of MBP (i.e., fragments of MBP which do not induce EAE when administered parenterally with an adjuvant) nevertheless possess autoimmune-suppressive activity when administered orally (or enterally) or in aerosol form to mammals suffering from EAE. Examples of such fragments are reported in U.S. patent application Ser. No. 07/065,734, filed Jun. 24, 1987, and International Patent Application No. PCT/US88/02139, filed Jun. 24, 1988. Similarly, the present inventors have found that while immunization with a single subcutaneous administration of β₂GPI and adjuvant induced atherogenesis in LDLR−/− mice (George, et al, Circulation, 1998; 98:1108-1115), mucosal administration of the same β₂GPI antigen resulted in reduced plaque formation and inhibition of development of atherosclerosis (see U.S. patent application Ser. No. 10/450,370 to Harats et al, filed Jan. 3, 2002, and PCT IL02/00005 to Harats et al., filed Jan. 3, 2002). Therapeutically effective peptides and peptide analogs can be identified by observing a change in cytokine release profile, such as illustrated in the Examples or in other in vitro or in vivo assays which are predictive of a human vascular disorder and from which agents can be selected which alleviate detectable symptoms of the disorder. Cytokines can be measured using routine assays, including commercially available immunoassays such as radioimmunoassay (RIA), enzyme-linked immunosorbent assay (ELISA) and RT-PCR.

As used herein the term “therapeutically effective derivative” of β₂GPI-derived peptides is defined as β₂GPI-derived peptides or their therapeutically effective fragments (e.g., inflammatory response-suppressive fragments) which possess the same biological activity, i.e., the ability to treat the condition, e.g., by eliminating or suppressing the inflammatory response, upon mucosal administration, either nasally, orally, or enterally. By way of non-limiting example, the term includes peptides having amino acid sequences which differ from the amino acid sequence of the β₂GPI-derived peptide or therapeutically effective peptides thereof by one or more amino acid residues (while still retaining the inflammatory response-suppressive activity of the β₂GPI-derived peptide) as well as compounds or compositions which mimic the inflammatory response-suppressive activity of the β₂GPI-derived peptide in its ability to suppress or alleviate the symptoms of the disorder.

The tolerance induced by the autoimmune-suppressive agents of this invention is dose-dependent. Dose dependency was also seen in the autoimmune arthritis system. Moreover, the mucosal administration of an irrelevant antigen (i.e., one not implicated in an autoimmune disease, such as ovalbumin (OVA) peptide, histone protein, or certain synthetic fragments of MBP) has no effect on the clinical manifestation of the autoimmune disease.

Various animal models have been developed for the study of atherosclerosis and are predictive of human atherosclerosis. Among the more common models are those in which inbred strains of mice have been rendered deficient for either the LDL receptor (LDLR −/−) or apolipoprotein E (apo E −/−) by a gene knockout. The LDL receptor is a 160 kDa glycoprotein responsible for the transfer of LDL out of the plasma and into the cytoplasm of virtually all cell types. The major site of LDL uptake and catabolism is the liver. LDLR−/− mice created on a C57BL/6 background develop accelerated atherosclerosis when fed a high cholesterol diet, but not when fed a regular chow diet. By contrast, wild-type C57BL/6 mice typically do not develop accelerated atherosclerosis on either a high cholesterol or a regular chow diet.

In one recent study, the present inventors found that LDLR−/− C57BL/6 mice immunized subcutaneously with 10 or 100 μg of heat-killed Mycobacterium tuberculosis and maintained on a normal chow diet for three months developed significantly larger fatty streaks than negative control mice immunized with bovine serum albumin (Afek A et al. J Autoimmun 2000; 14:115-121). These and other animal models can be used to select β₂GPI-derived peptides that are useful in accordance with the methods of the invention.

A therapeutically effective amount means an amount of active ingredients effective to induce an immune response thus preventing, alleviating or ameliorating symptoms of a disorder (e.g., atherosclerosis).

Ascertaining the optimum regimen for administering the active ingredient(s) is determined in light of the information disclosed herein and well known information concerning administration of mucosally active antigens, and autoantigens. Routine variation of dosages, combinations, and duration of treatment is performed under circumstances wherein the severity of atheromatous development can be measured. Useful dosage and administration parameters are those that result in reduction in inflammatory reaction, including a decrease in number of autoreactive T-cells, or in the occurrence or severity of at least one clinical or histological symptom of the disease.

In further preferred embodiments of the present invention, cytokine and non-cytokine synergists can be conjoined in the treatment to enhance the effectiveness of mucosal tolerization with plaque associated molecules. Oral and parenteral use of other cytokine synergists (Type I interferons) has been described in PCT/US95/04120, filed Apr. 7, 1995. Administration of Th2 enhancing cytokines is described in PCT application no. PCT/US95/04512, filed Apr. 7, 1995. For example, IL-4 and IL-10 can be administered in the manner described in PCT/US95/04512.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingi, et al., 1975, in “The Pharmacological Basis of Therapeutics”, Ch. 1 p. 1).

Dosage amount and interval may be adjusted individually to provide mucosal levels of the active ingredient that are sufficient to induce tolerance. The “tolerizing dosage” will vary for each preparation, but can be estimated from in vitro data. Dosages necessary to achieve tolerizing dosage will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise an inhaler. The pack or inhaler may be accompanied by instructions for administration. The pack or inhaler may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Thus, according to another aspect of the present invention there is provided an article of manufacture, packaged and identified for use in modulating an immune response to an atheroma plaque antigen in a subject in need thereof. The article of manufacture includes a packaging material and a mucosal tolerance-inducing amount of at least one beta₂-glycoprotein-1 (β₂GPI)-derived peptide, preferably a combination of at least two beta₂-glycoprotein-1 (β₂GPI)-derived peptides, the packaging material including a label or package insert indicating that the mucosal tolerance-inducing amount of the active ingredient is for modulating an immune response to an atheroma plaque antigen in the subject via mucosal administration.

According to yet a further aspect of the present invention there is provided an article of manufacture, packaged and identified for use in the prevention and/or treatment of a vascular condition in a subject in need thereof. The article of manufacture includes a packaging material and a mucosal tolerance-inducing amount ofat least one beta₂-glycoprotein-1 (β₂GPI)-derived peptide, preferably a combination of at least two beta₂-glycoprotein-1 (β₂GPI)-derived peptides, the packaging material including a label or package insert indicating that the mucosal tolerance-inducing amount of the active ingredient is for prevention and/or treatment of a vascular condition in the subject via mucosal administration.

It will be appreciated that the β₂GPI-derived peptides and chimeric peptides of the present invention can be administered in a non mucosal manner, i.e. as an active ingredient of a pharmaceutical composition. As is further detailed hereinunder and exemplified in the Examples section that follows, the beta₂-glycoprotein-1 (β₂GPI)-derived peptides of the present invention have a variety of therapeutic effects. In the Examples section there are provided numerous assays with which one of ordinary skills in the art can test a specific peptide designed in accordance with the teachings of the present invention for a specific therapeutic effect. Any of the peptides or combinations thereof described herein can be administered per se or be formulated into a pharmaceutical composition which can be used for treating or preventing a disease. Such a composition includes as an active ingredient any of the peptides described herein and a pharmaceutically acceptable carrier.

As used herein a “pharmaceutical composition” refers to a preparation of one or more of the peptides described herein, with other chemical components such as pharmaceutically suitable carriers and excipients. The purpose of a pharmaceutical composition is to facilitate administration of a compound to an organism.

Hereinafter, the term “pharmaceutically acceptable carrier” refers to a carrier or a diluent that does not cause significant irritation to an organism and does not abrogate the biological activity and properties of the administered compound. Examples, without limitations, of carriers are: propylene glycol, saline, emulsions and mixtures of organic solvents with water. Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition.

Suitable routes of administration may, for example, include oral, rectal, transmucosal, transdermal, intestinal or parenteral delivery, including intramuscular, subcutaneous and intramedullary injections as well as intrathecal, direct intraventricular, intravenous, intraperitoneal, intranasal, or intraocular injections.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more pharmaceutically acceptable carriers comprising excipients and auxiliaries, which facilitate processing of the active peptides into preparations which, can be used pharmaceutically. Proper formulation is dependent upon the route of administration chosen.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include biochemical and immunological techniques. Such techniques are thoroughly explained in the literature. See, for example, “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; and “Methods in Enzymology” Vol. 1-317, Academic Press; Marshak et al., all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Methods

Animals: Apo-E deficient mice used in these experiments are from the atherosclerosis prone strain C57BL/6]-Apoe^{tmlunc}. Mice homozygous for the Apoe^{tmlunc} mutations show a marked increase in total plasma cholesterol levels which is unaffected by age or sex. Fatty streaks in the proximal aorta are found at 3 months of age. The lesions increase with age and progress to lesions with less lipid but more elongated cells, typical of a more advanced stage of pre-atherosclerotic lesion.

LDL-RD mice (hybrids of a cross between the C57BL/6J and 129Sv strains) were previously created with homologous recombination as described by Ishibashi et al. (J Clin Invest 1993; 92:883-93). The LDL-RD mice have less highly elevated plasma cholesterol levels than the Apo-E deficient mice, but are also susceptible to atherosclerosis. All mice that were used in the experiment were females of age 6 weeks. The LDL-RD mice were obtained from Jackson Laboratories and bred at the local animal facility, as described for the Apo-E deficient mice.

Strain Development: The Apoe^{tmlunc} mutant strain was developed in the laboratory of Dr. Nobuyo Maeda at University of North Carolina at Chapel Hill. The 129-derived E14Tg2a ES cell line was used. The plasmid used is designated as pNMC109 and the founder line is T-89. The C57BL/6J strain was produced by backcrossing the Apoe^{tmlunc} mutation 10 times to C57BL/6J mice (11,12). The mice were maintained at the Sheba Hospital Animal Facility (Tel-Hashomer, Israel) on a 12-hour light/dark cycle, at 22-24° C. and fed a normal fat diet of laboratory chow (Purina Rodent Laboratory Chow No. 5001) containing 0.027% cholesterol, approximately 4.5% total fat, and water, ad libitum. “Western diet” (TD 96125, Harlan Teklad, 42% calories from fat, 43% from carbohydrates and 15% from protein) describes a standardized, high fat atherogenic diet.

Nasal Tolerance: Nasal tolerance was induced by intranasal administration of oxidized LDL, β2GPI, β2GPI-derived peptides or HSP65, in a total volume of 10 μl PBS. Intranasal administration was performed on mildly sedated mice (12-16 weeks old), each mouse receiving 3 doses of antigen per dose, in the indicated concentrations, every other day. Atherogenesis was induced by 5 weeks of a Western diet initiated on the day following the last intranasal administration, or assessed (in Apo E KO mice) after 8 weeks on a chow diet. Controls received equal amounts of BSA and/or PBS, as indicated, in an identical regimen. Plasma was obtained for assessment of cholesterol and triglyceride levels from all mice, and the mice were sacrificed for evaluation of atherosclerosis, as described hereinbelow, after 5 weeks Western diet, or (in Apo E KO mice) after 8 weeks on a chow diet.

Oral Tolerance: For comparison, oral tolerance to plaque-associated molecules was induced by feeding 3 doses of antigen every other day (for a detailed account of induction of oral tolerance, see U.S. patent application Ser. No. 09/806,400 to Shoenfeld et al filed Sep. 30, 1999), in a similar regimen to the nasal tolerance. LDL-RD mice were fed by a nasogastric tube, five doses (every other day) of human or bovine β₂GPI in PBS in two different concentrations (500 and 50 μg/dose). Control mice were either fed an irrelevant antigen (BSA; 50 μg) or not fed any antigen.

Oral tolerance to β2GPI-derived peptides, alone or in combination was induced by feeding 100 μg of the peptides (prepared in PBS, 0.2 ml), as detailed hereinabove, every other day for a total of 5 doses of 100 μg. One day following the last feeding, all mice were switched from chow-diet to an atherogenic “Western” diet and sacrificed five weeks later.

Antigen Preparation

β2GPI: Human and bovine β2GPI was purified from the serum of a healthy adult as described (Gharavi, et al, J Clin Invest 1992; 92:1105-09; George et al Circulation 1998; 15:1108-15). To ensure purity, pooled plasma was subsequently chromatographed on a heparin-SEPHAROSE column, on a DEAE-cellulose column, and on an anti-β2GPI affinity column. To remove any contamination by IgGs, the β2GPI-rich fraction was further passed through a protein A-SEPHAROSE column.

Oxidized LDL: Human LDL (density=1.019-1.063 g/l) was prepared from plasma of fasting individuals by preparative ultracentrifugation (50,000 rpm/min, 22 min), washing, dialysis against 150 mM EDTA, pH 7.4, filtration (0.22 μm pore size) to remove aggregation, and storage under nitrogen. LDL oxidation was performed by incubation of dialyzed, EDTA free LDL with copper sulfate (10 μM) for 16-24 hours at 37° C. Lipoprotein oxidation was confirmed by analysis of thiobarbituric acid-reactive substances (TBARS) which measures malondialdehyde (MDA) equivalents.

HSP65: Recombinant mycobacterial HSP-65, prepared as described (Prohaszka Z et al, Int Immunol 1999; 11:1363-70) was kindly provided by Dr. M. Singh, Braunschweig, Germany.

β2GPI Peptides: Synthetic peptides derived from human β2GPI (SEQ ID NO: 10) were synthesized according to standard peptide synthesis protocol, essentially as described by Ito et al (Hum Immunol 2000; 61:366-377), representing overlapping portions of approximately 20 amino acids each of the human β₂GPI polypeptide sequence (SEQ ID NO: 10). Peptides were designated S-1 (SEQ ID NO: 11), S-2 (SEQ ID NO: 12), S-3 (SEQ ID NO: 13) and S-4 (SEQ ID NO: 14), corresponding to peptides p64-83, p154-174, p244-264 and p271-291, respectively, of Ito et al. (Hum Immunol 2000).

Serial twelve-mer oligomeric β2GPI-derived peptides based on the amino acid sequence of peptide S-4 (SEQ ID NO: 14) were synthesized as mentioned hereinabove for peptides S-4-1 to S-4-10. The serial twelve-mer peptides are designated S-4-1, S-4-2, S-4-3 . . . S-4-10, (SEQ ID NO:15-24, respectively) and are described in detail in Table XXX hereinbelow.

| | | Aa coordinates | |
|---|---|---|---|
| | | (according to | SEQ ID |
| Peptide | Sequence | SEQ ID NO: 10) | NO: |
| S-1 | VCPFAGILENGAVRYTTFEY | 83-92 | 11 |
| S-2 | ECLPQHAMFGNDTITCTTHGN | 173-193 | 12 |
| S-3 | SCKVPVKKATVVYQGERVKIQ | 163-283 | 13 |
| S-4 | MLHGDKVSFFCKNKEKKCSYT | 290-310 | 14 |
| S-4-1 | MLHGDKVSFFCK | 290-301 | 15 |
| S-4-2 | LHGDKVSFFCKN | 291-302 | 16 |
| S-4-3 | HGDKVSFFCKNK | 292-303 | 17 |
| 5-4-4 | GDKVSFFCKNKE | 293-304 | 18 |
| S-4-5 | DKVSFFCKNKEK | 294-305 | 19 |
| S-4-6 | KVSFFCKNKEKK | 295-306 | 20 |
| S-4-7 | VSFFCKNKEKKC | 296-307 | 21 |
| S-4-8 | SFFCKNKEKKCS | 297-308 | 22 |
| S-4-9 | FFCKNKEKKCSY | 298-309 | 23 |
| S-4-10 | FCKNKEKKCSYT | 299-310 | 24 |

Immunization: Subcutaneous immunization with human β2GPI: Human β2GPI was prepared from human plasma pool as described above. For immunization, human β2GPI was dissolved in PBS and mixed with equal volumes of Freund's incomplete adjuvant. Immunizations were performed by single subcutaneous injection of 10 μg antigen/mouse in 0.1 ml volume. Three days following the last mucosal administration of plaque associated molecules the mice received one immunization, and were sacrificed 10 days post immunization.

Intraperitoneal immunization with human β2GPI-derived peptides: human β2GPI-derived peptides were dissolved in PBS and mixed with equal volumes of Freund's incomplete adjuvant. Immunizations were performed by 4 intraperitoneal injections of 20 μg antigen/mouse in 0.1 ml volume, administered once every other week. One week following the last immunization, the diet was switched from chow to atherogenic “Western” diet for five weeks, and then the mice were sacrificed.

Cholesterol Level Determination: At the completion of the experiment, 1-1.5 ml of blood was obtained by cardiac puncture, 1000 U/ml heparin was added to each sample and total plasma cholesterol levels were determined using an automated enzymatic technique (Kit No. 816302, Boehringer, Mannheim, Germany).

FPLC Analysis: Fast Protein Liquid Chromatography analysis of cholesterol and lipid content of lipoproteins was performed using Superose 6 HR 10/30 column (Amersham Pharmacia Biotech, Inc, Peapack, N.J.) on a FPLC system (Pharmacia LKB. FRAC-200, Pharmacia, Peapack, N.J.). A minimum sample volume of 300 μl (blood pooled from 3 mice was diluted 1:2 and filtered before loading) was required in the sampling vial for the automatic sampler to completely fill the 200 μl sample loop. Fractions 10-40 were collected, each fraction contained 0.5 ml. A 250 μl sample from each fraction was mixed with freshly prepared cholesterol reagent or triglyceride reagent respectively, incubated for 5 minutes at 37° C. and assayed spectrophotometrically at 500 nm.

Assessment of Atherosclerosis: Quantification of atherosclerotic fatty streak lesions was done by calculating the lesion size in the aortic sinus as previously described (16) and by calculating the lesion size in the aorta. Briefly, after perfusion with saline Tris EDTA, the heart and the aorta were removed from the animals and the peripheral fat cleaned carefully. The upper section of the heart was embedded in OCT medium (10.24% w/w polyvinyl alcohol; 4.26% w/w polyethylene glycol; 85.50% w/w nonreactive ingredients) and frozen. Every other section (10 μm thick) throughout the aortic sinus (400 μm) was taken for analysis. The distal portion of the aortic sinus was recognized by the three valve cusps that are the junctions of the aorta to the heart. Sections were evaluated for fatty streak lesions after processing and staining with oil-red O, according to Paigen et al (Atherosclerosis, 1987; 68:231-40). Lesion areas per section were scored on a grid (17) by an observer counting unidentified, numbered specimens. The aorta was dissected from the heart and surrounding adventitious tissue was removed. Fixation of the aorta and Sudan staining of the vessels were performed as previously described (21).

Immunohistochemistry of atherosclerotic lesions: Immunohistochemical staining for CD3, macrophages and β₂GPI content were done on aortic sinus 5-μm-thick frozen sections. Primary antibodies used for probing were rat anti-mouse CD3, rat anti-mouse Mac-1 and a polyclonal rat anti-mouse β2GPI antibodies (George et al Circulation 2000; 102:1822-7). Slides were developed with the three amino-9-ethylcarbonasole (AEC) substrate. Sections were counterstained with hematoxylin. Spleen sections were used as a positive control. Staining in the absence of 1st or 2nd antibody was used as a negative control. β2GPI presence were evaluated by its occupancy of plaque area by computerized morphometry as described previously for VCAM-1 (George et al, Circ Res. 2000; 86:1203-10).

Proliferation assays: Mice were exposed to the tested antigen as described for assessment of atherosclerosis, and then immunized (one to three days following the last exposure) subcutaneously with 10 μg β2GPI in 0.1 ml PBS, prepared from purified human β2GPI as described above.

Proliferation was assayed ten days after immunization with the β2GPI as follows: Draining inguinal lymph nodes were prepared by mashing the tissues on 100 mesh screens. Red blood cells were lysed with cold sterile double distilled water (6 ml) for 30 seconds and 2 ml of NaCl 3.5% was added. Incomplete medium was added (10 ml), cells were centrifuged for 7 min at 1,700 rpm, resuspended in RPMI medium and counted in a haemocytometer at 1:20 dilution (10 μl cells+190 μl Trypan Blue). Proliferation was measured by the incorporation of [³H] Thymidine into DNA in triplicate samples of 100 μl of the packed cells (1×10⁶ cells/ml) in a 96 well microtiter plate. Triplicate samples of β2GPI (10 μg/ml, 100 μl/well) or BSA were added, cells incubated for 72 hours (37° C., 5% CO₂ and ˜98% humidity) and 10 μl ³[H] Thymidine (0.5 μCi/well) was added. After an additional 12-24 hours of incubation the cells were harvested and transferred to glass fiber filters using a cell harvester (Brandel) and counted using β-counter (Lumitron). Proliferation was measured by the incorporation of [³H] thymidine into DNA during the final 12 h of incubation. The results are expressed as the stimulation index (S.I.): the ratio of the mean radioactivity (cpm) of the antigen to the mean background (cpm) obtained in the absence of the antigen. Standard deviation was always <10% of the mean cpm.

For assessment of mucosal tolerance with β₂GPI or β₂GPI-derived peptides on reactivity to oxidized LDL, mice (n=4) were exposed to tolerizing doses of β2GPI, β2GPI-derived peptides or BSA in three or five doses, as described hereinabove. One day following the last dose, all mice were immunized with human ox-LDL or BSA (10 μg/ml) emulsified in Freund's incomplete adjuvant, and draining lymph nodes collected 10 days later. Proliferation in response to oxLDL stimulus was assessed substantially as above: 3×10⁵ lymph node cells/well were incubated in triplicates for 72 h in 0.2 ml of culture medium in microtiter wells in the presence of 2.5 μg/ml 0xLDL. Proliferation was measured by the incorporation of [³H] thymidine into DNA during the final 12 h of incubation. The results were computed as stimulation index: the ratio of the mean cpm of the antigen to the mean background cpm obtained in the absence of the antigen.

IFN-γ, IL-4, IL-10 and TGF-β secretion in tolerized lymph nodes: Conditioned medium was obtained from the lymph node proliferation experiments following 48 h of culture in the presence of β2GPI. IFN-g, IL-4, IL-10 and TGF-h concentrations were determined by ELISA kits according to the manufacturer's suggestions (R&D Systems Inc., Minneapolis, Minn.).

RT-PCR analysis of cytokine expression: 7-9 week old male ApoE-KO mice were tolerized by oral administration of β2GPI in 5 feedings, every other day, of human β2GPI (100 μg/mouse) or PBS, as control, by gavage, as detailed hereinabove. Three days following the oral administration of β2GPI, the mice were sacrificed, aortas collected and processed for RT-PCR analysis of the expression of anti-inflammatory Th2 type cytokine IL-10 and the proinflammatory Th1-type cytokine IFN-γ. The RT-PCR analysis was performed according to the protocol as described in detail by Colle et al (Journal of Immunol Methods 1997; 175-184). Briefly, RNA was extracted from the aortal tissue and reverse transcribed according to well-known, standard protocols, and the transcription products subjected to PCR amplification using the following primers: IL-10-forward primer 5′ CTGGACAACATACTGCTAACCGAC 3′ (SEQ ID NO: 1), located at nucleotide positions 256-278 of IL-10 (GenBank Accession No. NM 010548 (SEQ ID NO: 2)) and reverse primer 5′ATTCATTCAYGGCCTTGTAGACACC 3′ (SEQ ID NO: 3), located at nucleotide positions 532-556 of IL-10 (GenBank Accession No. NM 010548 (SEQ ID NO: 2)); IFN-γ-forward primer 5′ CTTCTTCAGCAACAGCAAGGCGAAAA 3′ (SEQ ID NO: 4), located at nucleotide positions 372-397 of IFN-γ (GenBank Accession No. NM 008337 (SEQ ID NO: 5)), reverse primer 5′ CCCCCAGATACAACCCCGCAATCA 3′ (SEQ ID NO: 6), located at nucleotide positions 804-827 of IFN-γ (GenBank Accession No. NM 008337 (SEQ ID NO: 5)); and β-actin-forward primer 5′ GGACTCCTATGTGGGTGACGAGG 3′ (SEQ ID NO: 7), located at nucleotide positions 230-252 of β-actin (GenBank Accession No. NM 007393 (SEQ ID NO: 8)), and reverse primer 5′ GGGAGAGCATAGCCCTCGTAGAT 3′ (SEQ ID NO: 9), located at nucleotide positions 573-579 of β-actin (GenBank Accession No. NM 007393 (SEQ ID NO: 8)). The resultant amplified IL-10, IFN-γ and β-actin transcripts were separated by electrophoresis on an agarose gel, and visualized by ehtidium bromide staining.

Detection of anti-b2GPI antibodies and antibody isotypes: β2GPI (10 μg/ml) was coated onto flat bottom 96-well ELISA plates (Nalge-Nunc, Int. Rochester, N.Y.) by overnight incubation and the assay was performed as previously described (George et al, Circulation, 1998; 15:1108-15) IgG, IgA and IgM isotypes in the sera of β2GPI tolerant and non-tolerant mice were determined by an ELISA kit (Southern Biotechnology Associates, Birmingham, Ala., USA) according to the manufacture's instructions.

Statistical Analysis: A one-way ANOVA test was used to compare independent values. p<0.05 was accepted as statistically significant.

### Example 1

### Inhibition of Atherogenesis in Genetically Predisposed (LDL Receptor-Deficient) Mice by Induction of Nasal Tolerance with Low Doses of the Plaque Associated Molecules Oxidized LDL, Human β2GPI and HSP 65

The present inventors here demonstrate that mucosal administration, via nasal exposure, to low doses of the plaque associated molecules oxidized LDL, β₂GPI and HSP 65 provides induction of immune tolerance to the antigens, and significant inhibition of atherogenesis. Thus, nasal exposure to purified, oxidized human LDL, human β₂GPI and recombinant mycobacterial HSP 65 were compared for their effectiveness in suppressing atherogenesis in LDL-RD mice. 63 male 9-13 week old LDL RD mice were divided into 5 groups. In group A (HSP-65)(n=12) nasal tolerance was induced as described in Materials and Methods by administration of recombinant mycobacterial HSP 65 suspended in PBS (10 μg/mouse/10 μl) for 5 days every other day. In group B (H-oxLDL)(n=14) nasal tolerance was induced as described in Materials and Methods by administration of 10 μg/mouse/10 μl oxidized purified human LDL, suspended in PBS, every other day for 5 days. Mice in group C (B₂GPI)(n=13) received 10 μg/mouse/10 μl human β2GPI per mouse, administered intranasally as described in Materials and Methods, every other day for 5 days. Mice in group D (BSA)(n=12) received 10 μg/mouse/10 μl bovine serum albumin (BSA) per mouse, administered intranasally as described in Materials and Methods, every other day for 5 days. Mice in group E (PBS)(n=12) received 10 μl PBS per mouse, administered intranasally. Mice were bled prior to feeding (Time 0) and at the conclusion of the experiment (End) for determination of lipid profile. Atherosclerosis was assessed in heart and aorta as described above, 8 weeks after the last feeding. Mice were weighed every 2 weeks during the experiment. All mice were fed water ad libitum and a normal chow-diet containing 4.5% fat by weight (0.02% cholesterol), up to the final antigen exposure, and then a “Western” diet until sacrifice.

TABLE 1
Inhibition of atherogenesis in LDL receptor-deficient mice by intranasal
administration of exceedingly low doses of plaque associated molecules

| Time | | HSP-65 | H-oxLDL | Hβ₂GPI | BSA | PBS | Statistics |
|---|---|---|---|---|---|---|---|
| Day 0 | Weight (g) | 22.6 ± 0.8 | 22.3 ± 0.5 | 22.3 ± 0.7 | 21.8 ± 0.7 | 21.7 ± 0.5 | p = 0.833* |
| | Cholesterol | 237 ± 13 | 230 ± 10 | 230 ± 14 | 236 ± 19 | 227 ± 14 | P = 0.986* |
| | (mg/dL) | | | | | | |
| | Triglyceride | 150 ± 19 | 178 ± 17 | 162 ± 18 | 185 ± 22 | 160 ± 15 | P = 0.664* |
| | (mg/dL) | | | | | | |
| END | Weight (g) | 26.8 ± 0.9 | 28.2 ± 1.0 | 29.2 ± 1.5 | 25.5 ± 1.0 | 26.3 ± 1.3 | P = 0.157* |
| | Cholesterol | 1181 ± 114 | 1611 ± 119 | 1601 ± 125 | 1470 ± 183 | 1606 ± 181 | P = 0.197* |
| | (mg/dL) | | | | | | |
| | Triglyceride | 288 | 275 | 380 | 315 | 403 | P = 0.416** |
| | (mg/dL) | | | | | | |
| | Aortic Sinus | 44375 ± 5437 | 43393 ± 4107 | 46250 ± 4486 | 120500 ± 8746 | 128182 ± 9102 | P < 0.001* |
| | Lesion (μm²) | | | | | | |

*One way ANOVA (Mean ± S.E)
**Kruskal-Wallis One Way Analysis of Variance on Ranks (Median)

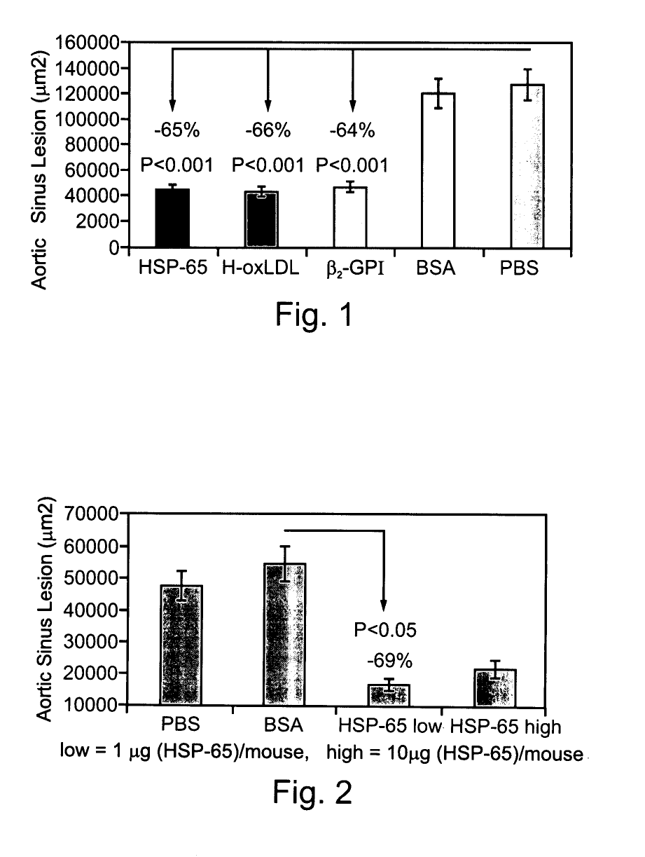

As can be seen from FIG. 1, the results depicted in Table 1 demonstrate the strikingly effective inhibition of atherogenesis measured in the tissues of mice receiving mucosal (nasal) exposure to low doses (10 μg/mouse) of the plaque associated molecules, compared to control mice exposed to sham antigen (BSA) or PBS. Furthermore, nasal tolerance is specific in its mode of protection: clearly, induction of nasal tolerance has no significant effect on other general parameters measured, such as weight gain, triglyceride or cholesterol blood levels. Thus, the antigenic plaque associated molecules oxidized LDL, β₂GPI and HSP 65 are highly potent inducers of mucosal tolerance, when administered nasally, with surprisingly low doses (10 μg/mouse) and brief exposure (3 days) of significant (greater than 65%) and consistent protection from atherogenesis in these genetically susceptible LDL receptor-deficient mice.

### Example 2

### Superior Inhibition of Atherogenesis in Genetically Predisposed (LDL-RD) Mice by Induction of Nasal Tolerance with HSP 65

The present inventors here demonstrate, that mucosal administration, by nasal exposure to exceedingly low doses of the plaque associated molecule HSP 65 provides superior induction of tolerance to the antigen, and inhibition of atherogenesis. Thus, nasal exposure to a low dose and an exceedingly low dose of recombinant human HSP 65 were compared for their effectiveness in suppressing atherogenesis in LDL-RD mice. 58 male 12-16 week old LDL-RD mice were divided into 4 groups. In group A (HSP-65 high)(n=14) nasal tolerance was induced as described in Materials and Methods by intranasal administration of 10 μg/mouse/10 μl recombinant human HSP 65 suspended in PBS for 5 days every other day. In group B (HSP-65 low)(n=16) nasal tolerance was induced as described in Materials and Methods by administration of 1 μg/mouse/10 μl recombinant human HSP 65 suspended in PBS every other day for 5 days. Mice in group C (BSA)(n=14) received 1 μg/mouse/10 μl BSA per mouse, administered intranasally, every other day for 5 days. Mice in group D (PBS)(n=14) received 10 μl PBS per mouse, administered intranasally. Mice were bled prior to feeding (Time 0) and at the conclusion of the experiment (End) for determination of lipid profile. Atherosclerosis was assessed in heart and aorta as described above, 8 weeks after the last feeding. Mice were weighed every 2 weeks during the experiment. All mice were fed water ad libitum and a normal chow-diet containing 4.5% fat by weight (0.02% cholesterol), up to the final antigen exposure, and then a “Western” diet until sacrifice.

TABLE 2
Superior inhibition of atherogenesis in LDL-receptor-deficient
mice by intranasal administration of human HSP 65

| | | HSP65 | HSP65 | BSA | | |
|---|---|---|---|---|---|---|
| | | 10 μg/ | 1 μg/ | 100 μg/ | | |
| | | Mouse | Mouse | Mouse | PBS | |
| | | N = 12 | N = 16 | N = 11 | N = 10 | Statistics* |
| End | Weight (g) | 28.4 ± 1.0 | 26.9 ± 0.9 | 27.7 ± 0.5 | 28.7 ± 0.7 | P = 0.363 |
| | Cholesterol | 1073 ± 65 | 1010 ± 64 | 1009 ± 74 | 1015 ± 85 | P = 0.897 |
| | (mg/dL) | | | | | |
| | Triglyceride | 348 ± 32 | 315 ± 46 | 316 ± 32 | 390 ± 44 | P = 0.564 |
| | (mg/dL) | | | | | |
| | Aortic Sinus | 22292 ± 2691 | 17109 ± 2053 | 54432 ± 8201 | 47750 ± 5779 | P < 0.05 |
| | Lesion μm² | | | | | Between |
| | | | | | | HSP-65 and |
| | | | | | | PBS or BSA |

*One way ANOVA (Mean ± S.E)

As can be seen from FIG. 2, the results depicted in Table 2 demonstrate the superior effectiveness of inhibition of atherogenesis measured in the tissues of mice receiving nasal exposure to exceedingly low doses (1 μg/mouse) of HSP 65, compared to control mice exposed to sham antigen (BSA) or PBS. Furthermore, nasal tolerance is specific in its mode of protection: clearly, induction of nasal tolerance, has no significant effect on other general parameters measured, such as weight gain, triglyceride or cholesterol blood levels. Thus, the antigenic plaque associated molecule HSP 65 is an extremely potent inducer of nasal tolerance, with even exceedingly low doses conferring significant (approximately 70%) protection from atherogenesis in genetically susceptible LDL-RD mice, greatly superior to the protection achieved by induction of oral tolerance (30%, see U.S. patent application Ser. No. 09/806,400 to Shoenfeld et al filed Sep. 30, 1999, the contents of which are incorporated by reference as if fully set forth herein).

### Example 3

### Superior Suppression of Specific Anti-β2GPI Immune Reactivity in Genetically Predisposed (LDL-RD) Mice by Mucosal Administration of Human β₂GPI

Tolerance induced by mucosal exposure to plaque-associated molecules may be mediated by suppression of specific immune responses to antigenic portions (epitopes) of these plaque associated molecules. Lymphocyte proliferation in response to mucosal (nasal and oral) exposure to human β2GPI was measured in apoE-deficient mice. 9 male, 5 week old LDL-RD mice were divided into 3 groups. In group A (n=3) oral tolerance was induced with 100 μg/mouse β2GPI suspended in 0.1 ml PBS, administered by gavage, as described above, every other day for 5 days. In group B (n=3) nasal tolerance was induced with 10 μg/mouse β2GPI suspended in 10 μl PBS, administered intranasally as described above, every other day for 5 days. The mice in group C (n=3) received oral administration of 200 μl PBS every other day for 5 days. Immune reactivity was stimulated in all mice by immunization with human β2GPI as described above in the Materials and Methods section, one day after the last feeding. Ten days after the immunization lymph nodes were collected for assay of proliferation (as expressed in the Stimulation Index SI). All mice were fed normal chow-diet containing 4.5% fat by weight (0.02% cholesterol) and water ad libitum.

TABLE 3
Intranasal pretreatment with purified human β2 GPI suppresses
immune response to Human β₂GPI in LDL receptor-deficient mice

| | | H-β2-GPI | |
|---|---|---|---|
| | PBS | (Oral) | H-β2-GPI (Nasal) |
| S.I (Stimulation Index) | 7.0 ± 0.2 | 4.4 ± 0.5 | 2.1 ± 0.5 |

As can be seen from FIG. 3, the results depicted in Table 3 demonstrate significant suppression of immune reactivity to human β2GPI antigen, measured by inhibition of proliferation in the lymph nodes of LDL RD mice. Lymphocytes from mice receiving intranasal exposure to low atherogenesis-inhibiting doses (10 μg/mouse) of human β2GPI showed an exceedingly reduced stimulation index following immunization with β2GPI, as compared to orally exposed and control (PBS) mice. Since previous studies with induction of nasal tolerance have shown no significant effect on other parameters measured, such as weight gain, triglyceride or cholesterol blood levels, or immune competence (see abovementioned Examples), these results indicate a specific suppression of anti-β₂GPI immune reactivity. Thus, mucosal administration, by intranasal exposure, of the purified plaque associated molecule β₂GPI is a superior method of attenuating the cellular immune response to immunogenic and atherogenic plaque associated molecules in these genetically susceptible apoE-deficient mice.

### Example 4

### Mucosal Administration of β₂GPI Effectively Suppresses Atherogenesis in LDL-Receptor Deficient Mice

LDL-receptor deficient (LDL-RD; LDLR −/−) mice created on a C57BL/6 background develop accelerated atherosclerosis when fed a high cholesterol diet, but not when fed a regular chow diet. In order to determine whether mucosal administration of the plaque antigen β2GPI could suppress atherogenic processes, LDL-RD mice were fed low, oral tolerance inducing doses of human and bovine β2GPI, and the assessed for alterations in response to diet.

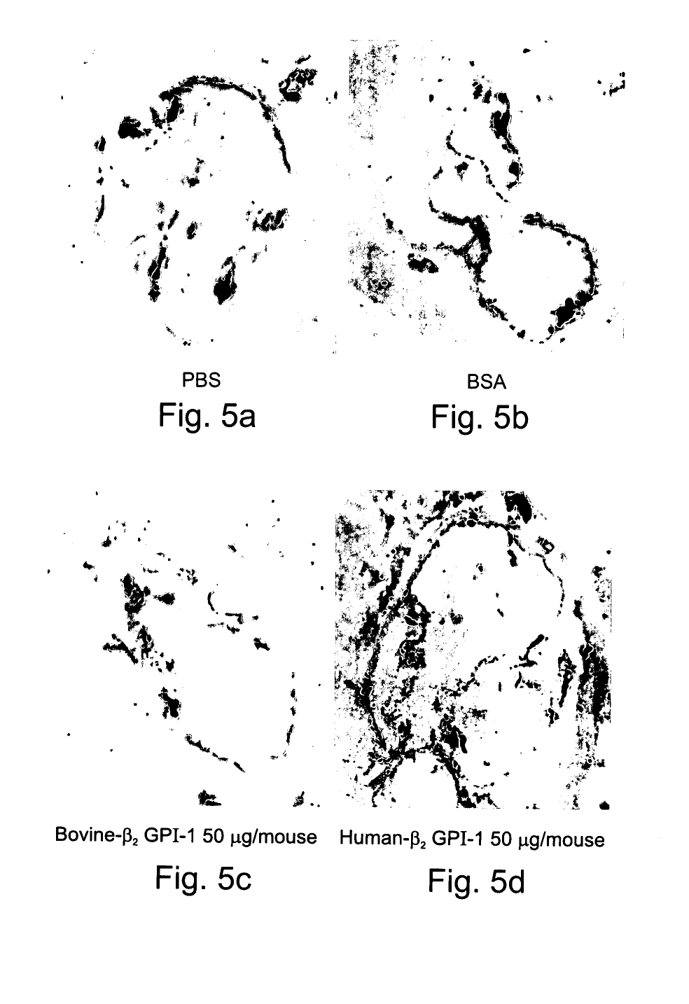

Oral administration of Bovine or Human β2GPI is specifically antiatherogenic in LDL-RD mice: Oral administration (via gavage, as described hereinabove) of human β₂GPI at 50 μg and at 500 μg/dose were similarly effective in suppressing atherosclerosis in the LDL-RD mice (45% and 44% reduction, respectively, as compared with BSA-fed controls). Oral administration of bovine β2GPI was also effective in reducing early atherosclerotic lesion size in both the low 500 μg and the exceedingly low 50 μg dosages (43% and 57% suppression, respectively (see FIGS. 4 and 5). Oral administration of BSA did not alter lesion progression in comparison with PBS (FIGS. 4 and 5).

In order to rule out non-specific, systemic effects of oral administration of β₂GPI, the lipid profiles of the treated and control mice were determined.

Oral administration of Bovine or Human β₂GPI does not significantly influence total cholesterol or triglyceride levels: Table 4 shows the results of oral administration of 500 μg or 50 μg of bovine (B-β₂) or human (H-β₂) β₂GPI, or BSA, to LDL-RD mice, as described above. No significant influence of β₂GPI administration, at either dose, on total cholesterol or triglycerides levels was observed, indicating that the antiatherogenic effects of oral β₂GPI administration do not result from alteration of the availability of plaque components.

TABLE 4
Lipid profile of mice tolerized orally with β2GPI

| | | H-β2 GPI | H-β2 GPI | B-β2 GPI | B-β2 GPI | BSA | |
|---|---|---|---|---|---|---|---|
| | | (500 μg) | (50 μg) | (500 μg) | (50 μg) | (500 μg) | PBS |
| Start | Weight (g) | 27.1 ± 0.7 | 26.4 ± 0.7 | 25.7 ± 0.7 | 26.4 ± 0.7 | 26.8 ± 0.7 | 26.5 ± 0.6 |
| | Cholesterol | 231 ± 16 | 227 ± 14 | 233 ± 15 | 231 ± 14 | 231 ± 17 | 232 ± 12 |
| | (mg/dL) | | | | | | |
| | Triglyceride | 182 ± 18 | 212 ± 26 | 178 ± 23 | 179 ± 20 | 188 ± 26 | 189 ± 23 |
| | (mg/dL) | | | | | | |
| End | Weight (g) | 31.4 ± 1.0 | 32.0 ± 0.6 | 29.8 ± 0.9 | 30.8 ± 0.8 | 30.5 ± 0.8 | 31.6 ± 0.6 |
| | Cholesterol | 1232 ± 92 | 1237 ± 71 | 1196 ± 75 | 1200 ± 76 | 1166 ± 91 | 1250 ± 90 |
| | (mg/dL) | | | | | | |
| | Triglyceride | 415 ± 62 | 342 ± 51 | 318 ± 55 | 313 ± 51 | 293 ± 45 | 424 ± 54 |
| | (mg/dL) | | | | | | |
| | Aortic Sinus | 27031 ± 3387 | 26563 ± 2370 | 20781 ± 2290 | 27422 ± 3007 | 48167 ± 3340 | 50667 ± 5703 |
| | Lesion μm² | | | | | | |

H-β2-human β2GPI, B-β2-bovine β2GPI.
Data displayed as mean ± S.E

In order to assess whether oral administration of β₂GPI affected endogenous β₂GPI, the content of plaque-expressed β₂GPI was measured in mice receiving oral β₂GPI or BSA administration. Oral administration of β₂GPI (500 μg/dose) did not have a significant influence on the content of plaque-expressed β₂GPI (mean percent occupancy of 15±5) in comparison with BSA feeding (occupancy of 19±7%)(data not shown). In order to assess whether oral administration of β₂GPI affected the inflammatory phenotype of the fatty streaks, CD3-positiveand macrophage positive cells were measured in mice receiving oral β₂GPI or BSA administration. No effect of oral β₂GPI on the number of CD3 positive cells (0-5 cells/plaque in all groups; data not shown) or macrophage (Mac-1 positive) content of the fatty streaks was observed.

Thus, suppression of atherosclerosis by oral administration of β2GPI was not the result of reduced availability of the autoantigen at the site of the lesion, nor of a change in the inflammatory immune-cell profile of the plaque lesions.

### Example 5

### Superior Inhibition of Atherogenesis in Genetically Predisposed (apoE-Deficient) Mice by Induction of Mucosal Tolerance with Mucosal Administration of β2GPI

Mucosal administration of Human β₂GPI specifically inhibits progression of advanced atherogenic processes in ApoE-KO mice: Adult ApoE-KO mice develop advanced atherosclerotic lesions when fed an atherogenic “Western diet”. In order to determine the protective effect of mucosal administration of β2GPI on development of atherosclerosis, adult ApoE-KO mice were treated with oral administration of human β2GPI. 20 week-old male ApoE-KO mice, having advanced atherosclerotic lesions were treated monthly with human β2GPI (50 μg/dose) or PBS (0.2 ml) in 5 oral administrations (by gavage, as described hereinabove) given every other day, for four months. Lesion area calculated from cryosections of the aortic sinus were compared between control untreated 20 week old mice, and mice following 16 weeks oral administration of β2GPI or PBS.

As shown in FIG. 6, 16 weeks after initiating treatment, atherosclerosis (aortic lesions) in the PBS treated controls had progressed 124% over initial lesions in the week old mice. Oral administration of 50 μg human β2GPI during the following 16 weeks inhibited the progression of atherosclerotic lesions (35% reduction) as compared to PBS control.

In order to rule out non-specific, systemic effects of oral administration of β2GPI, the lipid profiles of the treated and control mice were determined.

Oral administration of Human β₂GPI does not significantly influence metabolic profile of mice having advanced atherosclerosis: Table 5 shows the results of oral administration of 50 μg of human (H-β₂) β₂GPI, or PBS, to adult male Apo-E KO mice, as described above. No significant influence of β₂GPI administration, at either dose, on body weight, total cholesterol or triglycerides levels was observed, indicating that the inhibition of atherosclerotic progression in adult male Apo-E KO by oral β₂GPI administration does not result from alteration of the availability of plaque components or lipid metabolism.

TABLE 5
Lipid profile of mice tolerized orally with β2GPI

| | | UNTREATED | | B-β₂GPI |
|---|---|---|---|---|
| | | (week 16) | PBS | (50 μg/mouse) |
| Start | Weight (g) | 25.5 ± 0.7 | 265.5 ± 0.7 | 25.5 ± 0.4 |
| t = 0 | | | | |
| | Cholesterol | 361 ± 28 | 361 ± 20 | 360 ± 17 |
| | (mg/dL) | | | |
| | Triglyceride | 76 ± 9 | 76 ± 5 | 71 ± 17 |
| | (mg/dL) | | | |
| End | Weight (g) | NA | 28.1 ± 0.6 | 29.6 ± 0.8 |
| t = 16 wks | | | | |
| | Cholesterol | NA | 1494 ± 21 | 505 ± 42 |
| | (mg/dL) | | | |
| | Triglyceride | NA | 114 ± 7 | 109 ± 9 |
| | (mg/dL) | | | |
| | Aortic Sinus Lesion | 152321 ± 6106 | 345000 ± 18370 | 221042 ± 14472 |
| | μm² | | | |

H-β2—human β2GPI.
Data displayed as mean ± S.E

### Example 6

### Mucosal Administration of β₂GPI Specifically Suppresses the Immune Response to β2GPI and Other Plaque-Related Autoantigens in Ldl-Receptor Deficient Mice

Nicoletti et al (Mol. Med. 2000; 6, 283-90) have shown that tolerance to the antigens in oxidized LDL, brought about by neonatal administration of β₂GPI, led to clonal anergy/deletion of the oxLDL reactive cells and to consequent suppression of atherosclerosis. The effect of oral administration of β₂GPI on the character of the immune response to β2GPI and other plaque related autoantigens was assessed in LDL-RD mice.

Oral administration of β₂GPI inhibits the cellular immune response to plaque related antigens: In order to assess the role of specific induction of immune tolerance in the antiatherogenic effects of oral administration of β₂GPI to LDL-RD mice, the extent of lymph node proliferation in response to challenge with β2GPI was compared in mice receiving oral β₂GPI or BSA administration. FIGS. 7A and 7B show the differences in thymidine uptake, expressed as Stimulation Index, between lymph node cells from LDL-RD mice immunized with β₂GPI (FIG. 7A) or oxidized LDL (oxLDL, FIG. 7B), following oral administration of β₂GPI or BSA, and exposure of the cells to the sensitizing antigen.

Oral administration of β₂GPI effectively inhibits the cellular immune response to the plaque related antigens in sensitized mice. FIG. 7A shows the significant inhibition of lymph node cell proliferation stimulated by β₂GPI in the β₂GPI tolerized mice, even at the exceedingly low doses of β2GPI also found effective in suppressing atherosclerosis (see FIG. 7A, 2.0 μg and 0.4 μg/ml). FIG. 7B shows the effect of oral administration of β₂GPI on lymph node cell proliferation stimulated by oxidized LDL. The results clearly show that prior oral administration of β₂GPI (5 doses of 500 μg per mouse) suppressed the primary cellular response to stimulation with both β2GPI (>60% suppression upon stimulation with 2.0 μg/ml β₂GPI) (FIG. 7A) and, surprisingly, also to the plaque antigen oxLDL (74% suppression upon stimulation with 20 μg/ml oxLDL)(FIG. 7B). Oral administration of β₂GPI did not influence the primary cellular response to BSA in mice immunized with BSA, and oral administration of BSA in mice immunized with oxLDL had no effect on the proliferative response to the sensitizing antigen (oxLDL) (results not shown). Thus, oral administration of β₂GPI results in specific suppression of the primary cellular immune response to β₂GPI as well as to other plaque autoantigens.

Changes in the lymph node cell cytokine profile following oral administration of β2GPI: To investigate whether suppression of lymph node cell reactivity to β₂GPI was associated with a change in cytokine production, conditioned medium from lymph node cells collected from mice receiving oral β₂GPI or BSA administration, following immunization with β₂GPI (50 g/mouse), and incubated for 48 hours in the presence of β₂GPI, was collected, and assayed for cytokines.

IL-4 and IL-10: The levels of anti-inflammatory type Th2 cytokines IL-4 and IL-10 were measured. Levels of IL-4 in medium from cells of animals receiving oral β₂GPI were three times higher (p<0.01) than those from lymph node cells from control animals (FIG. 8). A similar pattern was evident with regard to IL-10. Namely, lymph node cells from animals receiving oral β₂GPI administration following immunization with β₂GPI secreted significantly more IL-10 (2.6 times higher; p<0.05) upon in-vitro priming with β₂GPI than did lymph node cells from BSA-treated controls (FIG. 8).

IFN-γ and TGF-β: Levels of the proinflammatory Th1-type cytokine IFN-γ and the anti-inflammatory mediator of mucosal tolerance TGF-β were measured. Oral administration of β2GPI did not induce significant changes in the levels of IFN-γ secreted by lymph node cells in response to stimulation with β₂GPI (mean value of 1802±588 pg/ml in the cells from β₂GPI-tolerized mice as compared with 1870±378 pg/ml in cells from controls). TGF-β levels in the conditioned medium of lymph node cells obtained from β2GPI-tolerized and non-tolerized control mice were below the detection threshold.

Oral administration of β₂GPI induces anti-inflammatory cytokines in-vivo: To determine whether the changes in cytokine profile observed in lymph node cells of β₂GPI-tolerized mice reflected significant modulation of the inflammatory response of the affected tissue in-vivo, the cytokine expression profile of aorta tissue from ApoE-KO mice following a regimen of mucosal administration of β₂GPI was determined by RT-PCT using primers specific to IL-10, IFN-γ, and β-actin as control.

Male ApoE-KO mice 7-9 weeks of age were treated by oral administration of human β2GPI (100 μg/mouse) or PBS, as control, by gavage, as detailed hereinabove, 5 times, on every other day. Three days following the oral administration of β2GPI, the mice were sacrificed, and aortas collected and processed for RT-PCR analysis of the expression of anti-inflammatory Th2 type cytokine IL-10 and the proinflammatory Th1-type cytokine IFN-γ. The results of SDS-PAGE separation of the PCR products (FIG. 9) clearly show induction of expression of the anti-inflammatory, anti-atherogenic cytokine IL-10, and inhibition of expression of the proinflammatory cytokine IFN-γ within the plaqued regions of the aortas, without any influence on tissue levels of the housekeeping β-actin gene transcripts.

These results show, for the first time, that mucosal administration of β2GPI to subjects genetically predisposed to atherosclerosis, suppresses immune reactivity to β2GPI, and causes a shift in the immune profile, enhancing expression and tissue levels of anti-inflammatory cytokines and suppressing pro-inflammatory cytokines expression, in the lymph organs and in the aortic tissue itself.

Oral administration of β₂GPI does not affect antibody levels: To explore whether Th2 cytokine dominance in the lymph nodes of β₂GPI-tolerized mice was associated with a skewed antibody isotype distribution, total antibody levels as well as the anti-β₂GPI IgM, IgG and IgA antibody levels and isotypes were measured in sera of β₂GPI-tolerized mice that were subsequently immunized with β₂GPI. Oral administration of β₂GPI did not alter IgM, IgA, or IgG total antibody levels nor was there a change in isotype distribution in comparison with non-tolerant mice (data not shown). None of the orally-administered antigens induced production of antiβ2GPI antibodies (data not shown).

Thus, these results indicate that oral administration of β₂GPI results in increased levels of the Th2 type cytokines IL-10 and IL-4 in response to stimulation with β₂GPI, but no difference in levels of IFN-γ or TGF-β in lymph node cells from tolerized mice. On the other hand, the effects of oral administration of β₂GPI on cytokine expression in aorta tissue in-vivo clearly indicate enhanced anti-inflammatory IL-10 and suppression of proinflammatory IFN-γ expression.

Taken together, the results brought hereinabove unexpectedly reveal that mucosal administration, via both oral and nasal presentation of the plaque-related antigen β₂GPI according to the methods of the present invention effectively inhibits both early and late stage atherogenic processes and, although no change in the inflammatory cell infiltration, macrophage content or antibody profile was noted, mucosal β₂GPI administration results in induction of the Th2 type cytokines and has a strong suppressive effect on reactivity of sensitized immune cells to stimulation by β₂GPI. Without wishing to be limited by a single hypothesis, it is feasible that increased levels of IL-10 results in the striking antiatherogenic effects (inhibition of the proinflammatory nuclear factor-B, inhibition of matrix metalloproteinases, reduction of tissue factor expression, and inhibition of apoptosis of macrophages and monocytes) unrelated to Th1 cytokine suppression.

Further, the results reveal, for the first time, that mucosal administration of β2GPI prior to immunization of the mice with oxLDL significantly inhibits the primary immune responses to oxLDL stimulation. Without wishing to be limited by a single hypothesis, this tolerizing effect on oxLDL responsiveness can be mediated through the “bystander effect”, involving regulatory cells secreting nonantigen-specific cytokines that suppress inflammation in the microenvironment where the mucosally administered antigen is localized such as has been demonstrated for colon-distinctive protein (Gotesman et al, J Pharma and Expanding Ther. 2001; 297-32), pre-transplant splenocytes antigens (Ilan et al, Blood 2000; 95:3613-19) and myelin basic protein (Becker et al. PNAS USA 1997; 94:10873-78).

### Example 7

### Mucosal Administration of β2GPI Peptides Effectively Suppresses Atherogenesis in LDL-Receptor Deficient Mice

LDL-receptor deficient (LDL-RD; LDLR −/−) mice created on a C57BL/6 background develop accelerated atherosclerosis when fed a high cholesterol diet, but not when fed a regular chow diet. In order to determine whether mucosal administration of the plaque antigen β₂GPI could suppress atherogenic processes, LDL-RD mice were fed low, oral tolerance inducing doses of human and bovine β₂GPI, and assessed for alterations in response to diet. The results presented in Examples 1-6 of the instant specification hereinabove have suprisingly uncovered that mucosal administration of β₂GPI can reduce early atherosclerotic lesions and reduce the progression of advanced atherosclerotic plaques, suppress cellular immune responses and suppress inflammatory response in LDL-receptor deficient (LDL-RD; LDLR −/−) mice. However, the effective portions of the plaque antigen actively participating in the mucosal tolerance induced by β₂GPI are as yet not known.

Ito et al. (Ito et al., Hum Immunol 2000, 61:366-377) have analyzed T cell responses to a β₂GPI derived peptide library in patients with anti-β₂GPI antibody-associated autoimmunity, and identified specific peptides recognized by β2 GPI-reactive CD4+ T cells of APS/SLE patients, capable of stimulating anti-inflammatory cytokine (Th0 and Th2) production. The effect of mucosal administration of synthetic β₂GPI peptides (termed β₂GPI S-1 (SEQ ID NO: 11), S2 (SEQ ID NO: 12), S-3 (SEQ ID NO: 13) and S-4 (SEQ ID NO: 14) on atherosclerosis and atherogenic processes was assessed in LDL-receptor deficient (LDL-RD; LDLR −/−) mice.

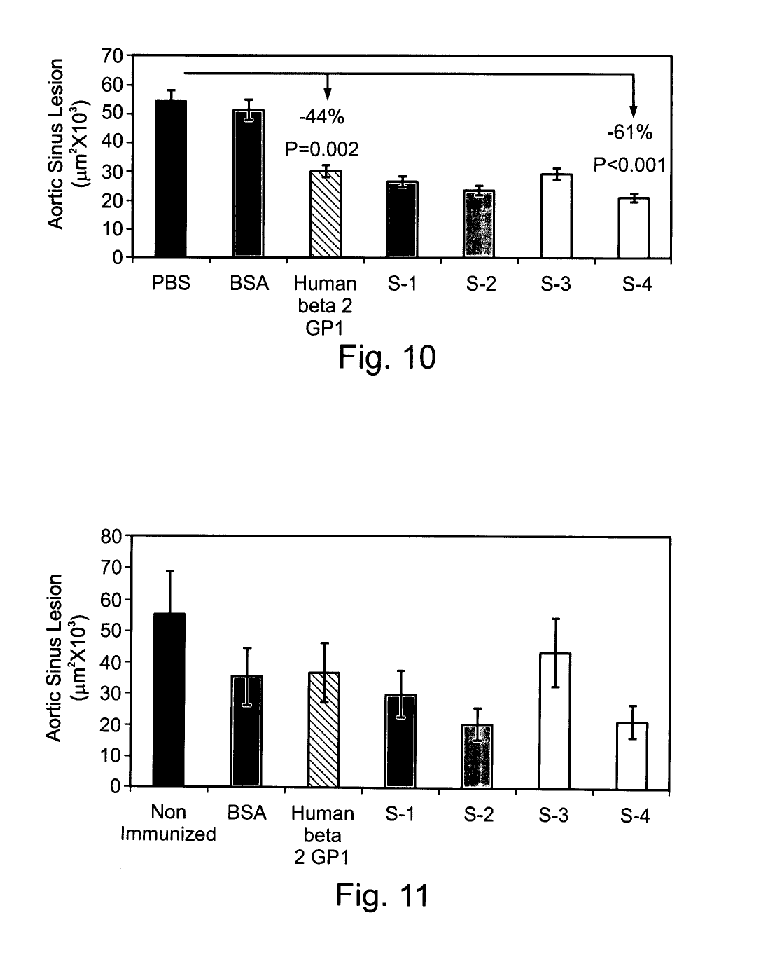

Oral administration of Human β₂GPI-derived peptides is specifically antiatherogenic in LDL-RD mice: Oral administration (via gavage, as described hereinabove) of human β₂GPI-derived peptides S-1 (SEQ ID NO: 11), S-2 (SEQ ID NO: 12), S-3 (SEQ ID NO: 13) and S-4 (SEQ ID NO: 14) at 100 μg/dose were approximately similarly effective in suppressing atherosclerosis (according to aortic sinus lesion size) in the LDL-RD mice (44% to 61% reduction, as compared with BSA- and PBS-fed controls), and all peptides are at least as effective as full length human β₂GPI (44% reduction, as compared with BSA- and PBS-fed controls) (see FIG. 10). Most effective was peptide S-4, which achieved a 61% reduction in aortic lesion size (p=0.001). Oral administration of BSA did not alter lesion progression in comparison with PBS (FIG. 10).

In order to rule out non-specific, systemic effects of oral administration of β₂GPI-derived peptides, the lipid profiles of the treated and control mice were determined.

Oral administration of Human β₂GPI-derived peptides does not significantly influence total cholesterol or serum triglyceride levels: Table 6 shows the results of oral administration of 100 μg human β₂GPI-derived peptides S-1, S-2, S-3, and S-4, or BSA, to LDL-RD mice, as described above. No significant influence of any of the human β₂GPI peptides administration, on total cholesterol or triglycerides levels was observed, indicating that the antiatherogenic effects of oral human β₂GPI-derived peptides administration do not result from alteration of the availability of plaque components. Thus, mucosal administration of β₂GPI-derived peptides results in specific inhibition of atherogenic processes.

TABLE 6
Lipid profile of mice tolerized orally with human β2GPI and human β2GPI-derived peptides

| | | | | Human | | | | | |
|---|---|---|---|---|---|---|---|---|---|
| | | | | β2 GPI | S-1 | S-2 | S-3 | S-4 | |
| | | PBS | BSA | 100 μg | 100 μg | 100 μg | 100 μg | 100 μg | |
| | | (n = 12) | (n = 12) | (n = 11) | (n = 12) | (n = 11) | (n = 11) | (n = 12) | Statistics* |
| Time | Weight (g) | 20.0 ± 0.7 | 20.0 ± 0.7 | 20.0 ± 0.7 | 20.6 ± 0.7 | 20.0 ± 0.7 | 19.6 ± 0.7 | 20.0 ± 0.7 | P = 0.976 |
| 0 | Cholesterol | 243 ± 7 | 247 ± 14 | 247 ± 9 | 244 ± 12 | 244 ± 14 | 241 ± 9 | 243 ± 11 | P = 1.000 |
| | (mg/dL) | | | | | | | | |
| | Triglyceride | 264 ± 23 | 248 ± 32 | 254 ± 27 | 238 ± 36 | 260 ± 28 | 283 ± 31 | 243 ± 31 | P = 0.955 |
| | (mg/dL) | | | | | | | | |
| End | Weight (g) | 29.2 ± 1.1 | 27.2 ± 1.4 | 26.5 ± 0.7 | 28.0 ± 0.7 | 26.8 ± 1.2 | 27.76 ± 0.7 | 26.6 ± 0.7 | P = 0.399 |
| | Cholesterol | 1286 ± 85 | 1164 ± 73 | 1130 ± 84 | 1104 ± 55 | 1148 ± 71 | 1172 ± 67 | 1181 ± 63 | P = 0.651 |
| | (mg/dL) | | | | | | | | |
| | Triglyceride | 262 ± 34 | 251 ± 24 | 295 ± 37 | 274 ± 33 | 382 ± 54 | 282 ± 29 | 345 ± 51 | P = 0.191 |
| | (mg/dL) | | | | | | | | |
| | Aortic sinus | 54275 ± 3649 | 54288 ± 6359 | 30250 ± 4464 | 26750 ± 4698 | 23636 ± 3857 | 29269 ± 3307 | 21125 ± 2482 | P < 0.001 |
| | lesion (μm²) | | | | | | | | |

*One way ANOVA (Mean ± S.E)

Immunization with Human β₂GPI-derived peptides mildly attenuates the early atherogenesis in LDL RD mice: In order to compare the effects of different modes of administration of human β₂GPI-derived peptides on atherosclerosis, human β₂GPI-derived peptides and human β₂GPI were administered intraperitoneally (IP) with incomplete Freund's adjuvant, in LDL RD mice, effectively immunizing the mice, as opposed to the tolerance effected by mucosal administration.

FIG. 11 shows the effect of repeated IP administration of 20 μg/dose of human β₂GPI-derived peptides or human β₂GPI, as compared with PBS in adjuvant and no immunization, on the extent of atherogenesis in LDL RD mice. Data presented in FIG. 11, and Table 7, clearly indicates a non-specific inhibitory effect of immunization (see PBS vs Non-immunized) on the size of the aortic sinus lesions in the susceptible LDL RD mice. On an average, immunization with the human β₂GPI-derived peptides was equally, if not more, effective in inhibiting atherogenesis as immunization with human β₂GPI, or with PBS prepared in incomplete Freund's adjuvant. In contrast to the protection achieved with mucosal administration, the different peptides S-1, S-2, S-3 and S-4 provided widely divergent, and diminished (34% or less) degrees of protection, as compared with controls. For example, intraperitoneal immunization with the peptide S-3 was without any significant anti-atherogenic effect as compared to PBS+IFA, whereas mucosal administration of the same S-3 peptide resulted in clearly effective inhibition of atherogenesis (FIG. 10 and Table 6, compared to Table 7).

TABLE 7
Lipid profile of LDL-RD mice immunized intraperitoneally with human β₂GPI peptides and incomplete Freund's adjuvant.

| | | | | Human | | | | | |
|---|---|---|---|---|---|---|---|---|---|
| | | | | β2 GPI | S-1 | S-2 | S-3 | S-4 | |
| | | Non | PBS | 100 μg | 100 μg | 100 μg | 100 μg | 100 μg | |
| | | (n = 8) | (n = 9) | (n = 11) | (n = 8) | (n = 9) | (n = 8) | (n = 9) | Statistics |
| Time | Weight | 19.9 ± 1.2 | 20.0 ± 1.1 | 19.9 ± 1.0 | 20.7 ± 1.2 | 20.2 ± 1.2 | 19.9 ± 0.9 | 20.0 ± 1.0 | P = 0.976 * |
| 0 | (g) | | | | | | | | |
| | Choles- | 189 ± 17 | 184 ± 9 | 202 ± 11 | 192 ± 11 | 196 ± 9 | 201 ± 12 | 194 ± 10 | P = 1.000 * |
| | terol | | | | | | | | |
| | (mg/dL) | | | | | | | | |
| | Triglyc- | 77 ± 3 | 68 ± 9 | 77 ± 11 | 67 ± 8 | 80 ± 8 | 78 ± 18 | 68 ± 6 | P = 0.955 * |
| | eride | | | | | | | | |
| | (mg/dL) | | | | | | | | |
| | Weight | 30.6 ± 1.3 | 31.5 ± 1.4 | 30.6 ± 1.1 | 32.5 ± 0.8 | 30.5 ± 0.7 | 29.2 ± 0.9 | 28.8 ± 1.3 | P = 0.399 * |
| | (g) | | | | | | | | |
| End | Choles- | 1364 ± 145 | 1135 ± 86 | 1319 ± 136 | 1380 ± 74 | 1289 ± 89 | 1385 ± 123 | 1267 ± 59 | P = 0.651 * |
| | terol | | | | | | | | |
| | (mg/dL) | | | | | | | | |
| | Triglyc- | 381 | 537 | 315 | 417 | 576 | 459 | 321 | P = 0.227 ** |
| | eride | | | | | | | | |
| | (mg/dL) | | | | | | | | |
| | Aortic | 55239 ± 9571 | 35613 ± 6865 | 36782 ± 5962 | 29968 ± 6032 | 24050 ± 7755 | 45329 ± 14667 | 23758 ± 5767 | P < 0.001 * |
| | sinus | | | | | | | | |
| | lesion | | | | | | | | |
| | (μm²) | | | | | | | | |

* One way ANOVA (Mean ± S.E)
** Kruskal-Wallis One Way Analysis of Variance on Ranks (Median)

Without wishing to be limited to a single hypothesis, it will be appreciated that the inconsistent effects of IP immunization with human β₂GPI-derived peptides, and human β₂GPI observed in FIG. 11 are most likely the result of non-specific reaction to adjuvant administration.

Thus, the results presented hereinabove clearly show that mucosal administration of human β₂GPI-derived peptides is effective in specifically inhibiting atherogenesis, and provides superior anti-atherogenic protection, compared to other modes of administration, such as intraperitoneal immunization.

### Example 8

### Superior Inhibition of Atherogenesis in Genetically Predisposed (apoE-Deficient) Mice by Induction of Mucosal Tolerance with Mucosal Administration of β₂Gpi-Derived Peptides

Oral administration of Human β₂GPI derived peptides specifically inhibits progression of advanced atherogenic processes in ApoE-KO mice: Adult ApoE-KO mice develop advanced atherosclerotic lesions when fed an atherogenic “Western diet”. In order to determine the protective effect of mucosal administration of β₂GPI-derived peptides on the later stages of atherosclerotic development, adult ApoE-KO mice were treated with oral administration of human β₂GPI-derived peptide S-4 (SEQ ID NO: 14), which provided the most effective inhibition of early atherogenesis in mucosal administration to LDL RD mice (see Example 7, and FIG. 10, hereinabove). 10-11 week-old male ApoE-KO mice, having early atherosclerotic lesions, were treated with 5 oral administrations (by gavage, as described hereinabove) given every other day of human β₂GPI peptide S-4 (SEQ ID NO: 14) or human β₂GPI (50 μg/dose) or PBS (0.2 ml). Lesion areas calculated from cryosections of the aortic sinus were compared between control untreated, human β₂GPI treated and S-4 human β₂GPI-derived peptide treated mice at 8 weeks following the last oral administration of the antigens, or of PBS.

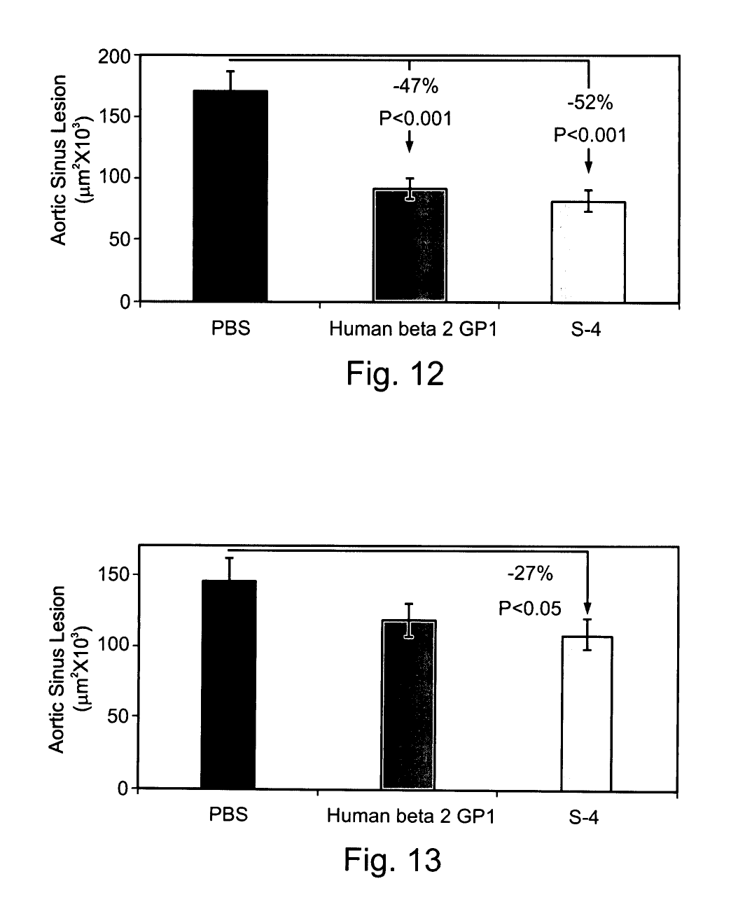

It will be appreciated, that the aortic lesions in the ApoE-KO mice are significantly more extensive than those of the LDL RD mice (see Example 6, FIG. 9 for comparison), representing severe atherogenic involvement of the aortic sinuses. As shown in FIG. 12, oral administration of 50 μg human β₂GPI-derived peptides followed by 8 weeks of chow diet, inhibited the progression of atherosclerotic lesions (52% reduction compared to PBS controls), with greater effectivity than oral administration of human β₂GPI (47% reduction compared to PBS controls).

In order to rule out non-specific, systemic effects of oral administration of β2GPI-derived peptides, the lipid profiles of the treated and control mice were determined.

Oral administration of Human β₂GPI-derived peptides does not significantly influence metabolic profile of mice having advanced atherosclerosis: Table 8 shows the results of oral administration of 50 μg of human β₂GPI, human β2GP-derived peptide S-4, or PBS, to adult male Apo-E KO mice, as described above. Table 8 clearly shows that oral administration of human β₂GPI peptide S-4 is effective in inhibiting advanced atherosclerosis in the ApoE-KO mice, despite a mild elevation of triglyceride levels (107±4 mg/Dl vs 87±4 mg/Dl for PBS treated controls) in the human β2GPI-derived peptide-treated group. No significant influence of oral β₂GPI-peptide administration, on body weight or total cholesterol was observed, indicating that the inhibition of atherosclerotic progression in adult male Apo-E KO by oral β₂GPI-derived peptide administration does not result from alteration of the availability of plaque components or lipid metabolism.

TABLE 8
Lipid profile of ApoE-KO mice orally tolerized
with human β2GPI-derived peptides.

| | | | Human | Peptide | |
|---|---|---|---|---|---|
| | | | β2 GPI | S-4 | |
| | | PBS | 50 μg | 50 μg | |
| | | (n = 15) | (n = 13) | (n = 13) | Statistics* |
| Time 0 | Weight (g) | 24.7 ± 0.7 | 24.5 ± 0.7 | 24.8 ± 0.9 | P = 0.948 |
| | Cholesterol | 383 ± 15 | 377 ± 24 | 384 ± 18 | P = 0.958 |
| | (mg/dL) | | | | |
| | Triglyceride | 84 ± 4 | 86 ± 5 | 85 ± 5 | P = 0.952 |
| | (mg/dL) | | | | |
| End | Weight (g) | 26.2 ± 0.6 | 25.6 ± 0.5 | 25.4 ± 0.9 | P = 0.587 |
| | Cholesterol | 382 ± 18 | 339 ± 11 | 414 ± 20 | P = 0.375 |
| | (mg/dL) | | | | |
| | Triglyceride | 87 ± 5 | 78 ± 3 | 107 ± 4 | Pep-14/pbs |
| | (mg/dL) | | | | P = 0.01** |
| | Aortic sinus | 170962 ± 12598 | 90833 ± 9502 | 81923 ± 9191 | P < 0.001 |
| | lesion (μm²) | | | | |

Nasal administration of Human β₂GPI derived peptides specifically inhibits progression of advanced atherogenic processes in ApoE-KO mice: The membranous tissue around the eyes, the middle ear, the respiratory and other mucosa, and especially the mucosa of the nasal cavity, like the gut, possess mechanisms for immune reactivity. Thus, Rossi, et al (Scand J Immunol 1999 August; 50(2):177-82) found that nasal administration of gliadin was as effective as intravenous administration in downregulating the immune response to the antigen in a mouse model of celiac disease. Similarly, nasal exposure to acetylcholine receptor antigen was more effective than oral exposure in delaying and reducing muscle weakness and is specific lymphocyte proliferation in a mouse model of myasthenia gravis (Shi, F D. et al, J Immunol 1999 May 15; 162 (10): 5757-63). Therefore, in addition to oral administration, immunogenic compounds intended for mucosal administration should be adaptable to nasal and other membranous routes of administration.

Indeed, as shown in Example I hereinabove (see FIG. 1), nasal administration of plaque antigens HSP-65, OxLDL and β₂GPI resulted in significant inhibition of early atherogenesis in LDL RD mice. In order to determine the protective effect of nasal administration of β2GPI-derived peptides on the later stages of atherosclerotic development, adult ApoE-KO mice were treated with nasal administration of human β₂GPI-derived peptide S-4 (SEQ ID NO: 14) as described in the Methods section above. Briefly, three doses of human β2GPI, human β2GPI peptide S-4 (SEQ ID NO: 14) or human β2GPI (10 μg/dose) or PBS (0.2 ml), suspended in PBS, were administered intranasally every other day to 11-13 week-old male ApoE-KO mice, having early atherosclerotic lesions. Lesion area was calculated from cryosections of the aortic sinus were compared between control untreated, human β2GPI treated and S-4 human β2GPI-derived peptide treated mice at 8 weeks following the last oral administration of the antigens, or of PBS.

As shown in FIG. 13, nasal administration of 10 μg human β2GPI-derived peptides followed by 8 weeks of chow diet, inhibited the progression of atherosclerotic lesions (27% reduction compared to PBS controls), with greater effectivity than nasal administration of human β2GPI (19% reduction compared to PBS controls).

In order to rule out non-specific, systemic effects of nasal administration of β2GPI-derived peptides, the lipid profiles of the treated and control mice were determined.

Nasal administration of Human β₂GPI-derived peptides does not significantly influence metabolic profile of mice having advanced atherosclerosis: Table 9 shows the results of nasal administration of 10 μg of human β₂GPI, human β₂GPI-derived peptide S-4, or PBS, to adult male Apo-E KO mice, as described above. Table 9 clearly shows that oral administration of human β2GPI peptide S-4 is effective in inhibiting advanced atherosclerosis in the ApoE-KO mice. No significant influence of nasal β₂GPI-peptide administration, on body weight, triglycerides or total cholesterol was observed, indicating that the inhibition of atherosclerotic progression in adult male Apo-E KO by nasal β₂GPI-derived peptide administration does not result from alteration of the availability of plaque components or lipid metabolism.

TABLE 9
Lipid profile of ApoE-KO mice nasally tolerized with human β₂GPI- derived peptides.

| | | Groups | | | |
|---|---|---|---|---|---|
| | | | Human | Peptide | |
| | | | β2 GPI | S-4 | |
| | | PBS | 10 μg/mouse | 10 μg/mouse | |
| Schedule | Parameter | (n = 14) | (n = 15) | (n = 15) | Statistics* |
| Time 0 | Weight (g) | 20.6 ± 0.5 | 20.0 ± 0.5 | 20.5 ± 0.4 | P = 0.993 |
| | Cholesterol | 353 ± 15 | 357 ± 13 | 356 ± 12 | P = 0.978 |
| | (mg/dl) | | | | |
| | Triglyceride | 69 ± 5 | 67 ± 5 | 66 ± 5 | P = 0.947 |
| | (mg/dl) | | | | |
| End | Weight (g) | 25.7 ± 0.6 | 25.5 ± 0.4 | 25.7 ± 0.5 | P = 0.959 |
| | Cholesterol | 323 ± 16 | 308 ± 9 | 323 ± 25 | P = 0.782 |
| | (mg/dl) | | | | |
| | Triglyceride | 120 ± 7 | 108 ± 4 | 117 ± 7 | P = 0.348 |
| | (mg/dl) | | | | |
| | Aortic sinus | 1474242 ± 15476 | 117449 ± 7524 | 107953 ± 10158 | P < O.05 |
| | lesion (μm²) | | | | |

*One way ANOVA (Mean ± S.E)

Thus, the results presented hereinabove show that nasal administration of human β₂GPI-derived peptides effectively and specifically protects atherogenically prone ApoE-KO mice from advanced atherosclerotic plaquing, to an extent consistently superior to the protection afforded by whole β₂GPI.

### Example 9

### Mucosal Administration of β₂GPI-Derived Peptides Specifically Suppresses the Immune Response to β₂GPI and Other Plaque-Related Autoantigens in LDL-Receptor Deficient Mice

Nicoletti et al (Mol. Med. 2000; 6, 283-90) have shown that tolerance to the antigens in oxidized LDL, brought about by neonatal administration of β₂GPI, led to clonal anergy/deletion of the oxLDL reactive cells and to consequent suppression of atherosclerosis. While reducing the present invention to practice, it was uncovered that oral and nasal administration of β₂GPI to LDL-RD mice inhibited the proliferation response to β₂GPI in antigen-sensitized immune cells (lymph nodes) (see, for example, Example 3, Table 3 hereinabove), and also inhibited proliferation of OxLDL-sensitized immune (lymph nodes) (see Example 6, FIG. 7B). Thus, the effect of mucosal administration of β₂GPI-derived peptides on the character of the immune response to plaque related autoantigens was assessed in LDL-RD mice.

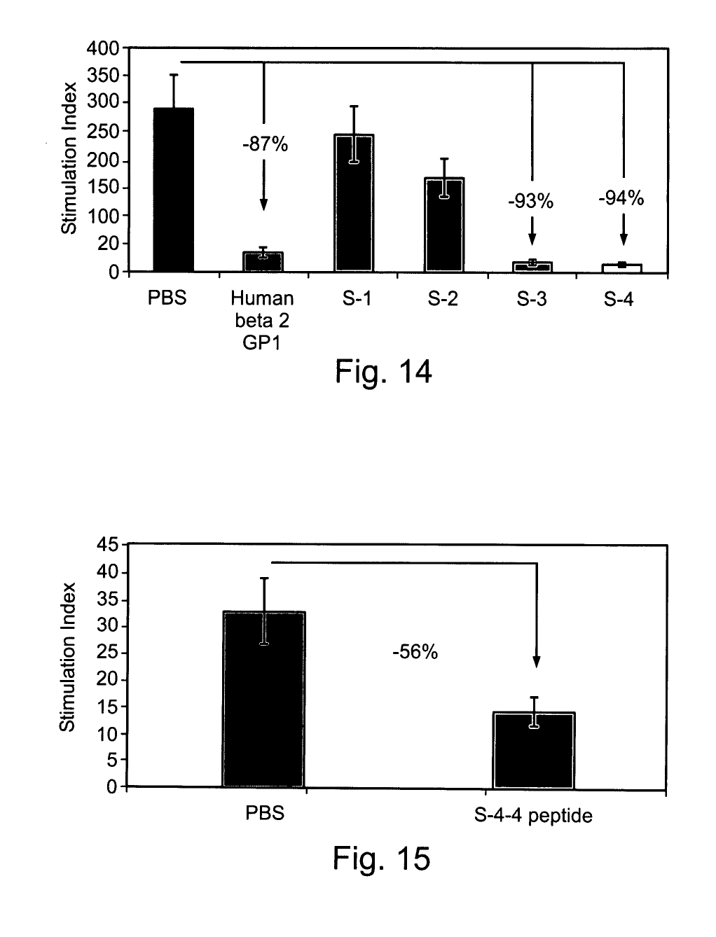

Oral administration of β₂GPI-derivedpeptides inhibits the cellular immune response to plaque related antigens: In order to assess the role of specific induction of immune tolerance in the antiatherogenic effects of oral administration of β₂GPI-derived peptides to LDL-RD mice, the extent of lymph node proliferation in response to challenge with OxLDL was compared in LDL-RD mice receiving oral β₂GPI-derived peptides S-1, S-2, S-3, or S-4, oral β₂GPI or BSA administration. 8 week old female LDL-RD mice received three doses of human β₂GPI-derived peptides S-1, S-2, S-3, or S-4, oral β₂GPI or BSA, as indicated, were immunized with 0xLDL in incomplete Freund's adjuvant, and inguinal lymph node cells proliferation in response to 0xLDL challenge was assessed by thymidine uptake. FIG. 14 shows the differences in thymidine uptake, expressed as Stimulation Index, between lymph node cells from LDL-RD mice immunized with OxLDL, following oral administration of β₂GPI-derived peptides, or BSA, and exposure of the cells to the sensitizing antigen.

Oral administration of β₂GPI-derived peptides effectively inhibits the cellular immune response to the plaque related antigens in sensitized mice. FIG. 14 shows the significant inhibition of lymph node cell proliferation stimulated by β₂GPI-derived peptides in the OxLDL tolerized mice. While oral administration of peptides S-1 and S-2 mildly reduced the Stimulation Index of sensitized lymph node cells in response to OxLDL, oral administration of β₂GPI-derived peptides S-3 and S-4 dramatically inhibited the response of OxLDL-sensitized immune (lymph node) cells (93 and 94%, respectively), both greater than the reduction in Stimulation Index observed with oral administration of β₂GPI (87%) (Table 10). Thus, oral administration of β₂GPI-derived peptides S-1, S-2, S-3 and S-4 results in specific suppression of the primary cellular immune response to plaque antigens other than β₂GPI (OxLDL), with the strongest suppression induced by β₂GPI-derived peptides S-3 and S-4 (Table 10).

TABLE 10
Effect of mucosal administration of β₂GPI-derived
peptides on cellular immune response to OxLDL.

| | Group | | | | | | |
|---|---|---|---|---|---|---|---|
| | PBS | H-β2 GPI | S-1 | S-2 | S-3 | S-4 | Statistics |
| Stimulation | 290 ± 6 | 38 ± 12 | 246 ± 79 | 168 ± 72 | 20 ± 6 | 17 ± 10 | P < 0.005 |
| index | | | | | | | |

Data presented as Mean ± S.E. and statistical analysis performed using t-test.

S-4-derived peptides inhibit the cellular immune response to plaque related antigens: In order to identify specific sequences of the β₂GPI-derived peptides conferring mucosal tolerance-inducing activity, sequential synthetic overlapping 12-mer peptides representing the entire sequence of β2GPI-derived peptide S-4 (designated peptides S-4-1, S-4-2, S-4-3 . . . S-4-10, SEQ ID NOs: 15 and 24, respectively) were assessed for inhibition of cellular immune response to Ox LDL, as described hereinabove.

6.5-week old female LDL-RD mice received oral administration of 5 doses (100 μg/dose) of β2GPI-derived peptides S-4-1, S-4-3, S-4-5, S-4-6, S-4-7, S-4-8, S-4-9 or S-4-10 (SEQ ID NOs: 15-24, respectively), or PBS control. Three days after the final administration of peptides, the mice were immunized with OxLDL in incomplete Freund's adjuvant, as described in the Examples above, and lymph node cells assessed for antigen (OxLDL) stimulation of proliferation 10 days later. Proliferation was assessed by incorporation of thymidine, expressed as the Stimulation Index, as described.

FIG. 15 shows that oral administration of the β₂GPI-derived peptide S-4-4 (SEQ ID NO: 18) was effective in suppressing the sensitized T-cell response to stimulation by another plaque-related antigen, OxLDL, decreasing the Stimulation Index by nearly 60%, as compared with PBS-treated controls. Thus, the results presented herein indicate that mucosal administration of β₂GPI-derived peptides, or portions thereof, effectively suppresses primary T-cell responses towards plaque antigens other than β₂GPI. Without wishing to be limited by a single hypothesis, such heterologous suppression of T-cell response to plaque antigens can be the result of “bystander” effects of mucosal administration of β₂GPI-derived peptides, as discussed hereinabove.

Thus, the results brought herein show, for the first time, that mucosal administration (oral, nasal, etc) of human β₂GPI-derived peptides is capable of suppressing both early and late atherogenic processes in genetically susceptible mice, more effectively than full-length β₂GPI or immunization with β₂GPI or β₂GPI-derived peptides. Further, the results show, for the first time, that prior mucosal administration with β₂GPI-derived peptides (especially S-4 and S-4-4) significantly inhibits reactive T-cell proliferation in lymph node cells from mice immunized with plaque antigens such as OxLDL.

Thus, the results presented hereinabove demonstrate, for the first time, that mucosal administration of β₂GPI-derived peptides can be effective in attenuating fatty streak formation, modulating plaque-related immune response, and inhibiting the progression of early and advanced atherosclerotic lesions.

### Example 10

### Mucosal Administration of Combined 8₂GPI-Derived Peptides

Examples 7-9 show that mucosal administration of β₂GPI-derived peptides representing different portions of the amino acid sequence of the human β₂GPI polypeptide results in a range of effective antiatherogenic activity, indicating that the component peptide sequences of β₂GPI can comprise individually effective β₂GPI-derived peptides having unique antiatherogenic activity. Such component antiatherogenic peptides, when administered in combination, can produce a synergic therapeutic effect, greater than the sum of the effects of each individual β₂GPI-derived peptide.

Examples of such synergy of combination of peptides are well known in the art. Multiple antigenic peptides have been identified in the pathogenesis and prevention of autoimmune NOD-diabetes (Judkowski et al Clin Immunol 2004, 113:29-37; and Casares et al., Curr Mol Methods 2001; 1:357-378); grass-pollen allergy (Zhang et al Immunology 1996; 87:283); coeliac disease (Seisser et al Clin Exper. Immunol 2001:125:216); pemphigous (Harman et al J Dermatol. 2000; 142:1135-39; and Lafitte et al Brit Jour. Dermatol 2001; 144: 760); and vitiligo (Kemp et al. J Invest. Dermatol. 1999; 113:267).

Multivalent antigen peptides corresponding to divalent, trivalent and tetravalent combinations of synthetic peptide epitopes selected from a hexapeptide library screened with anti-β₂GPI antibodies have been disclosed by Blank et al (U.S. Pat. No. 6,825,319, PCT filed Jul. 6, 1999). Blank et al. have demonstrated that administration of the divalent and tetravalent peptide conjugates of the synthetic epitopes recognized by anti-PL serum were capable of inhibiting antibody secretion by peripheral immune cells of aPL patients. In vitro exposure of B-cell populations enriched for anti-β2GPI forming cells to the 12-mer synthetic peptide A, and the 14-mer synthetic peptide B together (see Example 10 of Blank et al) resulted in a significant synergic inhibition of IgG and IgM antibody forming cell (AFC) activity in these cells. However, Blank et al. did not demonstrate any similar activity of human β₂GPI-derived peptides, since BLAST analysis of the amino acid sequence of peptide A indicated that there was no similarity between human β₂GPI and peptide A. BLAST analysis of the amino acid sequence of peptide B using the same parameters showed a limited similarity (81%) (between domain 5 of the human β₂GPI and peptide B), between a portion of peptide B and amino acids 227-237 of the mature β₂GPI.

Victoria et al. (U.S. patent application Ser. No. 10/044,844, and Jones et al Biocong. Chem. 1999; 10:480-88; and Jones et al. Biocong Chem 2001; 12:1012-20) have disclosed synthetic peptides recognized by sera from aPL patients, identified by phage display library screening, for use as B-cell tolerizing agents. The synthetic peptides, some of which were recognized by anti-human β₂GPI antibodies, showed no sequence similarity with human β₂GPI polypeptide when analyzed by BLAST analysis.

Krause et al (Cutting Edge Peptides, www.rheuma21st.com) have also disclosed immune active synthetic peptides comprising epitopes recognized by anti-human β₂GPI antibodies, including the peptides taught by Blank et al (see above), with the addition of a third sequence, also having no homology to human β₂GPI sequences, according to BLAST analysis.

Thus, although peptides and chimeric peptides comprising synthetic anti-human β₂GPI antibody epitopes have been tested alone and in combination, none of the cited prior art teaches or motivates to mucosal administration of human β₂GPI peptides, and compositions comprising same, individually or in combination, for inducing mucosal tolerance to human β₂GPI, and the treatment and/or prevention of atherosclerosis.

In order to assess the anti-atherogenic activity of mucosal administration of human β2GPI peptides in combination, activity of the human β2GPI peptide combinations is first assayed for induction of anti-inflammatory cytokines, and suppression of pro-inflammatory cytokines.

Combined β2GPI-derived Peptides modulate the immune response to β2GPI in aortic sinus tissue: Combined β₂GPI-derived peptides, including admixtures of at least two human β₂GPI-derived peptides, and chimeric peptides of at least two β₂GPI-derived peptides covalently linked are prepared as described in detail in the General Materials and Methods section hereinabove. Briefly, antigenic peptides derived from human β₂GPI (SEQ ID NO: 10) are prepared from native, purified human, recombinant and/or synthetic β₂GPI by, for example, proteolytic digestion, chemical fragmentation, mechanical fragmentation, etc; or antigenic peptides derived from human β₂GPI are synthesized according to standard peptide synthesis protocol, essentially as described by Ito et al (Hum Immunol 2000; 61:366-377), or by Blank et al (PNAS USA 1999; 96:5164-5168); or cloned and expressed in transformed cells or organisms as recombinant peptides, according to published protocols, as described hereinabove, and by Iverson et al. (PNAS 1998; 95:15542-46) in detail. Examples of suitable β2GPI peptides are as set forth in SEQ ID NOs. 25-57315.

Examples 7-9 hereinabove revealed the anti-atherogenic activity of human β₂GPI-derived peptides S-1, S-2, S-3 and S-4, and of S-4-4, in individual mucosal administration. In order to assess the synergic effects of administration of the peptides in combination, oral and nasal administration of admixtures representing all possible combinations of at least two of the peptides (for example, S-1+S-2; S-1+S-3; S-1+S-4; S-1+S-4-4; S-2+S-3; S-2+S-4; S-2+S-4-4; S-3+S-4; S-3+S-4-4; S-4+S-4-4; and similar permutations of 3 and 4 and 5 peptides), 10-1000 μg/mouse as detailed hereinabove, is effected in male ApoE-KO mice 7-9 weeks of age, in 2-10 administrations, on every other day. Three days following the oral administration of β2GPI-derived peptides, the mice are sacrificed, and aortas collected and processed for RT-PCR analysis of the expression of anti-inflammatory Th2 type cytokines IL-10 and IL-4, and the proinflammatory Th1-type cytokines IFN-γ and TGF-β, as described hereinabove. Following the RT-PCR reactions the cytokine transcripts are separated for visualization and quantification by SDS-PAGE. Specific induction of the anti-inflammatory IL-4 and/or IL-10 cytokines, and suppression of IFN-γ and/or TGF-β expression in the aortic tissue of the treated mice, without overall induction of housekeeping gene (such as β-actin) expression indicates the modulation of immune reactivity in the atheroma tissue. Comparison of the extent and pattern of modulation between the various combinations of the β₂GPI-derived peptides indicates the β₂GPI-derived peptides having synergic effects greater or different than the effects observed for individual peptides.

Having identified the combined β₂GPI-derived peptides demonstrating synergic anti-inflammatory activity in aortic sinus of atherosclerosis-prone Apo E KO mice, further assessment of the anti-atherogenic effect of combined β₂GPI-derived peptides can be performed.

Oral administration of combined β₂GPI-derived peptides in LDL-RD and Apo E KO mice: Oral administration (via gavage, as described hereinabove) of human β₂GPI-derived peptides representing combinations of at least two β₂GPI-derived peptides, as described hereinabove at 10-1000 μg/dose is effected in LDL-RD mice, using BSA and PBS as controls, in 2-10 doses over a period of 2-4 weeks, followed by an atherogenic, “Western” diet.

Similarly, the effects of combined β2GPI-derived peptides on advanced atherosclerotic lesions are assessed in the Apo E KO mouse model. In this series of experiments, oral administration of combined β2GPI-derived peptides (via gavage, as dscribed hereinabove) is effected in 8-14 week old Apo E KO mice, using BSA and PBS controls, in 2-10 doses over a period of 2-4 weeks. 6-10 weeks following the last administration, the mice are sacrificed, and the aortic sinus lesions evaluated.

The extent of aortic sinus lesion is assessed from Oil-red O stained cryosections of the aortic sinus, as described hereinabove. Reduction of the severity and extent of aortic sinus lesions in the early-atherogenic model LDL-RD, and/or in the late stage atherogenic model Apo E KO, as compared to equal quantities of the individual β₂GPI-derived peptides indicates which combinations of β₂GPI derived peptides are synergic in their atherogenic activity in mucosal administration.

Nasal Administration of Combined β₂GPI Derived Peptides on Atherogenic Processes in LDL-RD and Apo E KO Mice:

As shown in Examples 1 and 8 hereinabove (see FIG. 1), nasal administration of plaque antigens HSP-65, OxLDL and β₂GPI, and β₂GPI-derived peptides results in significant inhibition of early atherogenesis in LDL RD and Apo E KO mice. In order to determine the protective effect of nasal administration of combined β₂GPI-derived peptides on the earlier and later stages of atherosclerotic development, adult LDL-RD and ApoE-KO mice are treated with nasal administration of combined β₂GPI-derived peptides as described in the Methods section above. Briefly, three doses of combined human β₂GPI (10 μg/dose), human β₂GPI (10 μg/dose) or PBS (0.2 ml), suspended in PBS, are administered intranasally every other day to 8-12 week old LDL-RD mice or 11-13 week-old male ApoE-KO mice. The mice are then fed either a chow diet (Apo E KO) or the atherogenic “Western” diet (LDL RD), and sacrificed 5-10 weeks later. Lesion area calculated from cryosections of the aortic sinus is then compared between control untreated, human β₂GPI treated and combined β2GPI-derived peptide treated mice following the oral administration of the antigens, or of PBS. Reduction of the severity and extent of aortic sinus lesions in the early-atherogenic model LDL-RD, and/or in the late stage atherogenic model Apo E KO, as compared to equal quantities of the individual β₂GPI-derived peptides indicates which combinations of β₂GPI derived peptides are synergic in their atherogenic activity in mucosal administration.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by their accession number was specifically and individually indicated to be incorporated herein by reference. In addition, citation or identification of any reference in this application shall not be construed as an admission that such reference is available as prior art to the present invention.

CD-ROM Content

Enclosed herewith and filed with the application is a CD-ROM, containing file(s) as listed herein. File information is provided as: File name/byte size/date of creation/operating systems/machine format.

CD-ROM:

28360.5T25/65,594,037/Mar. 15, 2005/PC/Notepad

### REFERENCES

### Additional References are Cited in the Text

Afek A et al. Pathobiology 1999; 67:19-25.
Altman, Thrombosis J. 2003; 1:4.
Ameli S, et al. Arterioscler Thromb Vasc Biol 1996; 16: 1074-1079.
Becker et al. PNAS USA 1997; 94:10873-78.
Bili, et al. Circulation, 2000; 102:1258.
Bimie DH Eur Heart J 1998; 19:366-67.
Brey, et al Stroke 2001; 32:1701-06.
Cabral A R et al Am J Med 1996; 101:472-81.
Caligiuri et al, J Clin Invest, 2002, 109:745-53.
Chonn A, et al J Biol Chem 1995; 270: 25845-49.
Collins, et al., Infect Immun 2002; 70:2282-87.
Erkkila et al Atherosclerosis 2000; 20:204-9.
Faxon et al, Circulation 2004; 109:2617-25.
Freigang, et al. 1998; 1972-82.
George J, et al Rheum Dis Clin North Am 2001; 27:603-10.
George et al, Atherosclerosis, 1998, 138; 147-152
George J, et al Circulation 1998; 15:1108-15
George J, et al. Immunol Res 1996; 15:315-322, 1996.
George et al., Cardiovascular Research 2004, 62:603-609
Gotesman et al, J Pharma and Expanding Ther. 2001; 297-32.
Greaves at el, Trends in Immunol. 2002; 23:535-41.
Gromadzka G, et al Cerebrovasc Dis 2001; 12:235-39.
Hajjar, D P and Haberland, M E, J. Biol Chem 1997 Sep. 12; 272(37):22975-78.
Halperin G et al Methods in Enzymology 129, 838-846, 1986.
Hanninen et al., Immunol Rev 2000; 173:109-19.
Koike, et al (Ann Med 2000; 32:Suppl I 27-31.
Kyobashi, et al (J Lipid Res 2001; 42:697-709).
Libby P, Hansson GK. Lab Invest 1991; 64: 5-15.
Libby, Nature 2002; 420:868-74.
Limaye et al Aust and New Zeal J of Med, 1999; 29.
Levine et al JAMA, 2004; 291:576-84.
Manzi, Rheumatology 2000; 39:353-359.
Martinez-Gonzales et al, Rev Esp Cardiol, 2001; 54:218-31.
Paigen B, et al. Atherosclerosis 1987; 68: 231-140.
Palinski W, et al. Arteriosclerosis 1990; 10: 325-335.
Palinski W, et al. Arterioscler Thromb 1994; 14: 605-616
Palinski W, et al. Proc Natl Acad Sci USA. 1995; 92: 821-825.
Peirangeli et al J Autoimmunity 2004; 22:217-25.
Plump A S, et al. Cell 1992; 71: 343-353.
Roselaar S E et al. Arterioscler Thromb Vasc Biol 1996; 16: 1013-1018.
Ross R. Nature 1993; 362: 801-809.
Ross, Atherosclerosis 1997, 131 Suppl.:S3-7.
Rubin E M. Et al. Nature 1991; 353: 265-267.
Sadovsky, Amer Fam Phys December 1999.
Schachter, Int J Card 1997; 62, Suppl. 2:S3-7.
Segovia, J of Rheumatology; Hatori et al. Arthritis Rheum. 2000; 43:65-75.
Steinberg D, et al. N Engl J Med 1989; 320: 915-924.
Takeda, et al., Stroke 2002; 33:2156.
Thiagarajan P, et al Arterioscler Thromb Vasc Biol 1999; 19:2807-11
Uyemura K et al J Clin Invest 1996 97; 2130-2138.
Watson A. D., et al. J. A. J. Biol. Chem. 272:13597-13607, 1997.
Weiner H, et al. Annu Rev Immunol 1994; 12: 809-837.
Wick G, et al. Immunol Today 1995; 16: 27-33.
Witztum J and Steinberg, D, Trends Cardiovasc Med 2001 April-May; 11(3-4):93-102.
Witztum J. Lancet 1994; 344: 793-795.
Zhang et al. Science 1992; 258: 468-471.
Zhou et al Exp Opin Biol Ther 2004; 4:599-612.
Zhou et al, Arterioscler Thromb Vasc Biol 2001; 21:108-14.
Xu Q, et al Circulation 1999; 100:1169-74.

1.-65. (canceled)

66. A method for prevention and/or treatment of a vascular condition in a subject in need thereof comprising administering to a mucosal surface of the subject a mucosal tolerance-inducing amount of at least one beta₂-glycoprotein-1 (β₂GPI)-derived peptide, thereby inducing mucosal tolerance.

67. The method of claim 66, wherein said at least one beta₂-glycoprotein-1 (β₂GPI)-derived peptide comprises a combination of at least two beta₂-glycoprotein-1 (β₂GPI)-derived peptides.

68. The method of claim 66, wherein said at least one β₂GPI-derived peptide is a human β₂GPI-derived peptide.

69. The method of claim 66, wherein said at least one β₂GPI-derived peptide is a synthetic peptide.

70. The method of claim 66, wherein said at least one β₂GPI-derived peptide has a sequence as set forth in one of SEQ ID NOs: 25-57,315.

71. The method of claim 67, wherein said combination of at least two β₂GPI-derived peptide is a chimeric peptide comprising at least two β₂GPI-derived peptides in covalent linkage.

72. The method of claim 71, wherein said chimeric peptide comprises a first β₂GPI-derived peptide having a sequence as set forth in one of SEQ ID NOs: 25-57,315 covalently linked to a second β₂GPI-derived peptide having a sequence as set forth in any of SEQ ID NOs: 25-57,315.

73. The method of claim 66, further comprising administering a therapeutically effective amount of at least one additional compound selected from the group consisting of HMGCoA reductase inhibitors (statins), mucosal adjuvants, corticosteroids, anti-inflammatory compounds, analgesics, growth factors, toxins, and additional tolerizing antigens.

74. The method of claim 66, wherein said vascular condition is selected from the group consisting of atherosclerosis, cardiovascular disease, cerebrovascular disease, peripheral vascular disease, stenosis, restenosis and/or in-stent-stenosis.

75. The method of claim 66, wherein said mucosal tolerance results in modulation of an immune response to a beta₂-glycoprotein-1 (β₂GPI).

76. A method for modulating an immune response to an atheroma plaque-related antigen in a subject in need thereof comprising administering to a mucosal surface of the subject a mucosal tolerance-inducing amount of an active ingredient comprising at least one beta₂-glycoprotein-1 (β₂GPI)-derived peptide thereby inducing mucosal tolerance and modulating the immune response to the atheroma plaque-related antigen.

77. The method of claim 76, wherein said at least one beta₂-glycoprotein-1 (β₂GPI)-derived peptide is a combination of at least two beta₂-glycoprotein-1 (β₂GPI)-derived peptides.

78. A pharmaceutical composition for prevention and/or treatment of a vascular condition in a subject in need thereof comprising as an active ingredient a combination of at least two beta₂-glycoprotein-1 (β₂GPI)-derived peptides and a pharmaceutically acceptable carrier.

79. The pharmaceutical composition of claim 78, wherein said at least two β₂GPI-derived peptides are human β₂GPI-derived peptides.

80. The pharmaceutical composition of claim 78, wherein at least one of said at least two β₂GPI-derived peptides is a synthetic peptide.

81. The pharmaceutical composition of claim 78, wherein said at least two β₂GPI-derived peptides have a sequence as set forth in one of SEQ ID NOs: 25-57,315.

82. The pharmaceutical composition of claim 78, wherein said combination of at least two β₂GPI-derived peptides is a mixture of peptides.

83. The pharmaceutical composition of claim 78, wherein said combination of at least two β₂GPI-derived peptides is a chimeric peptide comprising at least two β₂GPI-derived peptides in covalent linkage.

84. The pharmaceutical composition of claim 83, wherein said chimeric peptide comprises a first β₂GPI-derived peptide having a sequence as set forth in one of SEQ ID NOs: 25-57,315 covalently linked to a second β₂GPI-derived peptide having a sequence as set forth in any of SEQ ID NOs: 25-57,315.

85. The pharmaceutical composition of claim 78, further comprising a therapeutically effective amount of at least one additional compound selected from the group consisting of HMGCoA reductase inhibitors (statins), mucosal adjuvants, corticosteroids, anti-inflammatory compounds, analgesics, growth factors, toxins, and additional tolerizing antigens.

86. An article of manufacture, packaged and identified for use in the prevention and/or treatment of a vascular condition in a subject in need thereof comprising a packaging material and a mucosal tolerance-inducing amount of an active ingredient comprising at least one beta₂-glycoprotein-1 (β₂GPI)-derived peptide, and wherein said packaging material comprises a label or package insert indicating that said mucosal tolerance-inducing amount of said active ingredient is for prevention and/or treatment of a vascular condition in the subject via mucosal administration.

87. The article of manufacture of claim 86, wherein said least one beta₂-glycoprotein-1 (β₂GPI)-derived peptide is a combination of at least two beta₂-glycoprotein-1 (β₂GPI)-derived peptides.

88. An article of manufacture, packaged and identified for use in modulating an immune response to an atheroma plaque-related antigen in a subject in need thereof comprising a packaging material and a mucosal tolerance-inducing amount of an active ingredient comprising at least one beta₂-glycoprotein-1 (β₂GPI)-derived peptide, and wherein said packaging material comprises a label or package insert indicating that said mucosal tolerance-inducing amount of said active ingredient is for modulating an immune response to an atherosclerostic plaque antigen in the subject via mucosal administration.

89. The article of manufacture of claim 88, wherein said at least one β₂GPI-derived peptide is a combination of at least two beta₂-glycoprotein-1 (β₂GPI)-derived peptides.
